(19) United States
(12) Patent Application Publication
(10) Pub. No.: US 2024/0355417 A1
(43) Pub. Date: Oct. 24, 2024

(54) SYSTEMS AND METHODS FOR DETECTION, MONITORING, AND INTERACTIVE DISPLAY OF CIRCULATING INFECTIOUS DISEASES AND THEIR CHARACTERISTICS

(71) Applicants: BioNTech SE, Mainz (DE); InstaDeep Ltd, London (GB)

(72) Inventors: Alexander Muik, Seeheim-Jugenheim (DE); Ugur Sahin, Mainz (DE); Asaf Poran, Stoneham, MA (US); Alexandra Walls, Cambridge, MA (US); Yunguan Fu, London (GB); Rafal Okuniewski, Warsaw (PL); Nicolas Winfried Pfeuffer, Frankfurt am Main (DE)

(21) Appl. No.: 18/618,412

(22) Filed: Mar. 27, 2024

Related U.S. Application Data
(60) Provisional application No. 63/551,005, filed on Feb. 7, 2024.
(60) Provisional application No. 63/591,956, filed on Oct. 20, 2023.
(60) Provisional application No. 63/492,342, filed on Mar. 27, 2023.

(51) Int. Cl. G16B 20/50; G16B 40/20; G16B 45/00; G16B 50/30; G16H 50/80

(52) U.S. Cl. CPC G16B 20/50; G16B 40/20; G16B 45/00; G16B 50/30; G16H 50/80

(57) ABSTRACT

The present disclosure, among other things, provides technologies for identifying, characterizing, and/or monitoring variant sequences of a particular reference infections agent. Among other things, systems, methods, and architectures described herein provide visualization and decision support tools that can, e.g., facilitate decision making processes by local authorities and improve pandemic response in terms of, e.g., resource allocation, policy making, and speed tailored vaccine development. The present disclosure also provides tools for analyzing circulating variants to predict mutations likely to increase immune evasion of infectious agents.

### CROSS-REFERENCE TO RELATED APPLICATIONS

This application claims priority to and benefit from U.S. Provisional 63/492,342, filed Mar. 27, 2023, U.S. Provisional Application No. 63/591,956, filed Oct. 20, 2023, and U.S. Provisional Application No. 63/551,005, filed Feb. 7, 2024, the content of each of which is incorporated by reference herein in its entirety.

### BACKGROUND OF THE INVENTION

The ongoing COVID-19 pandemic is leading to the discovery of hundreds of novel SARS-CoV-2 variants on a near daily basis. While most variants do not impact the course of the pandemic, some variants pose significantly increased risk when the acquired mutations allow better evasion of antibody neutralization in previously infected or vaccinated subjects, or increased transmissibility.

### SUMMARY

The present disclosure, among other things, provides technologies for identifying, characterizing, and/or monitoring variant sequences of a particular reference infections agent. Among other things, systems, methods, and architectures described herein provide visualization and decision support tools that can, e.g., facilitate decision making processes by local authorities and improve pandemic response in terms of, e.g., resource allocation, policy making, and speed tailored vaccine development. The present disclosure also provides tools for analyzing circulating variants to predict mutations likely to increase immune evasion of infectious agents.

Technologies described herein are relevant for, but not limited to, pathogens such as viral pathogens, such as SARS-CoV 2, influenza, norovirus, filoviruses, as well as bacteria and parasites (e.g., malaria).

In some aspects, the present disclosure provides methods for predicting one or more mutations of a viral variant likely to increase immune evasion, the methods comprising: (a) receiving and/or accessing, by a processor of a computing device, sequence data for a selected variant polypeptide (e.g., a SARS-CoV-2 spike protein variant), wherein the selected variant polypeptide is a selected variant of a particular reference polypeptide [e.g., the selected variant sequence data representing at least a portion of the selected variant polypeptide and comprising an amino acid sequence of the selected variant polypeptide portion and/or genetic (e.g., DNA, RNA) sequence that encodes the selected variant polypeptide]; (b) generating, by the processor, a plurality of candidate mutations [e.g., each candidate mutation identifying a particular (e.g., mutated) amino acid site of the selected variant polypeptide]; (c) generating, by the processor, a plurality of candidate mutation combinations, each candidate mutation combination comprising a particular combination of one or more of the plurality of candidate mutations and determining, for each candidate mutation combination, a corresponding value of a neutralization score {e.g., wherein, for a particular candidate mutation combination, the neutralization score measures a potential and/or likelihood of a mutated version of the selected variant polypeptide having the particular candidate mutation combination to evade an immune response}, thereby determining, values of the neutralization score for each of the plurality of candidate mutation combinations; (d) selecting, by the processor, based on the values of the neutralization score determined for the plurality of candidate mutation combinations, at least one of the particular candidate mutation combinations as a set of predicted mutations that are likely to increase immune evasion; and (e) storing and/or providing, by the processor, the set of predicted mutations for display and/or further processing.

In certain embodiments, sequence data for the selected variant polypeptide comprises sequences of one or more particular subunits of the viral polypeptide.

In certain embodiments, sequence data for the selected variant polypeptide comprises one or both of (i) and (ii) as follows: (i) a sequence of a receptor binding domain (RBD) of a SARS-CoV-2 spike protein variant; and (ii) a sequence of a N-terminal domain (NTD) of a SARS-CoV-2 spike protein variant.

In certain embodiments, provided methods comprise: receiving and/or accessing, by the processor, a plurality of submitted variant sequences, each submitted variant sequence representing at least a portion of a circulating variant of the reference polypeptide (e.g., having been collected and sequenced); determining, by the processor, for each of the plurality of submitted variant sequences, values one or more immune escape scores [e.g., each characterizing (e.g., quantifying) a potential for the circulating variant polypeptide to evade detection and/or neutralization by a host immune response (e.g., antibodies and/or T-Cells)]; selecting, by the processor, a subset of the submitted variant sequences based on the values of the one or more immune escape scores determined for the submitted variant sequences [e.g., the subset comprising a predetermined number (e.g., top-20) of variant sequences having a highest immune escape score value]; and performing, by the processor, for each particular submitted variant sequence of the selected subset, steps (a)-(e) using the particular submitted variant sequence as the selected variant sequence, thereby determining, for each sequence of the selected subset of submitted variant sequences, a corresponding set of predicted mutations.

In certain embodiments, one or more immune escape scores comprise an epitope alteration score and/or a semantic change score.

In certain embodiments, step (b) comprises selecting, as the plurality of candidate mutations, a subset of potential mutations having been identified in previously sequenced variant polypeptides [e.g., selecting, as the subset, those mutations having appeared at least threshold number (e.g., five) of times].

In certain embodiments, step (b) comprises: generating a plurality of potential mutations; determining, for each of the plurality of potential mutations, values of an infectivity/fitness score (e.g., a machine-learning based log-likelihood score); and selecting a subset of the plurality of potential mutations for use as the plurality of candidate mutations based on the determined infectivity/fitness score values.

In certain embodiments, a neutralization score is determined, for a particular candidate mutation combination, based on (e.g., as a function of), for each of a plurality of (e.g., known) epitopes, a number of positions mutated on the epitope relative to a particular reference polypeptide [e.g., a selected reference, such as a wild type (e.g., Wuhan) or particular variant (e.g., Alpha)].

In certain embodiments, a neutralization score is determined, for a particular candidate mutation combination, based on (e.g., as a function of), for each of a plurality of selected epitopes, a function of a number of positions mutated on the epitope [e.g., relative to a particular reference polypeptide (e.g., a selected reference, such as a wild type (e.g., Wuhan) or particular variant (e.g., Alpha))] and wherein the method comprises one or both of the following: determining and/or selecting the plurality of selected epitopes according to a particular (e.g., received as input and/or predefined, e.g., as a setting) vaccination/breakthrough condition; and determining and/or assigning an epitope weight to each of the plurality of selected epitopes according to the particular vaccination/breakthrough condition and scaling the function of the number of mutated positions on each epitope according to the corresponding epitope weight.

In certain embodiments, steps (b)-(d) comprise: at a first, initial, step: generating a first set of candidate mutations; generating a first set of mutation combinations, each having one of the first set of candidate mutations and determining values of the neutralization score for each of the first set of mutation combinations; and selecting a portion of the first set of mutation combinations based on the values of the neutralization score; and at each of one or more subsequent steps: generating a corresponding set of candidate mutations; generating a corresponding set of mutation combinations, by adding a mutation from the current set of candidate mutations to a retained mutation combination from the prior step and determining current neutralization score values for each of the current set of mutation combinations; and selecting a portion of the current set of mutation combinations to retain for further (addition of) mutations at subsequent steps or, at a final step, as the predicted set of mutations.

In certain embodiments, provided methods comprise using an iterative and/or step-wise search technique to generate and select mutation combinations for inclusion in the set of predicted mutations (e.g., a beam search).

In certain embodiments, provided methods comprise performing steps (a)-(d) for each of a plurality of selected variant sequences (e.g., sequences of circulating variants having been identified as variants of concern) to generate a plurality of sets of predicted mutations, [e.g., one or more set(s) of predicted mutations for each of the plurality of selected variant sequences], and causing display of (e.g., via a graphical user interface (GUI) of a decision support system) the plurality of sets of predicted mutations (e.g., in a tabular format, showing, for each variant lineage, its corresponding set of predicted mutations).

In certain embodiments, provided methods comprise performing steps (a)-(d) for each of a plurality of variant sequences, each corresponding to a particular subunit or region (e.g., RBD and/or NTD) of a viral variant polypeptide.

In certain embodiments, provided methods comprise repeatedly performing steps (a)-(d) over time [e.g., at regular intervals (e.g., daily, e.g., weekly, e.g., monthly, e.g., quarterly)], thereby regularly updating the set of predicted mutations as new sequence and/or epitope data is obtained (e.g., repeatedly updating epitope data used in computing neutralization scores; e.g., repeatedly updating sequence data used for generation of candidate mutations).

In certain embodiments, provided methods comprise: repeatedly receiving and/or accessing, by the processor, (e.g., over time), sequence data comprising sequences of (e.g., newly) sequenced variants; comparing, by the processor, the sequence data to the set of predicted mutations; and responsive to identifying presence of at least a portion of the set of predicted mutations within one or more of the sequences, causing, by the processor, transmission and/or display of an alert to one or more users.

In some aspects, the present disclosure provides methods for evaluating and tracking evolution of viral lineages via an interactive decision support system, the provided methods comprising: (a) receiving and/or accessing, by a processor of a computing device, viral lineage data from one or more databases, the viral lineage data comprising, for each particular lineage of a plurality of viral lineages, one or more of (i) through (iv) as follows: (i) a lineage name identifying the particular lineage; (ii) a lineage description; (iii) a corresponding set of consensus mutations [e.g., wherein the set of consensus mutations for a particular lineage comprises mutations having been observed at least a threshold percentage (e.g., 50% or more, e.g., 60% or more, e.g., 75% or more, e.g., 80% or more, etc.) of submitted sequences for the particular lineage] for the particular lineage; and (iv) a submissions dataset comprising a plurality of submitted sequences identified as belonging to the particular lineage {e.g., and, for each submitted sequence belonging to the particular lineage, geographic identification data identifying a location where the submitted sequence was collected and/or time data identifying a date of collection and/or submission of the submitted sequence}; (b) performing, by the processor, one or both of (A) and (B) as follows: (A) causing rendering of a visual (e.g., graphical and/or textual) representation of at least a portion of the viral lineage data; and (B) using the viral lineage data to determine, for each of at least a portion of the plurality of viral lineages, values one or more epitope conservation metrics {e.g., each epitope conservation metric providing, for a particular viral lineage and particular epitope type [e.g., a B-cell neutralizing epitope; e.g., a MHC I (e.g., HLA class I) T-cell epitope; e.g., a MHC II (e.g., HLA class II) T-cell epitope; e.g., one or more specific antibody epitopes/epitope classes] a measure of (e.g., a number of) epitopes that are altered and/or unaltered (e.g., conserved) within the particular viral lineage relative to a reference lineage} and causing rendering of a graphical representation of the determined values of the one or more epitope conservation metrics for graphical display and/or providing the determined values one or more epitope conservation for further processing.

In certain embodiments, step (b) comprises (A) causing rendering of a visual representation of at least a portion of the viral lineage data, the visual representation comprising a graphical representation of a frequency (of observed sequences having a particular) of the consensus mutations of a particular focus lineage of the viral lineages (e.g., as shown in FIGS. 8K-8M).

In certain embodiments, step (b) comprises using the viral lineage data to determine the values of the one or more epitope conservation metrics and causing rendering of one or more graphical representations of the values of the epitope conservation metrics (e.g., one or more line-plots, radar plots, tabular displays, e.g., as shown in FIGS. 8B-8I).

In certain embodiments, one or more epitope alteration metrics comprise a level (e.g., a number, percentage, fraction, etc.) of one or more of the following: unaltered (e.g., conserved) B-cell naturalizing epitopes; unaltered (e.g., conserved) HLA class I T-cell epitopes; and unaltered (e.g., conserved) HLA class II T-cell epitopes.

In some aspects, the present disclosure provides methods for forecasting prevalence and/or distribution of a plurality variants of a circulating pathogen (e.g., virus), the method comprising: (a) receiving and/or accessing, by a processor of a computing device, sequence data (e.g., historical sequence data) for the circulating pathogen, the sequence data comprising, a plurality of variant sequences, each (i) representing a (e.g., nucleic acid; e.g., amino acid) sequence of a variant of a particular polypeptide (e.g., a particular protein and/or portion thereof) of the circulating pathogen and (ii) associated with a particular time (e.g., when the sequence of the variant was determined and/or submitted to a database); (b) for each particular time point of a plurality of time points, identifying and assigning one or more of the plurality of variant sequences that are associated with the particular time point to a particular cluster of a set of (e.g., one or more; e.g., a plurality of) clusters [e.g., each cluster comprising a plurality of related (e.g., having been determined as related/similar using one or more clustering algorithms) variant sequences], thereby sub-dividing the plurality of variant sequences across the set of clusters and tracking the distribution of variant sequences across the set of clusters over time; and (c) performing, by the processor, one or both of (A) and (B) as follows: (A) causing rendering of a visual (e.g., graphical and/or textual) representation of the distribution of variant sequences across the set of clusters (e.g., at one or more of the plurality of time points); and (B) using distribution of variant sequences across the set of clusters and its variation over time to generate a projected distribution of variant sequences across the one or more clusters at a current and/or future time point.

In certain embodiments, variant sequences are sequences of variants of a particular viral polypeptide {e.g., a SARS-CoV-2 protein poly peptide and/or portion thereof [e.g., a SARS-CoV-2 spike protein and/or portion thereof (e.g., a receptor binding domain (RBD) of a SARS-CoV-2 spike protein; e.g., an N-terminal domain of a SARS-CoV-2 spike protein)]; e.g., an influenza protein polypeptide and/or portion thereof; e.g., a human immunodeficiency virus (HIV) protein and/or portion thereof}.

In certain embodiments, step (b) comprises: determining, for each of the plurality of variant sequences, using a machine learning model, a corresponding characteristic vector [e.g., wherein, for a particular variant sequence, its corresponding characteristic vector determined based on (e.g., as an average across amino acid sequence positions) an internal representation (e.g., an embedding) generated by the machine learning model based on (e.g., following receipt, as input, of) the particular variant sequence], thereby determining a plurality of characteristic vectors, each corresponding to (e.g., and representing) a particular one of the plurality of variant sequences; and using the plurality of characteristic vectors to assign each of the variant sequences to a particular cluster of the set of clusters.

In certain embodiments, provided methods comprise assigning each of the plurality of variant sequences to a particular cluster of the set of clusters based at least in part on its corresponding characteristic vector and/or one or more reduced dimensionality versions thereof.

In certain embodiments, provided methods comprise: determining, for each of the plurality of characteristic vectors, a reduced dimensionality version thereof [e.g., via a dimensionality reduction model (e.g., t-SNE, UMAP, etc.)], thereby determining, for each of the plurality of variant sequences, a corresponding reduced dimensionality characteristic vector; and assigning each of the plurality of variant sequences to a particular cluster of the set of clusters based at least in part on its corresponding reduced dimensionality characteristic vector.

In certain embodiments, a machine learning model is a large language model (LLM).

In certain embodiments, an LLM is a trained model, having been previously trained (e.g., in an unsupervised fashion) to predict, based on receipt of a partially masked and/or incomplete input polypeptide sequence, types of masked and/or remaining (e.g., next) sequence locations.

In certain embodiments, step (b) comprises: determining, by the processor, for one or more (e.g., selected) reference time point(s) [e.g., one or more initial time point(s) (e.g., historical time points, up to a particular date); e.g., a most recent time point], the set of clusters based on one or more of the plurality of variant sequences associated with the one or more reference time point(s) [e.g., using one or more clustering algorithms (e.g., k-means clustering) to identify one or more clusters of variant sequences within the variant sequences associated with the reference time point(s) (e.g., based on embedding vectors of the variant sequences associated with the reference time point(s))]; and at other time points, assigning, by the processor, variant sequences (associated with the other time points) to a particular cluster of the set of clusters determined at for the reference time point(s).

In certain embodiments, provided methods comprise: determining, by the processor, a reference set of variant sequences comprising a plurality of variant sequences associated with each of one or more reference time point(s); using the reference set of variant sequences to generate a training dataset comprising characteristic vectors and/or reduced dimensionality versions thereof for each of the variant sequences of the reference set; and training a clustering model according to the training dataset (e.g., using the training dataset to train a clustering model), thereby determining a trained clustering model and the set of clusters (e.g., wherein the trained clustering model receives, as input, a characteristic vector corresponding to a particular variant sequence, and/or reduced dimensionality version thereof, and determines, as output an assignment of a particular cluster of the set of clusters for the particular variant sequence).

In certain embodiments, provided methods comprise using the trained clustering model to assign variant sequences to a particular cluster of the set of clusters.

In certain embodiments, provided methods comprise using the training dataset to train a dimensionality reduction model (e.g., a t-SNE model, a UMAP model, etc.) to determine the reduced dimensionality versions of characteristic vectors corresponding to the plurality of variant sequences.

In certain embodiments, step (b) comprises: determining, by the processor, at each particular time point of the plurality of time points, an initial set of clusters based on one or more of the plurality of variant sequences associated with the particular time point [e.g., using one or more clustering algorithms (e.g., k-means clustering) to identify one or more clusters of variant sequences within the variant sequences associated with the reference time point (e.g., based on characteristic vectors of the variant sequences associated with the reference time point)], thereby determining a plurality of initial sets of clusters, each associated with a particular time point of the plurality of time points; and matching, by the processor, corresponding clusters between the plurality of initial sets of clusters [e.g., based on one or more computed metrics, such as centers of mass (e.g., and/or distances therebetween), size, etc.] to determine, and assign variant sequences to, a common set of clusters.

In certain embodiments, provided methods comprise determining values of one or more cluster performance scores measuring a clustering performance (e.g., consistency scores, such as a silhouette score, Calinski-Harabasz Index, Davies-Bouldin index, etc.).

In certain embodiments, provided methods comprise: determining, by the processor, based on the one or more cluster performance scores, a reduction in the clustering performance [e.g., by determining one or more values (e.g., and/or functions thereof) of the one or more cluster performance scores to fall below a particular threshold value; e.g., by determining a decrease (e.g., in comparison with one or more selected time points) in value(s) of one or more of the cluster performance scores] [e.g., upon receipt of new variant sequence submissions]; and causing, by the processor, rendering [e.g., within a decision support graphical user interface (GUI)] of a graphical indicator [e.g., appearance of an on-screen icon, visual flag (e.g., a change in color, size, font, animation of, etc., to an existing graphical icon), pop-up message, etc.] alerting a user to the reduction in the clustering performance.

In certain embodiments, provided methods comprise: determining, by the processor, based on the one or more cluster performance scores, a reduction in the clustering performance [e.g., by determining one or more values (e.g., and/or functions thereof) of the one or more cluster performance scores to fall below a particular threshold value; e.g., by determining a decrease (e.g., in comparison with one or more selected time points) in value(s) of one or more of the cluster performance scores] [e.g., upon receipt of new variant sequence submissions]; and based on the determined reduction in the clustering performance, (e.g., automatically) updating (e.g., re-training) one or both of (i) a dimensionality reduction model and (ii) a clustering model.

In some aspects, the present disclosure provides methods for providing a decision support interface for identifying and/or assessing risk of one or more variant polypeptides, the method comprising causing, by a processor of a computing device, rendering of a decision support graphical user interface (GUI) for display on a local user device (e.g., a local health authority, government system, etc.), the decision support GUI comprising one or more data display pages, each comprising one or more graphical charts comprising graphical renderings (e.g., graphs, tables, heatmaps, etc.) (e.g., that visually convey and contextualize) of viral lineage, infectivity, immune escape, and/or overall risk data for the one or more variant polypeptides [e.g., a particular virus (e.g., SARS-CoV-2) and (e.g., circulating) variants thereof].

In certain embodiments, one or more display pages comprise a dashboard, showing a total spike count graph and/or a geographic lineage distribution (e.g., as shown in FIGS. 4A and 4B).

In certain embodiments, one or more display pages comprise a color-coded heatmap chart displaying a list of potential mutations and, for each of a plurality of variant lineages (e.g., a top 20 immune escaping variants) an indication (e.g., color-coded) of a frequency of each of the potential mutations (e.g., as shown in FIGS. 5A and 5B).

In certain embodiments, one or more display pages comprise a tabular display showing a plurality of lineages and, for each, predicted immune escaping mutations generated via a forward-looking system (e.g., as shown in FIG. 6).

In certain embodiments, one or more display pages comprise a sequence comparison page comprising, for each of two or more selected variants, graphical renderings comparing (i) a frequency of potential mutations in the two or more variants and/or (ii) sequences of the two or more variants and/or (iii) three dimensional structures of the two or more variants (e.g., as shown in FIGS. 7A-C).

In certain embodiments, one or more display pages comprise a lineage description page displaying a plurality of lineages and descriptions thereof, along with, for each linage, a list of its consensus mutations (e.g., and a search box for user entry and selection of a particular focus lineage for further review and/or analysis) (e.g., as shown in FIG. 8A).

In certain embodiments, one or more display pages comprise one or more lineage focus pages, each displaying one or more of the following: a sequence heatmap comprising, for a particular selected focus lineage, a graphical rendering conveying a frequency and/or number of observed sequences having each of one or more particular mutations [e.g., a plurality of mutations being monitored or observed (e.g., consensus mutations for the particular selected focus lineage)] (e.g., as shown in FIG. 8B); one or more graphical charts and/or tables displaying, for each of a plurality of lineages, including the selected focus lineage (e.g., and a wild-type and/or reference lineage) values of one or more epitope conservation metrics (e.g., line plots, tables, radar plots, etc.; e.g., as shown in FIGS. 8B-8I); and one or more graphical charts and/or tables displaying, for one or more particular selected lineages (e.g., the selected focus lineage), a change in overall number of submitted sequences over time, and/or a change in number of distinct geographic locations (e.g., cities, counties, countries, etc.) in which the one or more particular selected lineages originated (e.g., were collected from or otherwise identified as originating from) over time (e.g., as shown in FIGS. 8J-8N).

In certain embodiments, one or more display pages comprise one or more cluster analytics pages, each comprising graphical rendering (e.g., a pie-chart) visually representing a grouping of plurality of viral variant lineages into a plurality of clusters, each of the plurality of clusters comprising a particular (e.g., distinct) subset of the plurality of viral variant lineages.

In some aspects, the present disclosure provides systems for predicting one or more mutations of a viral variant likely to increase immune evasion, the systems comprising: a processor of a computing device; and memory having instructions stored thereon, wherein the instructions, when executed by the processor, cause the processor to: (a) receive and/or access sequence data for a selected variant polypeptide (e.g., a SARS-CoV-2 spike protein variant), wherein the selected variant polypeptide is a selected variant of a particular reference polypeptide [e.g., the selected variant sequence data representing at least a portion of the selected variant polypeptide and comprising an amino acid sequence of the selected variant polypeptide portion and/or genetic (e.g., DNA, RNA) sequence that encodes the selected variant polypeptide]; (b) generate a plurality of candidate mutations [e.g., each candidate mutation identifying a particular (e.g., mutated) amino acid site of the selected variant polypeptide]; (c) generate a plurality of candidate mutation combinations, each candidate mutation combination comprising a particular combination of one or more of the plurality of candidate mutations and determining, for each candidate mutation combination, a corresponding value of a neutralization score {e.g., wherein, for a particular candidate mutation combination, the neutralization score measures a potential and/or likelihood of a mutated version of the selected variant polypeptide having the particular candidate mutation combination to evade an immune response}, thereby determining, values of the neutralization score for each of the plurality of candidate mutation combinations; (d) select, based on the values of the neutralization score determined for the plurality of candidate mutation combinations, at least one of the particular candidate mutation combinations as a set of predicted mutations that are likely to increase immune evasion; and (e) store and/or provide the set of predicted mutations for display and/or further processing.

In some aspects, the present disclosure provides systems for evaluating and tracking evolution of viral lineages via an interactive decision support system, the systems comprising: a processor of a computing device; and memory having instructions stored thereon, wherein the instructions, when executed by the processor, cause the processor to: (a) receive and/or access viral lineage data from one or more databases, the viral lineage data comprising, for each particular lineage of a plurality of viral lineages, one or more of (i) through (iv) as follows: (i) a lineage name identifying the particular lineage; (ii) a lineage description; (iii) a corresponding set of consensus mutations [e.g., wherein the set of consensus mutations for a particular lineage comprises mutations having been observed at least a threshold percentage (e.g., 50% or more, e.g., 60% or more, e.g., 75% or more, e.g., 80% or more, etc.) of submitted sequences for the particular lineage] for the particular lineage; and (iv) a submissions dataset comprising a plurality of submitted sequences identified as belonging to the particular lineage {e.g., and, for each submitted sequence belonging to the particular lineage, geographic identification data identifying a location where the submitted sequence was collected and/or time data identifying a date of collection and/or submission of the submitted sequence}; (b) perform one or both of (A) and (B) as follows: (A) cause rendering of a visual (e.g., graphical and/or textual) representation of at least a portion of the viral lineage data; and (B) use the viral lineage data to determine, for each of at least a portion of the plurality of viral lineages, values one or more epitope conservation metrics {e.g., each epitope conservation metric providing, for a particular viral lineage and particular epitope type [e.g., a B-cell neutralizing epitope; e.g., a MHC I (e.g., HLA class I) T-cell epitope; e.g., a MHC II (e.g., HLA class II) T-cell epitope; e.g., one or more specific antibody epitopes/epitope classes] a measure of (e.g., a number of) epitopes that are altered and/or unaltered (e.g., conserved) within the particular viral lineage relative to a reference lineage} and cause rendering of a graphical representation of the determined values of the one or more epitope conservation metrics for graphical display and/or provide the determined values one or more epitope conservation for further processing.

In some aspects, the present disclosure provides systems for forecasting prevalence and/or distribution of a plurality variants of a circulating pathogen (e.g., virus), the systems comprising: a processor of a computing device; and memory having instructions stored thereon, wherein the instructions, when executed by the processor, cause the processor to: (a) receive and/or access sequence data (e.g., historical sequence data) for the circulating pathogen, the sequence data comprising, a plurality of variant sequences, each (i) representing a (e.g., nucleic acid; e.g., amino acid) sequence of a variant of a particular polypeptide (e.g., a particular protein and/or portion thereof) of the circulating pathogen and (ii) associated with a particular time (e.g., when the sequence of the variant was determined and/or submitted to a database); (b) for each particular time point of a plurality of time points, identify and assign one or more of the plurality of variant sequences that are associated with the particular time point to a particular cluster of a set of (e.g., one or more; e.g., a plurality of) clusters [e.g., each cluster comprising a plurality of related (e.g., having been determined as related/similar using one or more clustering algorithms) variant sequences], thereby sub-dividing the plurality of variant sequences across the set of clusters and tracking the distribution of variant sequences across the set of clusters over time; and (c) perform one or both of (A) and (B) as follows: (A) cause rendering of a visual (e.g., graphical and/or textual) representation of the distribution of variant sequences across the set of clusters (e.g., at one or more of the plurality of time points); and (B) use distribution of variant sequences across the set of clusters and its variation over time to generate a projected distribution of variant sequences across the one or more clusters at a current and/or future time point.

In some aspects, the present disclosure provides systems for providing a decision support interface for identifying and/or assessing risk of one or more variant polypeptides, the systems comprising: a processor of a computing device; and memory having instructions stored thereon, wherein the instructions, when executed by the processor, cause the processor to: cause rendering of a decision support graphical user interface (GUI) for display on a local user device (e.g., a local health authority, government system, etc.), the decision support GUI comprising one or more data display pages, each comprising one or more graphical charts comprising graphical renderings (e.g., graphs, tables, heatmaps, etc.) (e.g., that visually convey and contextualize) of viral lineage, infectivity, immune escape, and/or overall risk data for the one or more variant polypeptides [e.g., a particular virus (e.g., SARS-CoV-2) and (e.g., circulating) variants thereof].

Features of embodiments described with respect to one aspect may be applied with respect to other aspects.

### BRIEF DESCRIPTION OF THE DRAWING

The foregoing and other objects, aspects, features, and advantages of the present disclosure will become more apparent and better understood by referring to the following description taken in conjunction with the accompanying drawings, in which:

FIG. 1 is a schematic illustrating an example process for determining and combining viral variant risk scores, according to an illustrative embodiment.

FIG. 2 is a block flow diagram of an example process for using a machine learning (e.g., language) model to determine immune escape (semantic change) and fitness (log-likelihood) scores, according to an illustrative embodiment.

FIG. 3A is a block flow diagram showing an example process for predicting mutations likely to increase immune evasion, according to an illustrative embodiment, according to an illustrative embodiment.

FIG. 3B is a block flow diagram illustrating an example step in a process for predicting mutations likely to increase immune evasion, according to an illustrative embodiment.

FIG. 4A is a screenshot of an example graphical user interface (GUI) dashboard for monitoring evolution of viral lineages, according to an illustrative embodiment.

FIG. 4B is an expanded view of two graphical data displays shown in the screenshot of FIG. 4A, according to an illustrative embodiment.

FIG. 5A is a screenshot of an example GUI for displaying risk data for set of viral variants, accordingly to an illustrative embodiment.

FIG. 5B is an expanded view of a bitmap/heatmap data display shown in the screenshot of FIG. 5A that conveys frequencies of various mutations in viral variants, according to an illustrative embodiment.

FIG. 5C is an expanded view of a graph showing sequence growth, included in the screenshot shown in FIG. 5A, according to an illustrative embodiment.

FIG. 5D is an expanded view of a scatter plot shown in the screenshot of FIG. 5A, which graphs percentages of unaltered T-cell and B-cell epitopes for various viral variants, according to an illustrative embodiment.

FIG. 5E is an expanded view of a radar plot shown in the screenshot of FIG. 5A, which graphs percentages of unaltered HLA class I T-cell epitopes, unaltered HLA class II T-cell epitopes, and unaltered neutralizing B-cell epitopes for various viral variants, according to an illustrative embodiment.

FIG. 6 is a screenshot of a GUI for displaying predicted mutations for a set of variant lineages, according to an illustrative embodiment.

FIG. 7A is a screenshot of a GUI for comparing sequences of two viral variants, according to an illustrative embodiment.

FIG. 7B is an expanded view of a bitmap/heatmap data display shown in the screenshot of FIG. 7A that conveys frequencies of various mutations in two viral variants, being compared, according to an illustrative embodiment.

FIG. 7C is an expanded view of a portion of the screenshot shown in FIG. 7A, showing graphical renderings of sequences and structures of two variants being compared, according to an illustrative embodiment.

FIG. 8A is a screenshot of a GUI for allowing a user to view, select, and/or search for particular lineages of interest, according to an illustrative embodiment.

FIG. 8B is a screenshot of a portion of a GUI for allowing a user to view and analyze mutations present in a selected focus linage and compare their impact on various epitopes with those of other mutations, according to an illustrative embodiment.

FIG. 8C is an expanded view of a portion of the screenshot shown in FIG. 8B showing text and a bitmap/heatmap rendered and conveying mutations in a particular viral variant lineage, according to an illustrative embodiment.

FIG. 8D is an expanded view of a portion of the screenshot shown in FIG. 8B showing two rendered graphs plotting relative fractions of unaltered HLA class I and II T-cell epitopes and B-cell epitopes, according to an illustrative embodiment.

FIG. 8E is a screenshot of another portion of the GUI for allowing a user to view and analyze mutations present in a selected focus linage and compare their impact on various epitopes with those of other mutations, according to an illustrative embodiment.

FIG. 8F is an expanded view of a portion of the screenshot shown in FIG. 8E, showing a graphical rendering of a table showing conservation of B-cell and T-cell immunity, according to an illustrative embodiment.

FIG. 8G is a screenshot of another portion of the GUI for allowing a user to view and analyze mutations present in a selected focus linage and compare their impact on various epitopes with those of other mutations, according to an illustrative embodiment.

FIG. 8H is an expanded view of a portion of the screenshot shown in FIG. 8G, showing three graphs plotting relative fractions of conserved epitopes of various types, including HLA class I T-cell epitopes, HLA class II T-cell epitopes, epitopes associated with various regions of an antigen (e.g., NTD epitopes, RBD epitopes, and other epitopes), and various RBD classes of epitopes, according to an illustrative embodiment.

FIG. 8I is an expanded view of a portion of the screenshot shown in FIG. 8G, showing a graphical rendering of a table conveying conservation of various types of B-cell epitopes, according to an illustrative embodiment.

FIG. 8J is a screenshot of a GUI displaying submission statistics for a selected focus lineage, according to an illustrative embodiment.

FIG. 8K is a screenshot of a GUI displaying submission statistics, according to an illustrative embodiment.

FIG. 8L is a screenshot of a GUI displaying comparing submitted sequences and/or mutations thereof for a selected focus lineage, according to an illustrative embodiment.

FIG. 8M is an expanded view of a portion of the screenshot shown in FIG. 8L, showing a bitmap/heatmap rendered and conveying mutations in certain viral variant lineages, according to an illustrative embodiment.

FIG. 8N is an expanded view of a portion of the screenshot shown in FIG. 8L, showing a graphical rendering of a table providing a listing of submitted sequences, according to an illustrative embodiment.

FIG. 9A is a screenshot of a portion of another GUI for allowing a user to view and analyze mutations present in a selected focus linage and compare their impact on various epitopes with those of other mutations, according to an illustrative embodiment.

FIG. 9B is a screenshot of a portion of a GUI allowing a user to view geographic distribution of lineage submissions, according to an illustrative embodiment.

FIG. 9C is a screenshot of a GUI displaying comparing submitted sequences and/or mutations thereof for a selected focus lineage, according to an illustrative embodiment.

FIG. 9D is a screenshot of a portion of a GUI showing a graphical rendering of a table providing a listing of submitted sequences, according to an illustrative embodiment.

FIG. 9E is a screenshot of a portion of a GUI showing two rendered graphs plotting relative fractions of unaltered HLA class I and II T-cell epitopes and B-cell epitopes, according to an illustrative embodiment.

FIG. 9F is a screenshot of a portion of a GUI showing a graphical rendering of a table conveying conservation of various types of B-cell epitopes, according to an illustrative embodiment.

FIG. 9G is a screenshot of a portion of a GUI showing three graphs plotting relative fractions of conserved epitopes of various types, including HLA class I T-cell epitopes, HLA class II T-cell epitopes, epitopes associated with various regions of an antigen (e.g., NTD epitopes, RBD epitopes, and other epitopes), and various RBD classes of epitopes, according to an illustrative embodiment.

FIG. 10 is a screenshot of a GUI providing weekly downloadable reports, according to an illustrative embodiment.

FIG. 11A is a screenshot of portions of a GUI displaying documentation describing data sources and data analysis processes used by various technologies described herein, according to various illustrative embodiments.

FIG. 11B is a screenshot of portions of a GUI displaying documentation describing data sources and data analysis processes used by various technologies described herein, according to various illustrative embodiments.

FIG. 11C is a screenshot of portions of a GUI displaying documentation describing data sources and data analysis processes used by various technologies described herein, according to various illustrative embodiments.

FIG. 11D is a screenshot of portions of a GUI displaying documentation describing data sources and data analysis processes used by various technologies described herein, according to various illustrative embodiments.

FIG. 11E is a screenshot of portions of a GUI displaying documentation describing data sources and data analysis processes used by various technologies described herein, according to various illustrative embodiments.

FIG. 11F is a screenshot of portions of a GUI displaying documentation describing data sources and data analysis processes used by various technologies described herein, according to various illustrative embodiments.

FIG. 12 is a screenshot of a GUI allowing a user to search for references pertaining to a particular viral variant, according to an illustrative embodiment.

FIG. 13A is a block flow diagram of an example process for identifying clusters of variant sequences and rendering them for display and/or using them to generate forecasts, according to an illustrative embodiment.

FIG. 13B is an illustrative (e.g., hypothetical) schematic of a plurality of characteristic vectors generated for variant sequences and identified clusters thereof, according to an illustrative embodiment.

FIG. 13C is an illustrative (e.g., hypothetical) graph showing tracking and forecasting cluster sizes over time, according to an illustrative embodiment.

FIG. 14A is a screenshot of portions of an example GUI allowing users to view and track clusters (and/or related lineages) of viral variant sequences over time, according to various illustrative embodiments.

FIG. 14B is a screenshot of portions of an example GUI allowing users to view and track clusters (and/or related lineages) of viral variant sequences over time, according to various illustrative embodiments.

FIG. 14C is a screenshot of portions of an example GUI allowing users to view and track clusters (and/or related lineages) of viral variant sequences over time, according to various illustrative embodiments.

FIG. 14D is a screenshot of portions of an example GUI allowing users to view and track clusters (and/or related lineages) of viral variant sequences over time, according to various illustrative embodiments.

FIG. 14E is a screenshot of portions of an example GUI allowing users to view and track clusters (and/or related lineages) of viral variant sequences over time, according to various illustrative embodiments.

FIG. 15A is a screenshot of portions of an example GUI allowing users to view cluster analytics and their relation to viral variant lineages, according to various illustrative embodiments.

FIG. 15B is an expanded view of a portion of the screenshot shown in FIG. 15A, showing graphical rendering of a cluster centric view of associations between lineages and clusters, according to an illustrative embodiment.

FIG. 15C is an expanded view of another portion of the screenshot shown in FIG. 15A, showing a bitmap/heatmap graphical rendering for comparison of mutations in two viral variants, according to an illustrative embodiment.

FIG. 15D is a screenshot of portions of an example GUI allowing users to view cluster analytics and their relation to viral variant lineages, according to various illustrative embodiments.

FIG. 15E is an expanded view of a portion of the screenshot shown in FIG. 15D, showing graphical rendering of a cluster centric view of associations between lineages and clusters, according to an illustrative embodiment.

FIG. 15F is an expanded view of another portion of the screenshot shown in FIG. 15D, showing a bitmap/heatmap graphical rendering for comparison of mutations in two viral variants, according to an illustrative embodiment.

FIG. 15G is a screenshot of portions of an example GUI allowing users to view cluster analytics and their relation to viral variant lineages, according to various illustrative embodiments.

FIG. 15H is an expanded view of a portion of the screenshot shown in FIG. 15G, showing graphical rendering of a lineage centric view of associations between lineages and clusters, according to an illustrative embodiment.

FIG. 15I is an expanded view of another portion of the screenshot shown in FIG. 15G, showing a bitmap/heatmap graphical rendering for comparison of mutations in two viral variants, according to an illustrative embodiment.

FIG. 15J is a screenshot of portions of an example GUI allowing users to view cluster analytics and their relation to viral variant lineages, according to various illustrative embodiments.

FIG. 15K is an expanded view of a portion of the screenshot shown in FIG. 15J, showing graphical rendering of a lineage centric view of associations between lineages and clusters, according to an illustrative embodiment.

FIG. 15L is an expanded view of another portion of the screenshot shown in FIG. 15J, showing a bitmap/heatmap graphical rendering for comparison of mutations in two viral variants, according to an illustrative embodiment.

FIG. 15M is a screenshot of portions of an example GUI allowing users to view cluster analytics and their relation to viral variant lineages, according to various illustrative embodiments.

FIG. 15N is a screenshot of portions of an example GUI allowing users to view cluster analytics and their relation to viral variant lineages, according to various illustrative embodiments.

FIG. 16 is a block diagram of an exemplary cloud computing environment, used in certain embodiments.

FIG. 17 is a block diagram of an example computing device and an example mobile computing device used in certain embodiments.

FIG. 18 is a set of graphs showing optimization of hyperparameters in an example forward looking system.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### Certain Definitions

About or Approximately: The term “about” or “approximately”, when used herein in reference to a value, refers to a value that is similar to the referenced value. In general, those skilled in the art, familiar with the context, will appreciate the relevant degree of variance encompassed by “about” or “approximately” in that context. For example, in some embodiments, the term “about” or “approximately” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Agent: In general, the term “agent”, as used herein, is used to refer to an entity (e.g., for example, a lipid, metal, nucleic acid, polypeptide, polysaccharide, small molecule, etc., or complex, combination, mixture or system [e.g., cell, tissue, organism] thereof), or phenomenon (e.g., heat, electric current or field, magnetic force or field, etc.). In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Amino acid: in its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated CDRs or sets thereof, single chain Fvs; polypeptide-Fc fusions; single domain antibodies, alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, single domain antibodies, trifunctional antibodies, diabodies, and minibodies. etc. In some embodiments, relevant formats may be or include: Adnectins®; Affibodies®; Affilins®; Anticalins®; Avimers®; BiTE®s; cameloid antibodies; Centyrins®; ankyrin repeat proteins or DARPINs®; dual-affinity re-targeting (DART) agents; Fynomers®; shark single domain antibodies such as IgNAR; immune mobilixing monoclonal T cell receptors against cancer (ImmTACs); KALBITOR®s; MicroProteins; Nanobodies® minibodies; masked antibodies (e.g., Probodies®); Small Modular ImmunoPharmaceuticals (“SMIPs™”); single chain or Tandem diabodies (TandAb®); TCR-like antibodies; Trans-bodies®; TrimerX®; VHHs. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Antigen: The term “antigen”, as used herein, refers to an agent that elicits an immune response; and/or an agent that binds to a T cell receptor (e.g., when presented by an MHC molecule) or to an antibody. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, and antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer), etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present disclosure are provided in a crude form. In some embodiments, an antigen is a recombinant antigen.

Antigen presenting cell: The phrase “antigen presenting cell” or “APC,” as used herein, has its art understood meaning referring to cells which process and present antigens to T-cells. Exemplary antigen cells include dendritic cells, macrophages and certain activated epithelial cells.

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Epitope: as used herein, the term “epitope” refers to a moiety that is specifically recognized, or predicted to be recognized, by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Epitope Alteration Score: As used interchangeably herein, the terms “epitope alteration score” and “epitope score” both refer to a measure of alteration to a viral polypeptide at epitope positions. In some embodiments, such alteration can be characterized by the impact of mutation(s) in one or more epitopes of a viral variant on recognition by antibodies (e.g., neutralizing antibodies). For example, in some embodiments, such alteration can be characterized by determining the number of antibodies potentially escaped. In some embodiments, antibodies for characterization have been isolated from patients who have been vaccinated against a disease or who have previously been infected with a disease (e.g., SARS-CoV-2). In some embodiments, antibodies for characterization have previously been shown to bind a reference sequence. In some embodiments, an epitope alteration score can be determined by comparison of mutations in a variant candidate to one or more regions of a reference sequence that have previously been shown to bind antibodies (e.g., through structural data). In some embodiments, an epitope alteration score can be determined by enumerating the number of unique epitopes involving altered positions, as measured across one or more known antibody-viral polypeptide complex structures (e.g., all known antibody-viral polypeptide complex structures).

In some embodiments, an epitope alteration score is a measure of how many distinct epitopes are evaded by a variant candidate as compared to a reference sequence (e.g., as compared to a wild type sequence). In some embodiments, an epitope alteration score is computed based on known binding sites of antibodies, e.g., as reported in Protein Data Bank. In some embodiments, an epitope alteration score can change over time with identification of new epitope positions and/or discoveries of epitope-binding antibodies. In some embodiments, an epitope alteration score can be used to characterize degree of alteration of a SARS-CoV-2 Spike polypeptide at epitope positions, for example, in some embodiments by counting the number or percentage of antibodies potentially escaped. In various embodiments described herein, an epitope alteration score can be normalized such that it ranks between 0 and 100%.

Growth score: As used interchangeably herein, the term “growth,” “growth metric,” or “growth score” refers to a measure of the rate at which a given variant is growing in a subject population (e.g., at a given time). In some embodiments, a growth score refers to lineage-level growth. For example, in some embodiments, a growth score of a given variant can be determined by referencing growth of a parent species or a known variant of substantially the same lineage, or a known variant having a similar sequence (e.g., a sequence that is at least 90% identical to the given variant). In some embodiments, growth of a given variant is a function of the change in the number of subjects within a subject population who are reported as being infected with the given variant over a given time period relative to a reference infection rate (e.g., a reference infection rate determined over a defined period of time). In some embodiments, growth of a given variant is a function of the change in the proportion of a subject population infected with the given variant over a given time period relative to a reference infection rate (e.g., a reference infection rate determined over a defined period of time). In some embodiments, a growth score of a given variant can be an empirically determined by considering sequences associated with a given variant (e.g., in some embodiments including sequences associated with a lineage) that have been observed within a defined period and computing its proportion among all observed sequences at a given time relative to a reference level (e.g., its proportion determined over a defined period of time). For example, in some embodiments, for each lineage, its proportion of sequences among all observed sequences is calculated for an extended period of time (e.g., an eight-week window) and for the most recent time window (e.g., for the last 24 hours, last 48 hours, last 72 hours, last 4 days, last 5 days, last 6 days, or last week), denoted by r_{extended} and rₗₐₛₜ, respectively. The growth of the lineage is defined by their ratio r_{extended}/rₗₐₛₜ, measuring the change of the proportion. In various embodiments described herein, a growth score can be normalized such that it ranks between 0 and 100%.

Human: In some embodiments, a human is an embryo, a fetus, an infant, a child, a teenager, an adult, or a senior citizen.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Immune Escape Score”: As used herein, the term “immune escape score” refers to a measure of a viral variant's ability to escape detection and/or neutralization by antibodies (e.g., neutralization antibodies generated by a patient that has previously been infected and/or vaccinated against a reference sequence). In some embodiments, determination of an immune escape score comprises calculating a semantic change score (e.g., a semantic change score determined using a method disclosed herein). In some embodiments, determination of an immune escape score comprises calculation of an epitope alteration score (e.g., using one of the methods described herein). In some embodiments, the immune escape score is determined using a combination of an epitope alteration score and a semantic change score. In some embodiments, the immune escape score is an average of the epitope alteration score and the semantic change score.

“Infectivity Score” or “Fitness Prior Score”: as used interchangeably herein, the term “infectivity score” or “fitness prior score” is a measure of a viral variant's evolutionary fitness, and is a function of the efficiency with which a virus replicates and/or the efficiency with which a virus infects host cells. In some embodiments, calculation of a fitness prior score comprises determining one or more of a log-likelihood score, a viral polypeptide receptor binding score, and/or a growth score. In some embodiments, a fitness prior score is determined by referencing each of a log-likelihood score, a viral polypeptide receptor binding score, and a growth score.

“Improve,” “increase”, “inhibit” or “reduce”: As used herein, the terms “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

In vitro: The term “in vitro” as used herein refers to events that occur in an artificial environment, e.g., in a test tube or reaction vessel, in cell culture, etc., rather than within a multi-cellular organism.

Log-likelihood: As used herein, the term “log-likelihood” refers to a measure of the existence probability of a variant polypeptide sequence, which has been determined using natural language learning algorithms. In some embodiments, log-likelihood can be determined using a transformer model. In some embodiments, log-likelihood can be determined without a reference sequence. In some embodiments, log-likelihood is a transformer-derived log-likelihood without reference. The higher the log-likelihood of a variant, the more probable the variant is to occur from a language model perspective. In various embodiments described herein, log-likelihood can be normalized such that it ranks between 0 and 100%. In some embodiments, a log-likelihood measures how log-likelihood of a variant polypeptide sequence compares to the entire population of known variants. In some embodiments, a log-likelihood measures how log-likelihood of a variant polypeptide sequence compares to other variants with similar mutational loads (“conditional log-likelihood”). Such conditional log-likelihood is particularly useful for assessing variants with high mutation counts (e.g., at least 30 or more, including, e.g., at least 40, at least 50, at least 60, at least 70, or more mutation counts).

Nucleic acid: As used herein, the term “nucleic cad” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Pareto Score: As used herein, the term “Pareto score” refers to a measure of a variant's fitness and ability to escape an immune response. In some embodiments, a Pareto score comprises a combination of an immune escape score (e.g., as described herein) and a fitness prior score (e.g., as described herein). In some embodiments, a Pareto score captures the relative evolutionary advantage of a given strain. In some embodiments, such a Pareto score can be determined as described in the Examples. In some embodiments, a Pareto score is an optimality score, which, for example in some embodiments ranks a variant relative to other sequences, e.g., ones that are observed in a population. A high Pareto score at a given time for a specific lineage indicates that fewer variants have higher scores for fitness prior and immune escape at that time. As a Pareto score in some embodiments is a ranking system, and fitness prior and immune escape scores incorporated therein can change as new data are acquired, the Pareto score for a given variant can change over time. As used herein, in some embodiments, Pareto optimality is defined over a set of lineages. In some embodiments, lineages are Pareto optimal within a set if there are no lineages in the set with higher immune escape and higher fitness prior scores. In some embodiments, a Pareto score is a measure of the degree of Pareto optimality. For example, in some embodiments, lineages with the highest Pareto score are Pareto optimal; and lineages with the second-best Pareto score would be Pareto optimal, if the Pareto optimal lineages were removed from the set, and so on.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes a viral infection (e.g., a SARS-CoV-2 infection). In some embodiments, the patient is receiving or has received certain therapy to diagnose and/or to treat a disease, disorder, or condition.

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g; replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Ribonucleic acid (RNA): As used herein, the term “RNA” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments where an RNA is an mRNA. In some embodiments where an RNA is an mRNA, an RNA typically comprises at its 3′ end a poly(A) region. In some embodiments where an RNA is an mRNA, an RNA typically comprises at its 5′ end an art-recognized cap structure, e.g., for recognizing and attachment of an mRNA to a ribosome to initiate translation. In some embodiments, an RNA is a synthetic RNA. Synthetic RNAs include RNAs that are synthesized in vitro (e.g., by enzymatic synthesis methods and/or by chemical synthesis methods). In some embodiments, an RNA is a single-stranded RNA. In some embodiments, a single-stranded RNA may comprise self-complementary elements and/or may establish a secondary and/or tertiary structure. One of ordinary skill in the art will understand that when a single-stranded RNA is referred to as “encoding,” it can mean that it comprises a nucleic acid sequence that itself encodes or that it comprises a complement of the nucleic acid sequence that encodes. In some embodiments, a single-stranded RNA can be a self-amplifying RNA (also known as self-replicating RNA).

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, spleen, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Semantic Change: As used herein, the term “semantic change” refers to a measure of a functional change of a viral polypeptide of a variant (e.g., in some embodiments a viral polypeptide that interacts with a host cell receptor and/or is otherwise involved in host cell entry) with respect to at least one or a plurality of (e.g., at least two, at least three, at least four, or more) reference viral polypeptide(s) (e.g., in some embodiments reference viral polypeptides of wild type species and/or known variants, e.g., of the same lineage) from the language model perspective. In some embodiments, a semantic change is a measure of a functional change of a viral polypeptide of a variant (e.g., in some embodiments a viral polypeptide that interacts with a host cell receptor and/or otherwise involved in host cell entry) with respect to a plurality of (e.g., at least two or more) reference viral polypeptide(s) (e.g., in some embodiments reference viral polypeptides of wild type species and/or known variants, e.g., of the same lineage) from the language model perspective. In some embodiments, a relevant language model can comprise Transformer-derived embedding differences (e.g., as described herein) with respect to at least one or a plurality of (e.g., at least two, at least three, at least four, or more) reference viral polypeptide(s) (e.g., in some embodiments reference viral polypeptides of wild type species or known variants, e.g., of the same lineage). In some embodiments, a semantic change score can be computed using L1 norm. In some embodiments, a sematic change score can be computed using L2 norm (also known as Euclidean norm). In some embodiments, semantic change describes how different a variant is with regard to an underlying statistical model (e.g., in some embodiments a large machine learning model fine-tuned on viral protein sequences observed until a given time point). In some embodiments, semantic change score depends on sequences observed, and thus the semantic change score may change over time, as an underlying model is trained on new variant sequences and/or reference sequences. In some embodiments, a semantic change score is determined for a variant Spike polypeptide from SARS-Co-V-2 as described herein. In various embodiments described herein, a semantic change score can be normalized such that it ranks between 0 and 100%.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial identity: as used herein, the term “substantial identify” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Threshold level: As used herein, the term “threshold level” refers to a level that are used as a reference to attain information on and/or classify the results of a measurement, for example, the results of a measurement attained in an assay. For example, in some embodiments, a threshold level means a value measured in an assay that defines the dividing line between two subsets of a population (e.g., a batch that satisfy quality control criteria vs. a batch that does not satisfy quality control criteria). Thus, a value that is equal to or higher than the threshold level defines one subset of the population, and a value that is lower than the threshold level defines the other subset of the population. A threshold level can be determined based on one or more control samples or across a population of control samples. A threshold level can be determined prior to, concurrently with, or after the measurement of interest is taken. In some embodiments, a threshold level can be a range of values.

Vaccination: As used herein, the term “vaccination” refers to the administration of a composition intended to generate an immune response, for example to a disease (e.g., to a viral epitope). In some embodiments, vaccination can be administered before, during, and/or after development of a disease. In some embodiments, vaccination includes multiple administrations, appropriately spaced in time, of a vaccinating composition.

Viral polypeptide receptor binding score: As used herein, the term “viral polypeptide receptor binding score” refers to a measure of binding affinity between a viral polypeptide that plays a role in host recognition and/or host cell entry, and a corresponding host protein with which the viral polypeptide interacts to recognize and/or enter a host cell. In some embodiments, a viral polypeptide receptor binding score is determined in silico. In some embodiments, a viral polypeptide receptor binding score can be determined using a conformational sampling algorithm. In some embodiments, a viral polypeptide receptor binding score can be determined using structures that have been optimized using a probabilistic optimization algorithm (for example, in some embodiments a variant of simulated annealing, aiming to overcome local energy barriers and follow a kinetically accessible path toward an attainable deep energy minimum with respect to a knowledge-based, protein-oriented potential). In some embodiments, a viral polypeptide receptor binding score can be calculated using the change in solvent accessible surface area (SASA) of a viral polypeptide in a complexed state (e.g., a bound state) and a non-complexed state (e.g., a non-bound state). In some embodiments, a viral polypeptide receptor binding score can be determined by calculating the change in energy of the complexed (e.g., bound) and non-complexed (e.g., non-bound) structures of a viral polypeptide and its cognate host receptor. In some embodiments, change in binding energy can be estimated by differences in Gibbs free energy between bound and unbound states. In various embodiments described herein, a viral polypeptide receptor binding score can be normalized such that it ranks between 0 and 100%. In some embodiments, a viral polypeptide receptor binding score can be calculated in silico, e.g., by calculating the change in Gibbs Free Energy, or the change in solvent accessible surface area in the bound and unbound states. In some embodiments, a viral polypeptide receptor binding score can be calculated using in vitro binding data (e.g., using a dissociation constant, K_{D}, or an association rate, kₒₙ). In some embodiments, such in vitro binding data can be determined methods known in the art, including, e.g., but not limited to biolayer interferometry (BLI) and/or surface plasmon resonance (SPR).

ACE2 Binding Score: As used herein, the term “ACE2 binding score” is a viral polypeptide receptor binding score (as described herein), wherein the viral polypeptide receptor is angiotensin-converting enzyme 2 (ACE2). An “ACE2 binding score” is a measure of binding affinity between an S protein of a coronavirus (e.g., SARS-CoV-2) or an immunogenic fragment of the S protein (e.g., the RBD domain) and the ACE2 protein. In some embodiments, an ACE2 binding score can be calculated in silico, e.g., by calculating the change in Gibbs Free Energy, or the change in solvent accessible surface area in the bound and unbound states. In some embodiments, an ACE2 binding score can be calculated using in vitro binding data (e.g., using a dissociation constant, K_{D}, or an association rate, kₒₙ). In some embodiments, such in vitro binding data can be determined methods known in the art, including, e.g., but not limited to biolayer interferometry (BLI) and/or surface plasmon resonance (SPR).

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild-type genes and polypeptides often exist in multiple different forms (e.g., alleles). In some embodiments, in the context of SARS-CoV-2, “wild-type” refers to the Wuhan (e.g., NCBI Ref.: 43740568) variant.

### DETAILED DESCRIPTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader—the presence and/or placement of a header is not intended to limit the scope of the subject matter described herein.

Described herein are technologies that facilitate identification, monitoring, and characterization of variants of infectious agents that pose elevated risk. In certain embodiments, technologies described herein include and/or may be implemented as a comprehensive research decision support system (e.g., a Digital Variant Research System (DVRS)) to, for example, assist stakeholders such as researchers, health system decision makers, officials, etc., in identifying and/or monitoring certain variants, such as potential variants of concern (VOC). For example, in certain embodiments, decision support technologies described herein can help health care decision makers make analytics-informed decisions on strategy for responding to infectious agents (e.g., pandemics). In certain embodiments, technologies of the present disclosure may facilitate informed and targeted vaccination strategies as well as timely development and production of modified vaccines, which can contribute to effective containment of local outbreaks.

For example, in certain embodiments, decision support systems of the present disclosure include and/or interface with an artificial-intelligence (AI)-based Early Warning System (EWS) that leverages available (e.g., worldwide) sequencing data to predict and/or rank potential high-risk variants of a particular reference infectious agent. In certain embodiments, an EWS may include a multi-layered, AI-based prediction model of pandemic potential (e.g., infectivity) and immune escape potential of new variants. Examples of EWS and computational approaches for immunogen selection and creation are described in further detail, for example, in (i) PCT Publication No. WO 2022/235847 A1, entitled “TECHNOLOGIES FOR EARLY DETECTION OF VARIANTS OF INTEREST,” and published Nov. 10, 2022, (ii) PCT Publication No. WO 2022/235853 A1, entitled “IMMUNOGEN SELECTION,” and published Nov. 10, 2022, and (iii) K. Beguir et al., “Early computational detection of potential high-risk SARS-CoV-2 variants,” Computers in Biology and Medicine, 155 (2023) 106618, the content of each of which is incorporated herein by reference in its entirety.

In certain embodiments, decision support technologies of the present disclosure include a forward-looking system that identifies sets of mutations that are likely to lead to immune escape or breakthrough infections. Predicting mutations that are, for example, likely to lead to new variants of concern and presenting them to users such as local health authorities and researchers, forward-looking systems thus may, for example, allow local health authorities and researchers to identify and monitor variants and lineages at an early stage, just as they begin to acquire mutations favorable to immune escape (e.g., even before a potentially serious variant has arisen or taken hold in a population).

In certain embodiments, the present disclosure provides for AI-assisted forecasting technologies that can be used to group variant lineages into clusters based on machine-learning model-computed representations that quantify similarities (e.g., underlying biophysical similarities, which impact immune-system recognition) between variants. As described in further detail herein, clustering variant lineages in this manner allows for more accurate and meaningful forecasting of distributions of viral variants at present and/or future time-points based on historical sequence data, than, for example, when variants are grouped according to conventional lineage schemes.

In certain embodiments, EWS-based predictions, as well as additional automated analyses, such as forward-looking mutation predictions, epitope conservation/alteration metrics, and lineage clustering and forecasting analyses are provided to a user via an interactive DVRS portal comprising one or more interactive GUI pages that users may navigate and view to monitor and gain insight about circulating variants, their evolution, and resultant and/or predicted impact on immune response and infectivity. DVRS systems and interactive portals described herein may be deployed locally and/or in a cloud-based manner (e.g., accessible via a web-application), accommodating a variety of research and/or public health infrastructures.

### A. PREDICTING IMMUNE ESCAPE AND INFECTIVITY

In certain embodiments, technologies of the present disclosure include in-silico techniques for predicting immune escape and/or infectivity of pathogens such as circulating viral variants. Immune escape and/or infectivity prediction technologies of the present disclosure may use and/or include machine learning models (e.g., language models) and/or various other computational models, such as epitope alteration score calculations, structural modeling, binding calculations (e.g., ACE2 binding scoring), and the like. Among other things, as described in further detail herein, these computational techniques may be used to determine, for a particular pathogen, scores that measure its fitness—i.e., ability to infect, and proliferate amongst, a host population and/or immune escape potential—i.e., potential to evade (e.g., pre-existing) host immune response. These computational techniques, and/or scores generated by them, may be used separately and/or in combination to generate relative rankings of circulating pathogens, such as viral variants. By scoring and/or ranking circulating pathogens in this manner, emerging variants of concern can be identified rapidly, allowing, for example, local health authorities to prepare public systems to minimize overall disease spread and/or its worst consequences, such as overburdened health systems and extensive casualties.

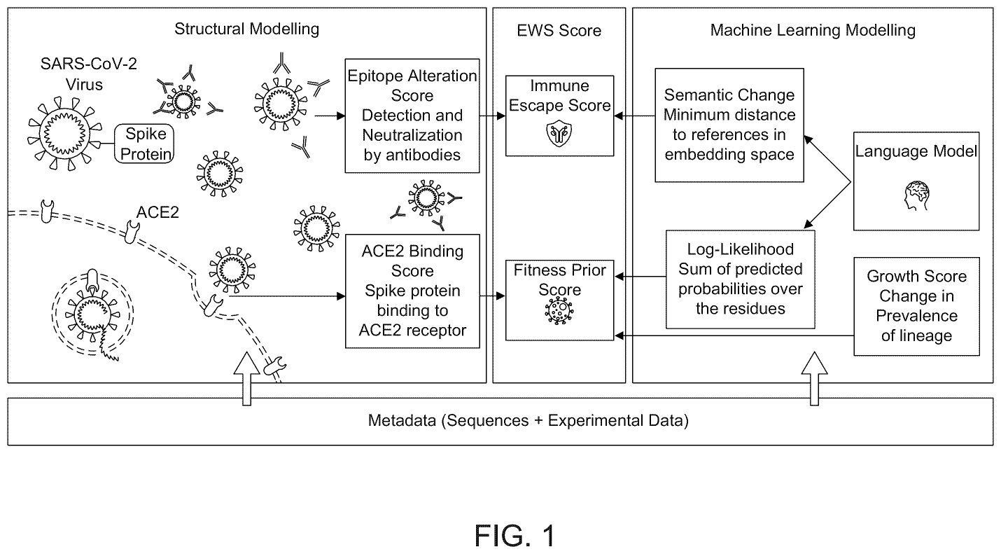

For example, FIG. 1 shows a schematic of an example AI-based prediction system providing early detection and identification of viral variants of concern—i.e., an Early Warning System (EWS)—which is described in further detail in (i) PCT Publication No. WO 2022/235847 A1, entitled “TECHNOLOGIES FOR EARLY DETECTION OF VARIANTS OF INTEREST,” and published Nov. 10, 2022, (ii) PCT Publication No. WO 2022/235853 A1, entitled “IMMUNOGEN SELECTION,” and published Nov. 10, 2022, and (iii) K. Beguir et al., “Early computational detection of potential high-risk SARS-CoV-2 variants,” Computers in Biology and Medicine, 155 (2023) 106618. EWS leverages in-silico structural modelling and machine learning (ML)-based language modelling to capture features of a given variant's fitness as well as its immune escape properties. In the context of the COVID-19 pandemic, analysis of in-silico predictions and data from experimental results about immune evasion and transmissibility have shown that EWS was able to identify high-risk SARS-CoV 2 variants about 10 to 50 days ahead of time of official declaration as VOC by the WHO. See, e.g., K. Beguir et al., “Early computational detection of potential high-risk SARS-CoV-2 variants,” Computers in Biology and Medicine, 155 (2023) 106618.

In particular, among other things, the example EWS shown in FIG. 1 utilizes machine learning modelling in combination with structural modelling to determine multiple scores representing individual infectivity (also referred to as fitness) potential and immune escape potential predictions. These scores may be used individually and/or in combination to determine, for example, an overall risk that each of one or more particular pathogens—e.g., as shown in FIG. 1, SARS-CoV-2 viral variants—represents in terms of pandemic potential and/or to identify particular pathogens—e.g., particular viral variants—of concern.

Infectivity potential predictions (e.g., infectivity potential, or fitness, scores) as determined and used by various technologies of the present disclosure may measure and/or rank, for each of a plurality of variants, likelihood of pathogen fitness (e.g., ability to infect host cells and/or proliferate). As described in further detail herein, infectivity potential predictions may comprise determining values of one or more infectivity score that measure various features contributing to and/or correlating with pathogen fitness and/or infectivity potential, such as abilities to enter host cells (e.g., as reflected via receptor binding calculations), statistical likelihood based on similarity to other viral variants (e.g., as measured via machine learning model predictions), and epidemiological data (e.g., growth statistics). In certain embodiments, various infectivity potential score values may be used to sort and/or rank a pool of observed variants, identifying a subset of variants with high infectivity potential.

Immune escape potential predictions (e.g., immune escape scores) may measure and/or rank, for each of a plurality of variants, a potential to evade host immune responses, such as neutralization by antibodies and T-cell recognition. As described in further detail herein, immune escape potential predictions may comprise determining values of one or more immune escape scores that measure various features contributing to and/or correlate with a potential to and/or extent to which a variant will evade host immune system response. Immune escape scores may be determined with reference to immune responses believed to be primed via exposure to one or more reference variants. For example, in the context of SARS-CoV 2, immune escape potential may be measured with reference to wild-type (e.g., NCBI Ref.: 43740568, Wuhan) virus, other previously circulating variants, and/or particular variants known to be included in vaccine compositions.

In certain embodiments, immune escape scores may include metrics such as distances from other reference variants in particular representation spaces (e.g., based on machine learning embedding representations, such as characteristic vectors determined from sequence embedding representations), measures of an extent to which particular epitopes targeted by host immune responses (e.g., by neutralizing antibodies and/or implicated in antigen presentation to T-cells) are altered in a particular variant polypeptide, and the like.

In certain embodiments, various immune escape potential score values may be used to sort and/or rank a pool of observed variants, identifying a subset of variants with high immune escape potential.

In certain embodiments, multiple infectivity scores may be combined to determine an overall infectivity score, such as a fitness prior score as shown in FIG. 1. In certain embodiments, multiple immune escape potential scores may be combined to determine an overall immune escape score. In certain embodiments, immune escape and infectivity scores may be combined to determine an overall risk score for each variant. In certain embodiments, immune escape and infectivity scores may be used to generate an AI-based prediction system determined list comprising a selected subset of a pool of variants that are identified (e.g., according to immune escape scores, infectivity scores, and/or combinations thereof) as posing an elevated risk (e.g., of pandemic potential), e.g., relative to other circulating variants.

i. Language Model-Based Predictions of Immune Escape and/or Infectivity

In certain embodiments, prediction systems of the present disclosure may utilize machine learning-based models. Machine learning models used in connection with technologies of the present disclosure may utilize and implement a variety of machine learning techniques. For example, a machine learning model may be a deep learning model (e.g., an artificial neural network with one or more, e.g., a plurality of, hidden layers), such as a language or large language model (LLM). In certain embodiments, a machine learning model is or comprises one or more recurrent models, such long short-term memories (LSTMs), implemented alone or in combination, e.g., as in a bi-directional LSTM (bi-LSTM). In certain embodiments, a machine learning model may be or comprise one or more transformer models. Examples of machine learning language models include, without limitation, evolutionary scale models (ESM), bidirectional encoder representations from transformers (BERT), and the like.

Machine learning language models may be trained, for example on protein sequence data available from public and/or private repositories, such as UniRef (see, e.g., Suzek et al. “UniRef: comprehensive and non-redundant UniProt reference clusters” 2007), the Virus Pathogen Resource (ViPR) database by the National Centre for Biotechnology Information, and the like. Training may be accomplished by providing a machine learning model with input sequences that are incomplete and/or partially masked and tasking them to predict the omitted and/or masked data. In this manner, machine learning models may be trained in an unsupervised fashion. Additional details of machine learning models and approaches for training them, such as particular techniques for training recurrent and/or transformer models, may be found, for example, in PCT publications WO 2022/235847 and WO 2022/235853, the content of each of which is incorporated by reference herein in its entirety.

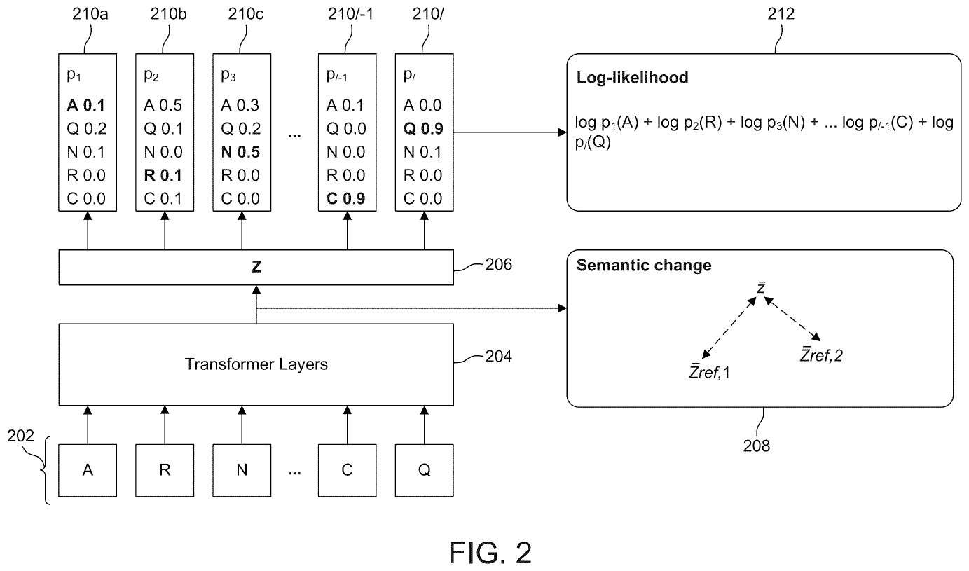

In particular, machine learning language models (also referred to as language models or large language models (LLMs)) may be used to generate predictions based on sequence data received as input. For example, FIG. 2 shows an example process 200 for computing certain fitness and immune escape scores using a language model that receives sequence data 202 for a particular pathogen. Sequence data 202 may be a biological sequence (e.g., an amino acid sequence and/or a nucleic acid sequence) of and/or encoding a polypeptide of a particular antigen of the pathogen, such as a SARS-CoV-2 spike (S) protein or portion thereof (e.g., a N-terminal domain, a receptor binding domain (RBD), etc.).

In some embodiments, an input sequence can be formally represented by a sequence of tokens defined as x=(x₁, . . . , xₙ) where n is the number of tokens and ∀i∈[1, n], xᵢ∈X where X is a finite alphabet that contains the amino-acids and other tokens such as class and mask tokens. In some embodiments, a class token is appended to all sequences before feeding them to the network, so that x₁ represents the class token, while x₂, . . . , xₙ represents the amino-acids, or masked amino-acids, in the spike protein sequence. In such embodiments, the sequence x is passed through attention layers. In these embodiments, z=(z₁, . . . , zₙ) corresponds to the output of the last attention layer where zᵢ is the sequence embedding vector at position i.

In some embodiments, embedding vector zᵢ is a function of all input tokens (xⱼ)_{j∈[1,n]}. In contrast, in Bi-LSTM architectures, such as described in Hie et al., 2021), zᵢ would be a function of all inputs tokens except the one at the position i, (xⱼ)_{j∈[1,n],j≠i}.

As shown in FIG. 2, a language model may be a transformer model comprising one or more (e.g., a plurality of) transformer layers 204. Transformer layers 204 of a language model may operate on a biological sequence 202 received as input and create a high dimensional representation of the input sequence 202, referred to as an embedding, z, 206. In some embodiments, to represent a protein sequence through a single embedding vector, whose size does not depend on the protein sequence length, the following equation can be used

$$\overline{z} = \frac{1}{n - 1} {\sum }_{i = 2}^{n} {z}_{i} ,$$

the product of which is referred to herein as the embedding or characteristic vector of the variant represented by sequence x. In the above equation, summation starts at the second position so that the class token's embedding, which is at the first position, does not contribute to the sequence embedding.

In certain embodiments, an embedding z 206 may be extracted (e.g., as output of a last transformer layer) and averaged over the residues to according to the equation above to obtain a characteristic vector of an input protein sequence 202. Where the protein sequence 202 is a sequence of a particular antigen, such as a viral variant polypeptide, it may be compared to characteristic vectors determined for one or more reference antigens, in order to determine a semantic change score 208. For example, as illustrated in FIG. 2, semantic change may be determined based on a measure of distance between (i) a characteristic vector for a particular antigen sequence (e.g., a particular variant of a viral protein or portion thereof) and (ii) characteristic vectors of one or more reference antigens (e.g., wild-type and/or selected reference variants of the same viral protein).

For example, in the context of SARS-CoV-2, a semantic change score for a particular selected variant may be determined for a sequence of that variant's SARS-CoV-2 (S) protein or a particular portion thereof, such as a receptor binding domain. The selected variant's characteristic vector may then be compared to characteristic vectors of one or more reference variants, such as a wild-type (e.g., NCBI Ref.: 43740568, Wuhan) strain and/or various selected reference strains, such as a D614G variant. For example, in some embodiments, the semantic change of a variant x can be computed as:

$$Δ \overline{z} = {‖ \overline{z} - {\overline{z}}_{wildtype} ‖}_{1} + {‖ \overline{z} - {\overline{z}}_{D 614 G} ‖}_{1} ,$$

where ∥ ∥₁ is the L1 norm. One of skill in the art, reading the present disclosure, will understand that while Wuhan (e.g., NCBI Ref.: 43740568) wild-type and D614G sequences are used as reference sequences in the above equation, other references sequences can also be used instead.

In some embodiments, the semantic change can be computed as the sum of the Euclidean distance between the z and z_{wildtype} the Euclidean distance between z and z_{D614G}. For example, in some embodiments, the semantic change of a variant x can be computed as:

$$Δ \overline{z} = {‖ z - {z}_{wildtype} ‖}_{2} + {‖ z - {z}_{D 614 G} ‖}_{2} ,$$

where ∥ ∥₂ is the Euclidean distance (also known as L2 norm).

In some embodiments, semantic change scores computed in this manner measure a similarity between a particular viral variant and one or more other, e.g., pre-existing, reference variants and, in turn, therefore reflect and provide a quantitative measure of how likely that particular variant is to be recognized by a host immune response developed in response to infection by the one or more reference variants. In this manner, semantic changes scores may provide a measure of immune escape potential.

In some embodiments, the log-likelihood can be computed from the probabilities over the residues returned by the model. In some embodiments, it is calculated as the sum of the log-probabilities over all the positions of the spike protein amino-acids.

For example, given a variant's sequence s, a network provides a discrete probability distribution over all amino acids A for each position i (e.g., 210a, 210b, 210c, . . . 210l-1, 210l in FIG. 2):

$${\{ p ( {s}_{i} = a \vert s ) \}}_{a ∈ A}$$

where p(sᵢ=a|s) is the probability that the i-th position is amino acid a. The variant's log-likelihood metric 212 is therefore defined as

$$l ( s ) = {\sum }_{i} log p ( {s}_{i} = {s}_{i} \vert s ) ,$$

which measures the likelihood of having the same variant given itself. In particular, the proposed log-likelihood metric supports substitution, insertion and deletion without requirement of a reference.

In some embodiments, the last attention layer output z can be transformed by a feed-forward layer and a softmax activation into a vector of probabilities over tokens at each positions p=(p₁, . . . , pₙ) where pᵢ is a vector of probabilities at position i, pᵢ=(p(xᵢ=x₁|x), . . . , p(xᵢ=xₙ|x)).

In some embodiments, the log-likelihood of a variant l(x) can be computed from such probabilities. In such embodiments, the log-likelihood can be calculated as the sum of the log probabilities over all the positions of the viral polypeptide amino acids (e.g., in some embodiments Spike protein amino acids). Formally, this can be written as:

$$l ( x ) = {\sum }_{i = 2}^{n} log p ( {x}_{i} = {x}_{i} \vert x ) .$$

This above equation measures the likelihood of observing a variant sequence x according to a model (e.g., as described herein) and, accordingly, provides a measure of fitness of the observed variant sequence. Therefore, the more sequences in the training data that are similar to a considered variant, the higher the log-likelihood of this variant will be. The proposed log-likelihood metric supports substitution, insertion, and deletion without the requirement of a reference.

ii. Structural Modelling of Immune Escape and/or Infectivity

In some embodiments, calculation of an immune escape score comprises calculation of an epitope alteration score, wherein the epitope alteration score is determined by identifying one or more sequence alterations in a variant polypeptide, and comparing the location and/or nature of the one or more sequence alterations to amino acid loci that have previously been shown to be bound by neutralizing antibodies. In some embodiments, the amino acid loci are determined using previously determined structures of the reference polypeptide in complex with neutralizing antibodies.

In some embodiments, an epitope alteration score described herein attempts to capture the impact of mutations in the variant in question on recognition by experimentally assessed antibodies. In some embodiments, an epitope alteration score can be computed by enumerating the number of unique epitopes involving altered positions, as measured across one or more known antibody-viral polypeptide complex structures (e.g., all known antibody antibody-Spike complex structures).

Without wishing to be bound by a particular theory, in some embodiments, an epitope alteration score as described herein is designed to emphasize the effect of mutations on highly antigenic sites of a viral polypeptide, such as in some embodiments the receptor-binding domain (RBD) of a Spike polypeptide. This allows the score to approximate the expected weight of mutations, and to ascribe importance to non-target domain mutations (e.g., non-RBD mutations), if sufficient escape potential with regard to targeting antibodies (e.g., RBD-targeting antibodies) is achieved.

In some embodiments, a viral polypeptide receptor binding score is a measure of the binding affinity between a viral polypeptide that plays a role in host recognition and/or host cell entry, and the corresponding host protein with which the viral polypeptide interacts to recognize and/or enter a host cell. By way of example only, in some embodiments, a viral polypeptide receptor binding score is or comprises an ACE2 binding score. In some embodiments, an ACE2 binding score is a measure of the binding affinity between the S protein or a portion of the S protein (e.g., the RBD domain) and the ACE2 protein. In some embodiments, an ACE2 binding score can be generated using a conformational sampling algorithm. In some embodiments, an ACE2 binding score can be generated using structures that have been further optimized using a probabilistic optimization algorithm, a variant of simulated annealing, aiming to overcome local energy barriers and follow a kinetically accessible path toward an attainable deep energy minimum with respect to a knowledge-based, protein-oriented potential.

In some embodiments, a viral polypeptide binding score can be calculated using the change in the surface accessible surface area (SASA) between the bound and the unbound structures of a viral polypeptide and a host protein. In some embodiments, the SASA measurements can then be aggregated per variant (e.g., RBD variant) using medians. In some embodiments, each metric can be normalized by the metric relative to a reference sequence (e.g., wild type sequence or an RBD sequence having no mutations), such that the binding score for the reference sequence is one.

In some embodiments, a viral polypeptide binding score can be calculated using the change in Gibbs free energy between the bound and unbound states, e.g., using the change in binding energy when the interface forming chains are separated, versus when they are complexed. In some embodiments, the binding energy measurements can be aggregated per variant (e.g., RBD variant) using medians. In some embodiments, each metric can be normalized relative to a reference sequence (e.g., a wild type sequence, corresponding to no mutation on target domain (e.g., RBDs)) such that the binding score for the reference sequence is one.

In some embodiments, variant sequences having combinations of mutations, representing very rare viral polypeptide, for example, in some embodiments corresponding to less than 10% of all known sequence, can be excluded from such binding score analysis. Without wishing to be bound by a particular theory, such exclusion can be useful to improve computational efficiency. By way of example only, in some embodiments, sequences having other RBD mutation combinations, representing very rare RBDS, corresponding to <9% of all known sequences, can be excluded from such binding score analysis.

In some embodiments, a growth score can be calculated using data provided in a publicly available database (e.g., GISAID metadata). In some embodiments, a growth score is calculated using recently submitted data. For example, in some embodiments, a growth score is calculated using data that have been submitted within the last 6 months (e.g., data that have been provided in the last 5 months, the last 4 months, the last 3 months, or the last two months, or within the last month). In some embodiments, a growth score is calculated using data that have been collected in the last eight weeks. In some embodiments, for each variant or lineage thereof, its growth can be calculated by determining its proportional change in a population of variants (e.g., among all submissions of sequences) over time (e.g., by comparing its proportion of the population at two points in time). By way of example only, in some embodiments, growth of a variant or lineage thereof can be calculated by the ratio of the proportion of the variant or lineage thereof determined over a recent time window (e.g., within last week), rₗₐₛₜ, to the proportion observed over a more extended time window (e.g., a time window that goes beyond the recent time window, e.g., an eight-week window), r_{win}. The ratio of rₗₐₛₜ/r_{win} is a measure of the change of the proportion. Ratio values larger than one indicate that the variant or the lineage thereof is rising, and ratio values less than one indicate that the variant or lineage thereof is declining.

In some embodiments, an infectivity score (also known as a fitness prior score) described herein can reference a combination of a viral polypeptide receptor binding score and a log likelihood score. In some embodiments, an infectivity score (also known as a fitness prior score) described herein can reference a combination of a viral polypeptide receptor binding score, a log likelihood score, and a growth score. In some embodiments, experimental data (including, e.g., in vitro data) can be used to validate an infectivity score. For example, in some embodiments, binding affinity analysis between a target variant polypeptide and a cognate viral polypeptide receptor (e.g., RBD: ACE2 affinity analysis) can be performed to validate infectivity/transmissibility metric. Such affinity analysis can be performed using in vitro data that are already available and/or based on wet lab experiments using recombinant constructs of target polypeptides (e.g., RBD) from variants being assessed.

iii. Ranking Variants of Concern and Watch Lists

In some embodiments, to make the semantic change, log-likelihood, epitope score, viral polypeptide receptor binding score (e.g., ACE2 binding score) and growth rate capable of being compared directly, a scaling strategy is introduced. For a given metric m, all the variants considered can be ranked according to this metric. In the ranking system used, the higher rank the better. In some embodiments, variants with the same value for metric m will get the same rank. In some embodiments, the ranks are then transformed into values between 0 and 100 through a linear projection to obtain the values for the scaled metric m_{scaled}. In some embodiments, all computed scores can be scaled as described herein. In some embodiments, all computed scores, except for log-likelihood, can be scaled, for example, in some embodiments where variants may have a large number of mutations, e.g., more than 30 mutations, more than 40 mutations, more than 50 mutations, more than 60 mutations, more than 70 mutations, or higher. In some embodiments, log-likelihood may penalize variants with a large number of mutations. Without wishing to be bound by any particular theory, an increased number of mutations may impact fitness, explaining the decreased log-likelihood. However, given that variants scored using methods and/or systems disclosed herein have been registered, this suggests that they have managed to infect hosts and replicate sufficiently to be detected, and that they have at least minimal fitness. By way of example only, a variant with two mutations, whose log-likelihood is in the bottom 20^{th} percentile globally, may be less likely to survive evolutionary competition, while a variant, with analogous log-likelihood, but with twenty mutations may be more likely to survive evolutionary competition as compared to similarly mutated variants. In some such embodiments, a conditional log-likelihood score is introduced such that the log-likelihood of variants having high mutational load is ranked relative to other variants with a similar mutation rate, as opposed to rank them across all variants. Thus, in some embodiments, a group-based ranking strategy can be used, where each variant is ranked among variants with a similar number of mutations (e.g., within 10% difference). For example, in some embodiments, for each variant, having N mutations, its log-likelihood score is ranked among all variants having at least M mutations, wherein M is the less of an arbitrary value (e.g., about 100, about 75, about 50, or about 25) and N minus 0 to 20 (e.g., N minus about 5 to 15, or N minus about 10). In some embodiments, M=min(max(0, N−10), 50). In some embodiments, N-terminal and C-terminal deletions are considered as a single mutation for grouping purposes. In some embodiments, for each group, the ranks are then transformed into values between 0 and 100 through a linear projection to obtain the values for the scaled metric. In some embodiments, results may be largely robust to the choice of a threshold.

In some embodiments, an immune escape score is computed as an average of the scaled semantic change and of the scaled epitope score. In some embodiments, the infectivity score is computed as the sum of the scaled log-likelihood, the scaled viral polypeptide receptor binding score (e.g., ACE2 binding score) and the scaled growth rate. In some embodiments, the infectivity score is computed as the sum of the scaled conditional log-likelihood, the scaled viral polypeptide receptor binding score (e.g., ACE2 binding score) and the scaled growth rate.

In some embodiments, an immune escape score (e.g., as described herein) and a fitness prior score (e.g., as described herein) can be combined to yield a Pareto score. In some embodiments, a Pareto score is based on Pareto optimality. In some embodiments, Pareto optimality is defined over a set of lineages. In some embodiments, lineages are Pareto optimal within a set if there are no lineages in the set with both higher immune escape and higher fitness prior scores. In some embodiments, a Pareto score is a measure of the degree of Pareto optimality. Lineages with the highest Pareto score are Pareto optimal. Lineages with the second-best Pareto score would be Pareto optimal, if the Pareto optimal lineages were removed from the set, and so on.

In some embodiments, a Pareto score can be determined by computing all the Pareto fronts that exist in a considered set of lineages. By way of example only, the first Pareto front corresponds to a set of lineages for which there does not exist any other lineage with both higher immune escape and fitness prior score. The second Pareto front is computed as the Pareto front over the set of lineages remaining when removing the ones from the first Pareto front. Successive Pareto fronts are computed until all the lineages are assigned to a front. In some embodiments, a linear projection can be used so that the lineages from the first front obtain a Pareto score of 100 and the ones from the last front get a Pareto score of 0.

In some embodiments, a variant polypeptide is designated as elevated risk when (a) it has an immune escape score that satisfies a pre-determined immune escape threshold indicating likelihood of the variant polypeptide to be detected and neutralized by antibodies; and/or (b) it has an infectivity score that satisfies a pre-determined infectivity threshold indicating likelihood of the variant polypeptide to a relevant host receptor.

In some embodiments, a variant polypeptide is designated as elevated risk when (a) it has an immune escape score that satisfies a pre-determined immune escape threshold indicating likelihood of the variant polypeptide to be detected and neutralized by antibodies; and/or (b) it has an infectivity score that satisfies a pre-determined infectivity threshold indicating likelihood of the variant polypeptide to a relevant host receptor.

In some embodiments, a variant polypeptide is designated as elevated risk when (a) it has an immune escape score that is higher than the immune escape score of other variant polypeptides that are prevalent at the time of assessment (e.g., a score that is in the top 50% of sequences assessed, 40% of sequences assessed, 30% of sequences assessed, 20% of sequences assessed, 15% of sequences assessed, 10% of sequences assessed, or 5% of sequences assed), and/or (b) it has an infectivity score that is higher than the infectivity score of other variant polypeptides that are prevalent at the time of assessment (e.g., a score that is in the top 50% of sequences assessed, 40% of sequences assessed, 30% of sequences assessed, 20% of sequences assessed, 15% of sequences assessed, 10% of sequences assessed, or 5% of sequences assed), and/or a combination of immune escape score and infectivity score that is higher than those of other variant polypeptides that are prevalent at the time of assessment (e.g., a combined score that is in the top 50% of sequences assessed, 40% of sequences assessed, 30% of sequences assessed, 20% of sequences assessed, 15% of sequences assessed, 10% of sequences assessed, or 5% of sequences assed). In some embodiments, each of the variant polypeptides in a plurality of polypeptides share an overall amino acid sequence identity of at least 80% with each other (e.g., at least 85%, at least 90%, at least 95%, at least 96%, at least 97%, at least 98%, or at least 99% with each other). In some embodiments, each of the variant polypeptides in a plurality of polypeptides have an overall amino acid sequence identity of at least 80% with a reference polypeptide (e.g., at least 85%, at least 90%, at least 95%, at least 96%, at least 97%, at least 98%, or at least 99% with the reference polypeptide). In some embodiments, variant polypeptides that have been designated as having an elevated risk using technologies described herein are considered as “High Risk Variants” (HRV). In some embodiments, variant polypeptides that have been designated as having an elevated risk using technologies described herein are considered as “Variants of concern” (VOC). In some embodiments, variant polypeptides that have been designated as having an elevated risk using technologies described herein are considered as “Variants of Interest” (VOI). In some embodiments, variant polypeptides that have been designated as having an elevated risk using technologies described herein are considered as “Variants under Monitoring” (VUM).

Further details of exemplary approaches for scoring and ranking variants, including viral variants such as SARS-CoV-2 are described in in (i) PCT Publication No. WO 2022/235847 A1, entitled “TECHNOLOGIES FOR EARLY DETECTION OF VARIANTS OF INTEREST,” and published Nov. 10, 2022, (ii) PCT Publication No. WO 2022/235853 A1, entitled “IMMUNOGEN SELECTION,” and published Nov. 10, 2022, and (iii) K. Beguir et al., “Early computational detection of potential high-risk SARS-CoV-2 variants,” Computers in Biology and Medicine, 155 (2023) 106618, the contents of each of which is incorporated by reference herein in its entirety.

### B. PREDICTING IMMUNE ESCAPING MUTATIONS VIA A FORWARD-LOOKING SYSTEM

In certain embodiments, technologies of the present disclosure include a forward-looking system providing predictions, for a given lineage of a virus (e.g., SARS-CoV-2), which mutations are likely to increase immune evasion.

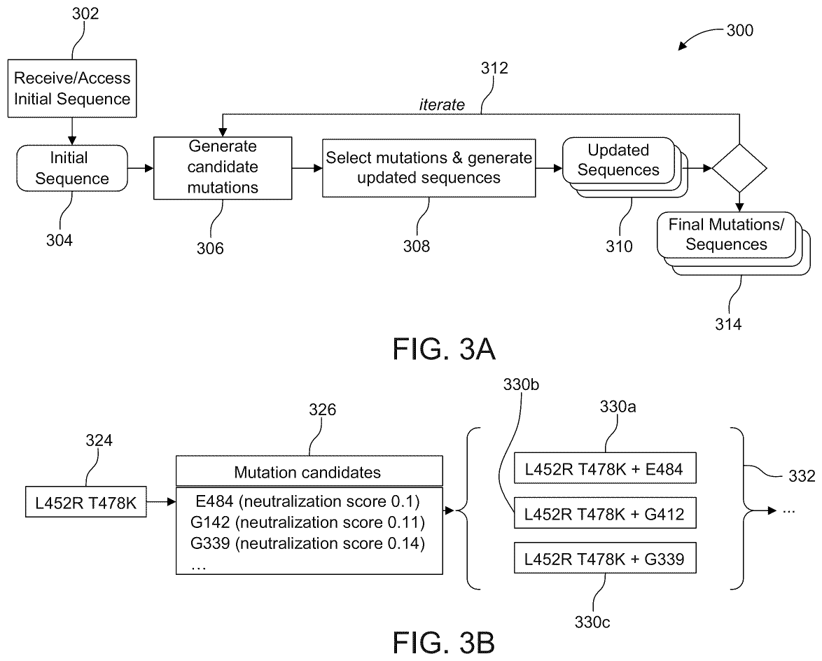

Turning to FIG. 3A, in an example process 300 for generating forward-looking predictions, a forward-looking system receives 302 a particular lineage (and its sequence) of a viral variant polypeptide 304, as input and provides, as output, a list of predicted mutations 314 that are identified as likely to increase immune evasion. For example, forward-looking system (FLS) process 300 may receive a sequence of a SARS-CoV-2 Spike (S) protein, or portions thereof—such as a receptor binding domain (RBD) and/or an n-terminal domain (NTD)—and provide an output list of NTD and/or RBD mutations. Without wishing to be bound to any particular theory, currently, RBD and NTD are believed to be main domains targeted by antibodies. In certain embodiments, for example, with regard to T-cell responses, not only Spike, but other non-Spike proteins such as membrane protein, nucleoprotein, NSP1-NSP4 may also be of interest and included in a FLS model.

In certain embodiments, FLS process 300 may be designed to account B-cell responses. In certain embodiments, FLS process 300 may be designed to account B-cell responses and T-cell responses.

In certain embodiments, a FLS process 300 may be used to predict mutations for multiple viral variants. For example, in certain embodiments, circulating viral variants may be evaluated using immune escape scores of an EWS system. Values of an immune escape score computed for circulating variants may be used to identify a subset of the variants for further monitoring. For example, circulating variants may be ranked according to their immune escape score values, and a subset having highest immune escape score values selected for further monitoring and/or analysis.

For example, a top-20 immune escaping variants may be identified and flagged for monitoring. FLS process 300 may then be used to predict, for each of the top-20 immune escaping sequences, a combination of predicted mutations that (e.g., where they to occur) would be likely to increase immune-escape potential of the virus.

FLS process 300 may, in certain embodiments, generate and score a plurality of candidate mutation combinations. A final set of predicted mutations may then be selected based on computed scores, which aim to measure a likelihood that a particular combination of mutations will increases immune evasion potential.

For example, in certain embodiments, FLS process uses a neutralization score to measure impact of a particular combination of mutations on immune escape. In certain embodiments, a neutralization score may be calculated using the following formula:

$${log}_{2} [ {\sum }_{i} {c}_{i}^{0} exp ( - {ϕ}_{i} ) ]$$

where cᵢ⁰ is a weight per epitope and ϕᵢ is a number of positions mutated on epitope i, as compared to particular (e.g., selected) reference antigen. In the context of a viral antigen, such as SARS-CoV-2, a selected reference antigen may be wild-type (e.g., NCBI Ref.: 43740568, e.g., Wuhan for SARS-CoV-2) or a particular variant (e.g., Alpha for SARS-CoV-2). In certain embodiments, a choice of reference antigen is based on an input vaccination/infection selection.

Without wishing to be bound to any particular theory, the particular functional form of the neutralization score shown above was designed based on the rationale that, given a particular epitope (a set of amino acids on an antigen), mutating these positions is assumed to be impacting (e.g., the ability/strength) of antibody binding thereto, and, accordingly, allow the antigen/virus to evade immune response. The particular embodiment shown in the equation above does not differentiate between different (types of Amino Acid) mutations at a same position and is representative that in general mutated sequences are considered to be more immune escaping. The particular functional form was designed to match available assay data.

In certain embodiments, epitope weights cᵢ⁰ are determined/adjusted to model a particular vaccination/breakthrough infection condition, such that a neutralization score value computed for a particular mutation will vary depending on a particular selected vaccination/breakthrough condition. For example, since different vaccination/breakthrough infection conditions are expected to produce different antibody responses, as well as T-cell and B-cell immunity, these, in turn, will vary the particular epitopes and/or their significance dictating host immune response. In certain embodiments, epitope weights can be varied to account for waning immunity (e.g., antibody immunity over time). For example, at a first exposure via infection or vaccination, a set of epitopes will be identified and considered as “elicited”. Each epitope can be assigned a weight, and over time weights can be gradually decreased, simulating antibody waning. Following, further infection or vaccination, potentially new epitopes can be included, and existing epitopes will be recalled. Accordingly, epitope weights can be updated to simulate ‘boosted’ response.

Epitope weights can be determined via a variety of experimental and/or in-silico techniques. For example, in certain embodiments, epitope weights can be determined using experimental assay data. Experimental assay data may include, without limitation, data from assays to measure antibody bind affinity and avidity through techniques, such as modified ELISA, solid phase radioimmunity assays, ammonium sulphate precipitation, surface plasmon resonance. In certain embodiments, experimental assay data may include functional assays such as a plaque reduction neutralization test (PRNT) or ELISpot. In certain embodiments experimental assay data maybe include identification of antibodies by single-cell V(D)J sequencing of antigen-specific memory B cells from individuals who had been infected with a pathogen or vaccinated against a pathogen. In certain embodiments, a set of hyper parameters is used to translate experimental data to epitope weights. In certain embodiments, machine learning models may be trained (e.g., using experimental assay data described herein, other experimental assay data, and/or data derived therefrom) and used to generate epitope weights.

Accordingly, neutralization scores calculations can be tailored, e.g., via the set of particular epitopes considered and/or their weights, to account for the unique immune response and, accordingly, resultant impact on particular viral mutations to evade it. For example, various potential vaccination schemes and/or breakthrough conditions, along with associated FLS system predictions are shown and described in Example 1, below. FLS processes can, accordingly, be tailored and/or adjusted over time and/or at different locations, to account for variations in population immunities over seasons, courses of pandemics, as new vaccinations are developed and adopted, with geography, etc.

In certain embodiments, FLS process 300 generates and scores candidate mutation combinations in an iterative fashion, as shown in FIG. 3A. For example, following receipt 302 of an initial sequence of a variant polypeptide 304, a plurality of candidate mutations are generated 306. In certain embodiments, each candidate mutation identifies a particular position within the variant polypeptide as a mutated position. Each candidate mutation is appended to an initial list of mutation that the initial, starting variant polypeptide comprises, so as to create a plurality of candidate mutation combinations. Values of a neutralization score may then be computed for each candidate mutation combination and used to select a subset of the candidate mutation combinations to retain 310. These steps may then be repeated 312, with each retained mutation combination used as a starting point for a subsequent step, adding candidate mutations and then scoring and selecting new mutation combinations to successively grow the lists of mutations defining each candidate mutation combination until one or more final sets 314 of mutation combinations are obtained at a final step.

For example, as shown in FIG. 3B, an initial starting sequence may include an initial set of mutations 324. Various mutation candidates 326 may be scored, and a subset, e.g., having lowest neutralization score values, selected and appended to the initial set of mutations 324, to generate lists of first predicted mutation combinations 330a, 330b, 330c. Each list of first predicted mutation combinations may be then passed to a second step 332, and so one, thereby growing a list of predicted mutations.

In certain embodiments, a FLS process selects and iterates through multiple steps using a modified beam search. In this approach, beginning with a particular starting sequence and vaccination/breakthrough condition, at each step: (i) an impact of each possible candidate mutation one immune escape is evaluated via the neutralization score described herein and (ii) a ‘best’ N candidates, identified based on their neutralization score values (e.g., those having lowest neutralization score values) are selected. These steps may be repeated multiple, e.g., M, times to generate N sets of predicted mutations, each comprising M new mutations (e.g., and, hence, N new variants, each having M added mutations). Variables N and M may be integers—for example, N may be 4, 8, 16, 32, 64, 128, etc. and M may be 2, 4, 8, 16, 32, etc.

In certain embodiments, mutations evaluated and generated via FLS processes described herein identify particular positions in an amino acid sequence to be mutated but need not necessarily identify a particular new amino acid (e.g., to be substituted at the mutated position). For example, in certain embodiments, identifying and scoring based on presence of mutations in epitopes (but not necessarily type) may be accomplished via approaches such as the neutralization score described herein.

In certain embodiments, candidate mutations are limited to those mutations that have been observed at least a threshold number of times (e.g., 5, 10, 15, 20, 50, 100, etc.) in a particular dataset, such as GSAID data, as a proxy for viral fitness. In certain embodiments, an entire dataset—not necessarily limited to a particular lineage or sub-lineage, is considered. In certain embodiments, all potential mutations may be considered. In certain embodiments, a machine learning approach, such as a language model may be used to propose mutations.

In this manner, FLS technologies described herein may provide insight into which mutations may be particularly concerning and likely to increase viral immune escape potential. In certain embodiments, new sequence submissions may be (e.g., regularly) monitored for presence of mutations predicted by FLS technologies described herein.

In certain embodiments, neutralization scores may consider biological and structural considerations, such as particular amino acid substitutions. In certain embodiments, fitness metrics, such as log-likelihood calculations may be used to account for amino acid types in substitution mutations when selecting candidate mutations and ensure viable substitutions.

In certain embodiments, datasets used by the FLS process may be updates regularly as well, e.g., to ensure epitopes considered and/or candidate mutations considered reflect current information and status of a circulating virus. For example, in certain embodiments, epitope data used by the FLS is updated regularly (monthly) and the data used to perform prediction is updated more frequently (weekly). In certain embodiments, updates to datasets do not require changes to the underlying method.

Parameters of FLS, such as minimum threshold observations, N, and M, may be adjusted to tailor predictions.

In providing insight into potentially concerning mutations, in certain embodiments, FLS tools and their predictions as described herein may compared with observed potentially concerning sequences, e.g., to identify potential mutations of concern. In certain embodiments, FLS as described herein can be used as a search/design system based on a set of scorers which are being used to evaluate reported sequences.

### C. GRAPHICAL INTERFACE(S) FOR DECISION SUPPORT SYSTEMS

In certain embodiments, AI-based prediction system data may be used in connection with a graphical user interface of a decision support system, e.g., to present users such as laboratory researchers, local health authorities, government officials and policymakers, and the like, with an interactive visual dashboard that provides and/or contextualizes predictions generated via AI-based prediction systems. For example, in certain embodiments, infectivity and/or immune escape data for a one or more (e.g., a plurality of) of variants may be rendered and displayed to a user via a GUI.

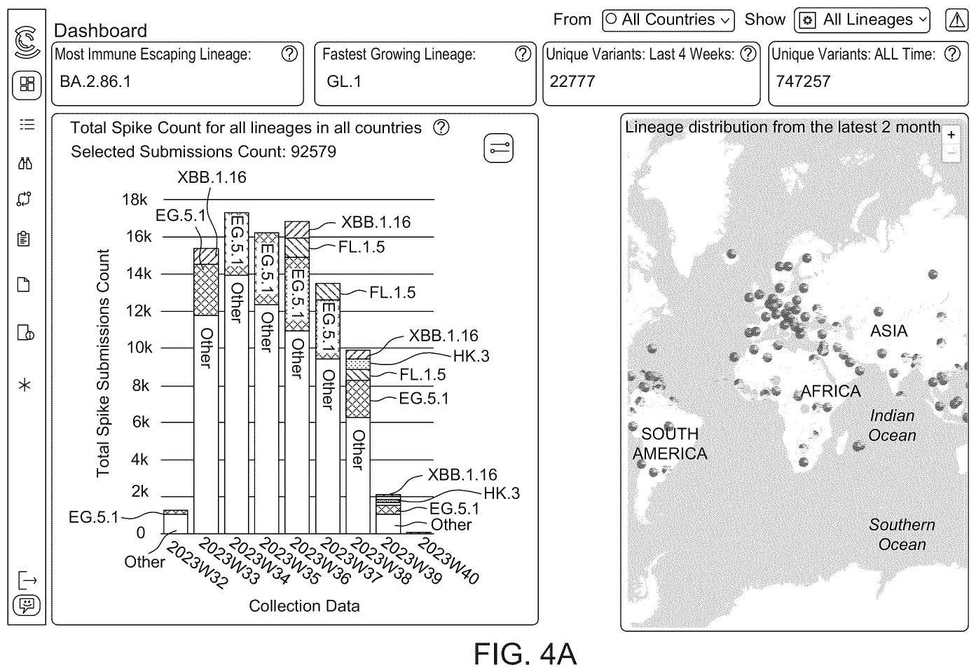

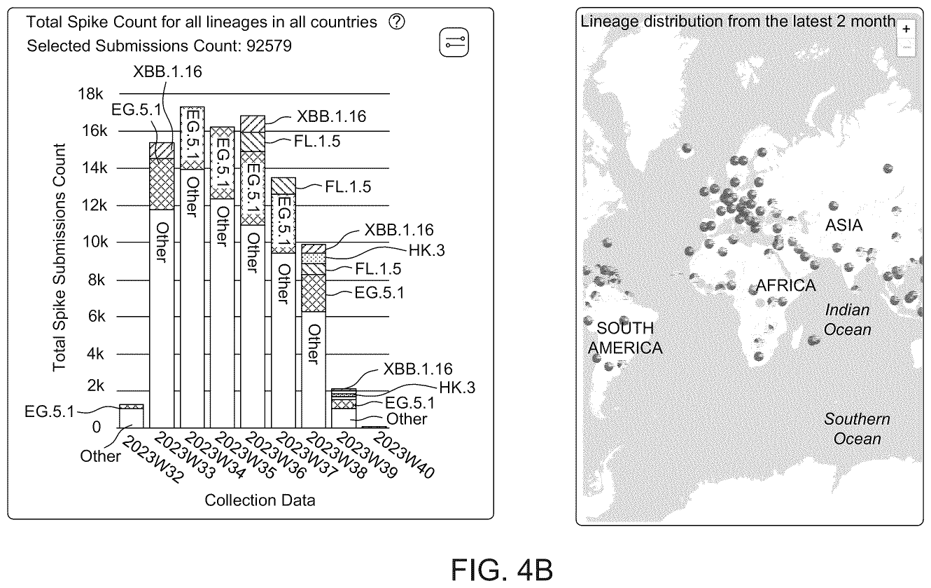

FIG. 4A shows a screenshot of an example dashboard page of an example DVRS, displaying information such as an identification of a most immune escaping variant, a fastest growing lineage, and numbers of unique variants observed over various time windows (e.g., a last four weeks and all time (e.g., to-date)). Example dashboard page shown in FIG. 4A includes panels displaying graphs, such as a bar graph showing breakdown amongst various lineages observed at various collection dates and a map with pie-chart icons placed at various locations (e.g., cities) and conveying relative fractions of various lineages at the locations where they are placed. An expanded view of the two such graphs from FIG. 4A is shown in FIG. 4B.

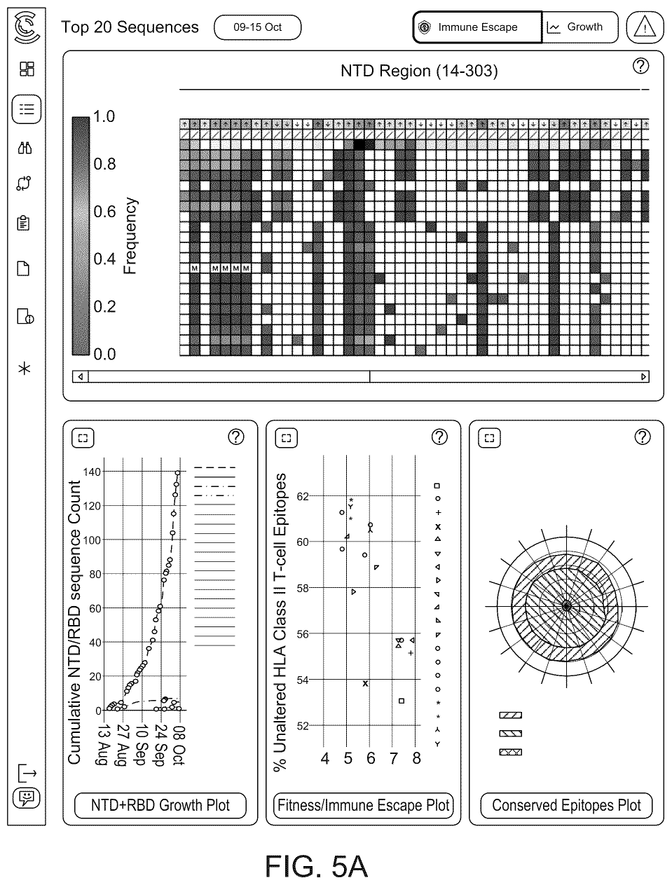

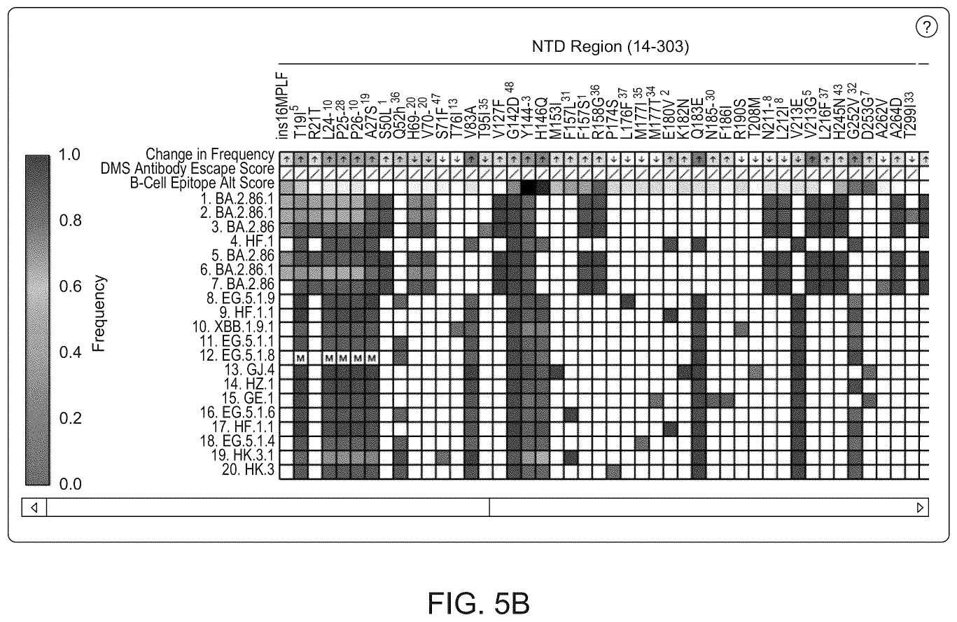

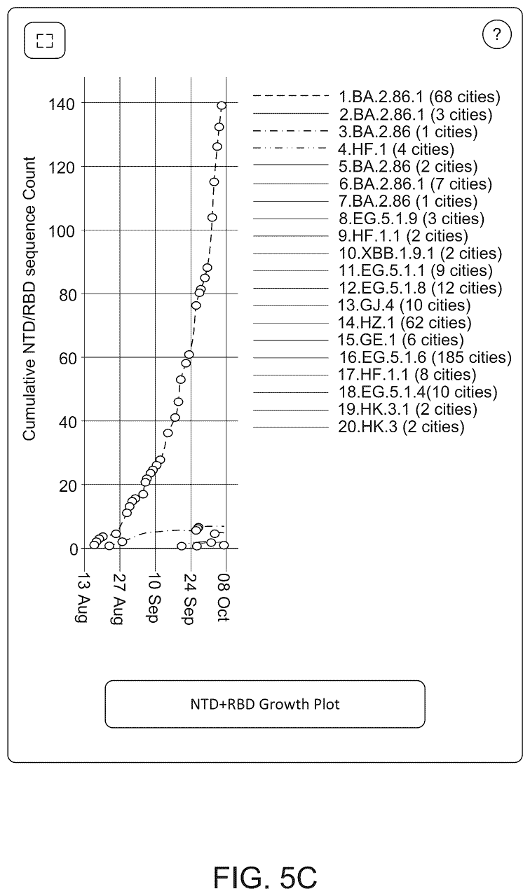

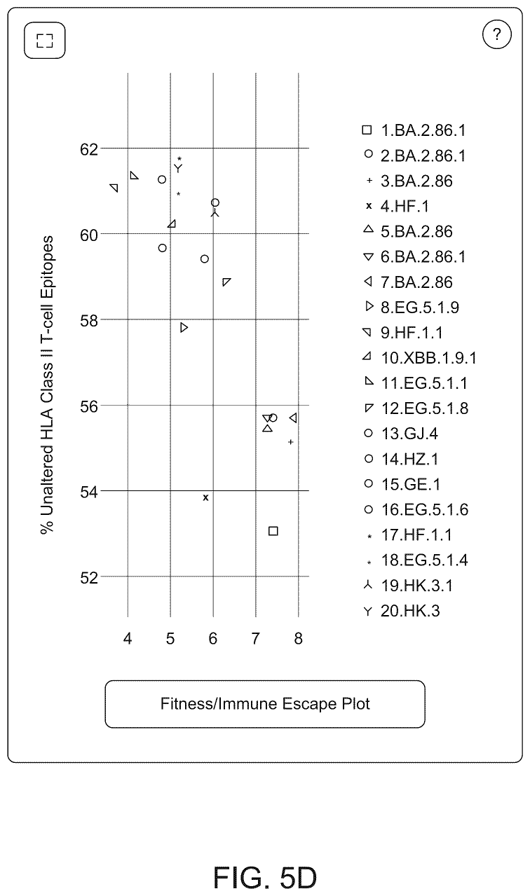

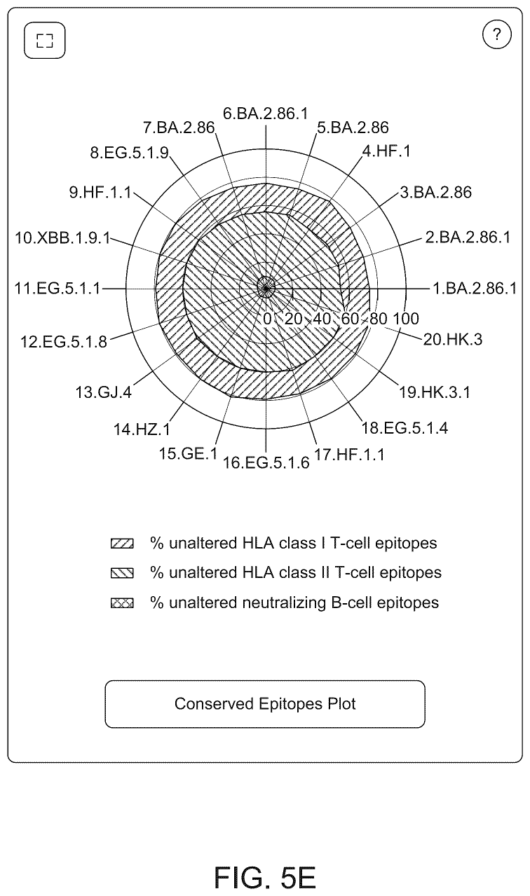

FIG. 5A shows a screenshot of an example GUI for monitoring immune escape and/or infectivity potential of a predetermined number (e.g., 20) of variants identified as having a highest immune escape potential. Example GUI shown in FIG. 5A includes a heatmap chart (expanded view shown in FIG. 5B) that lists mutations along a horizontal top axis and the top 20 variants along the vertical axis, such that each mutation is in a column and each variant is in a row. Each cell representing a particular variant-mutation combination is color coded according to a frequency of that mutation occurring in the particular variant. Example GUI shown in FIG. 5A also includes panels displaying graphs showing sequence growth and NTD plus RBD sequence growth, as well as immune escape data providing a visual display of HLA class I T-cell epitopes, HLA class II T-cell epitopes, and neutralizing B-cell epitopes, for a plurality of SARS CoV 2 variants, as shown in expanded views in FIGS. 5C-E.

As explained in further detail in A. Muik et al., “Progressive loss of conserved spike protein neutralizing antibody sites in Omicron sublineages is balanced by preserved T-cell recognition epitopes,” bioRxiv, posted Dec. 15, 2022, without wishing to be bound to any particular theory, while variants such as XBB may include several mutations in B-cell epitope that result in evasion of neutralizing antibodies, a large fraction of T-cell epitopes may continue to be conserved (i.e., unaltered) and confer additional immune defense, e.g., against severe disease. Accordingly, monitoring T-cell epitopes, additionally or alternatively with B-cell epitopes, may provide additional insight and context for decision makers with regard to resource allocation and response planning. Approaches described herein may, for example, obtain (e.g., receive and/or access) epitope data identifying B-cell neutralizing antibody epitopes and/or T-cell (HLA class I and/or class II) epitope data and (e.g., automatically) analyze sequence data to identify, for each of a plurality of viral variants, which epitopes are altered. Results of such analysis may be displayed, compared with AI-based prediction system results, such as immune escape scores, and/or provided as feedback (e.g., to an AI-based prediction system).

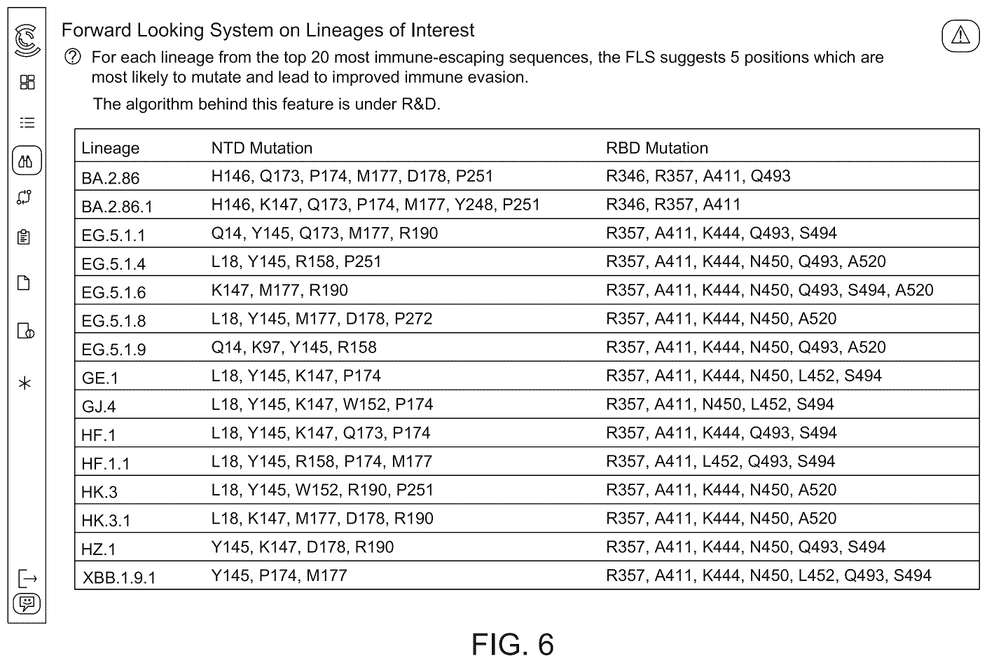

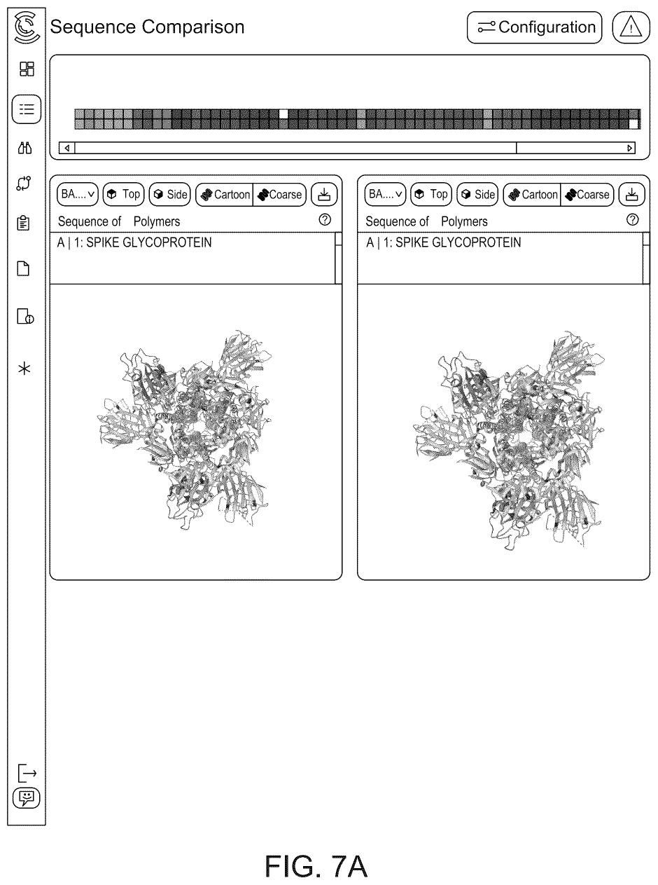

FIG. 6 shows a screenshot of a GUI page for displaying results of FLS predictions described herein. In example GUI shown in FIG. 6, lineages and predicted mutations are displayed in a tabular format, with each lineage occupying a row, and columns showing, for each lineage, a list of predicted mutations in NTD and RBD regions.

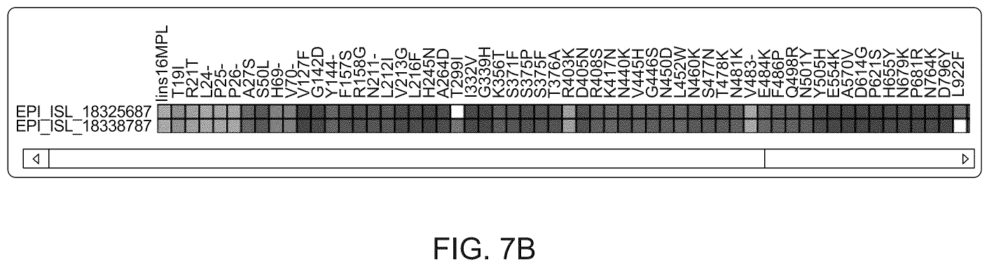

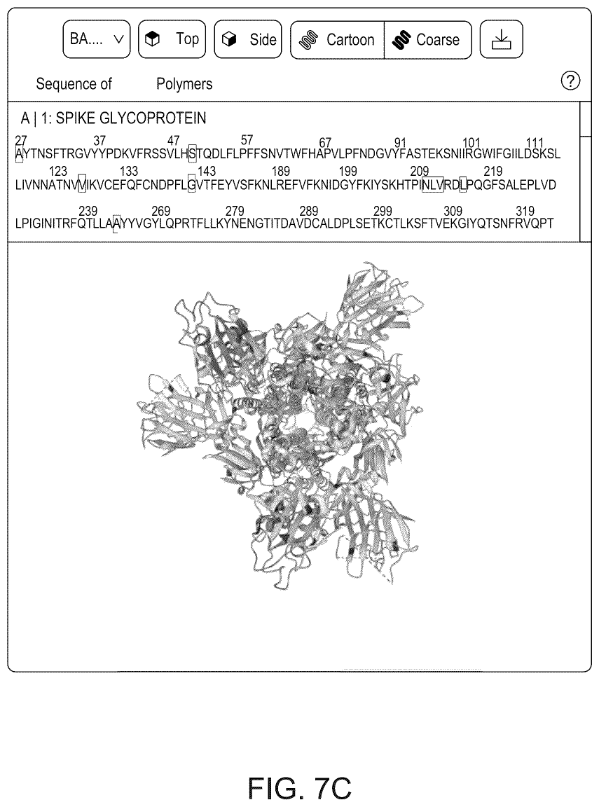

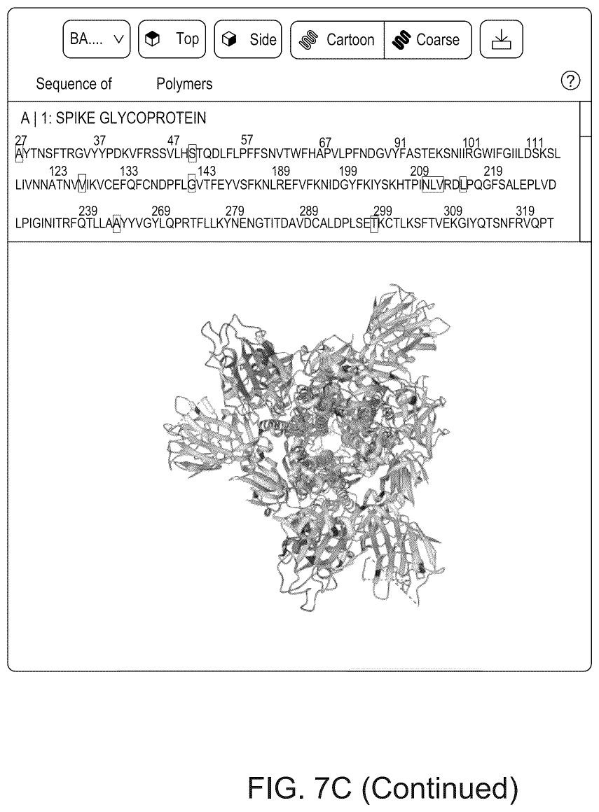

FIG. 7A shows a screenshot of an example GUI page whereby a user may compare two selected sequences. Example GUI page shown in FIG. 7A includes a heatmap (expanded view shown in FIG. 7B) for comparing frequencies of mutations in the two selected sequences, a text representation of each sequence, and a 3D structural viewer showing a 3D structure of the two polypeptides corresponding to each sequence (expanded view shown in FIG. 7C).

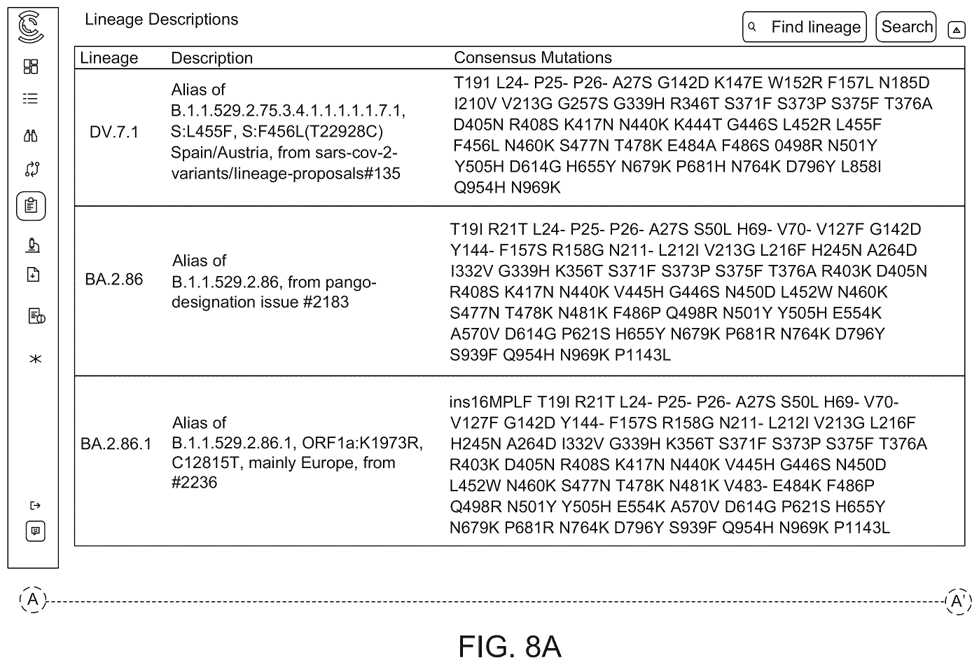

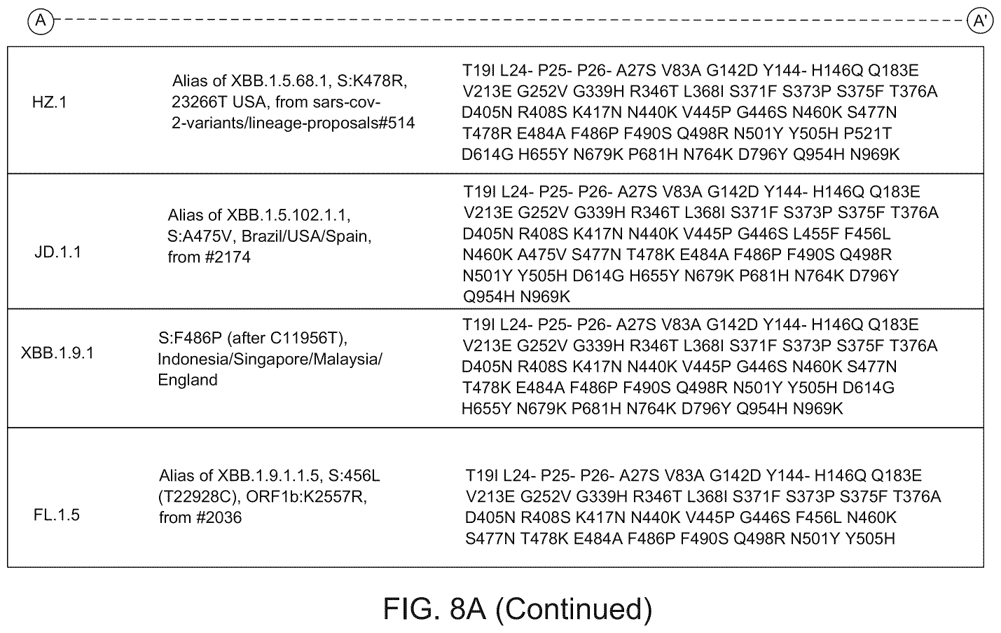

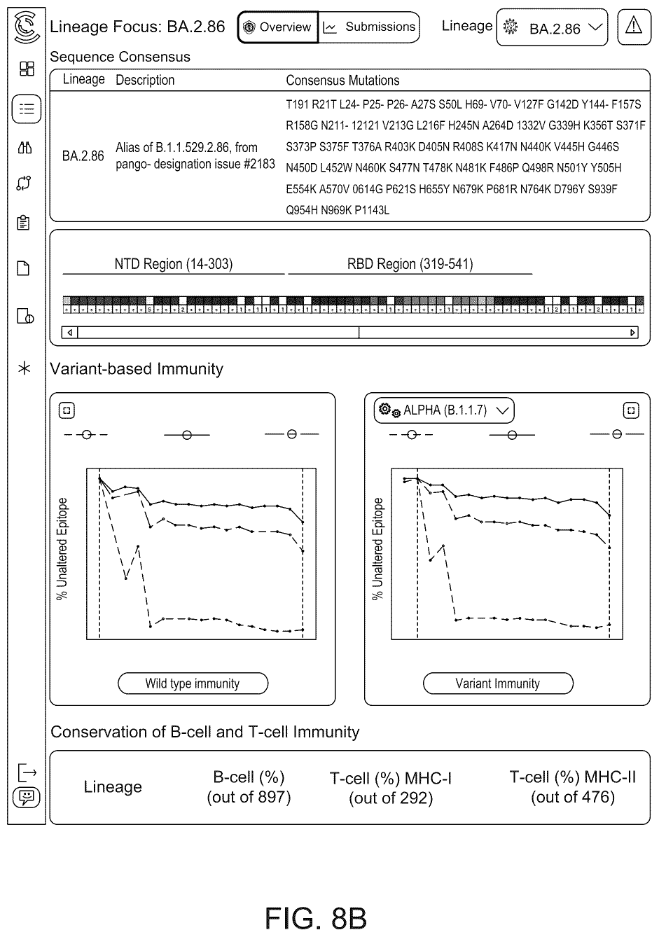

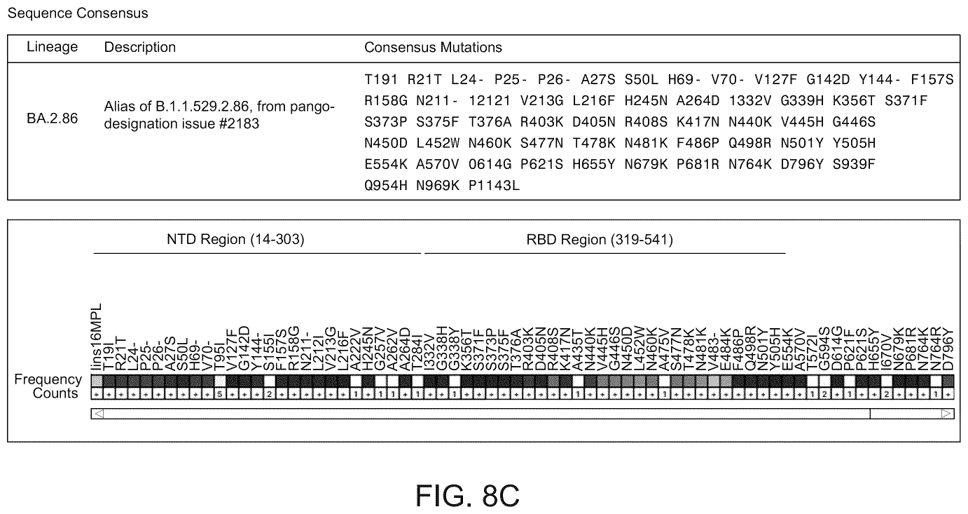

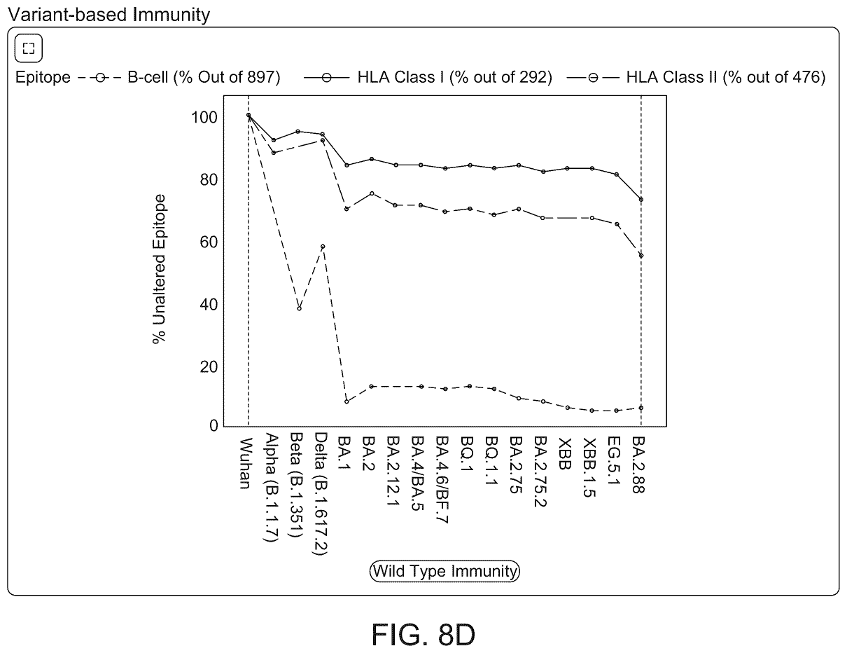

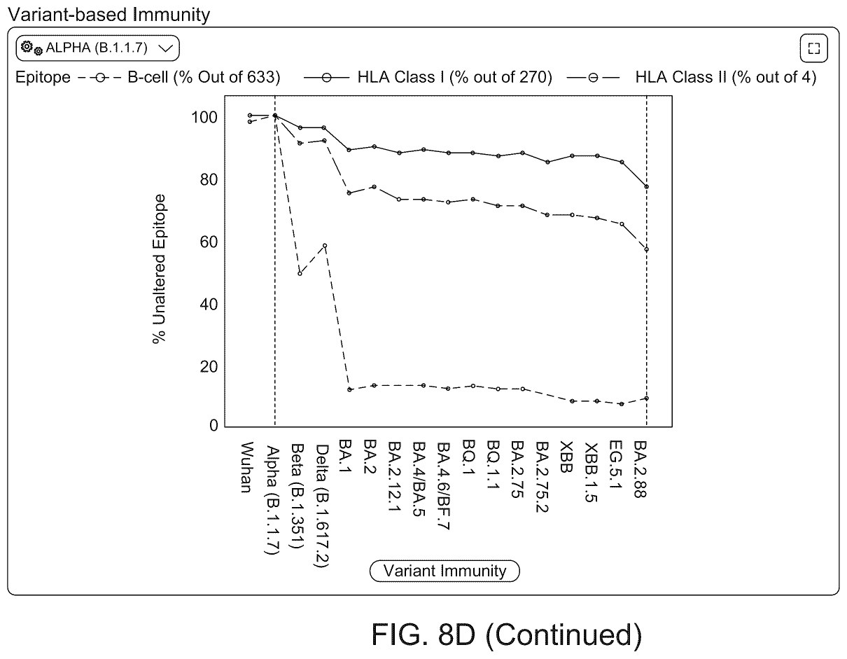

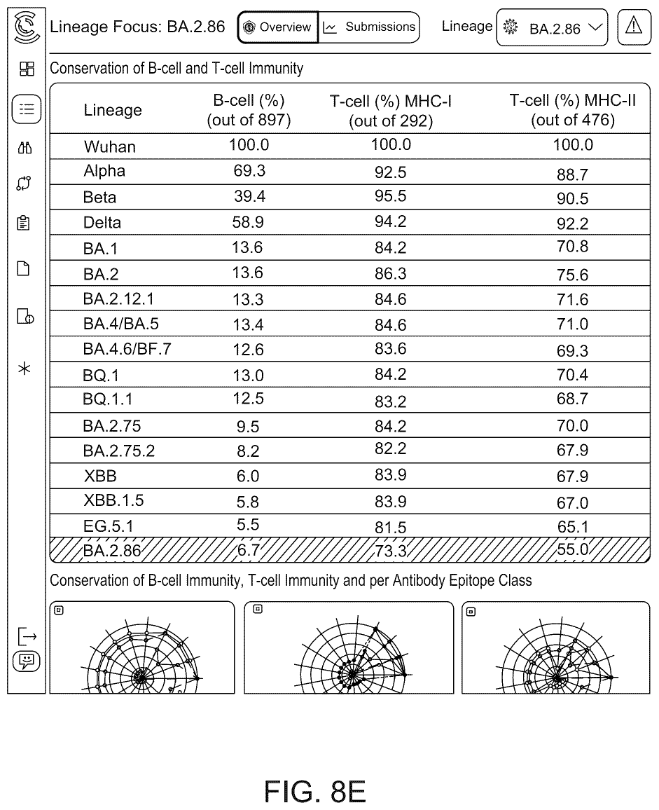

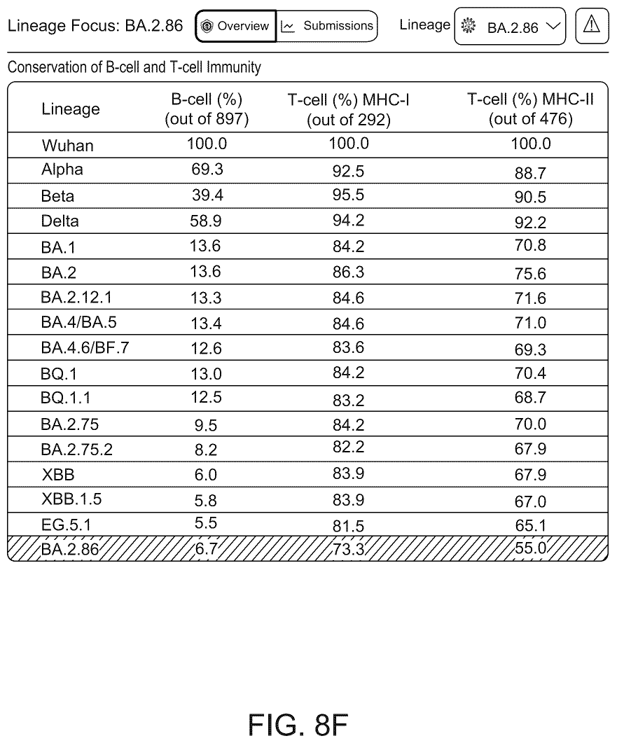

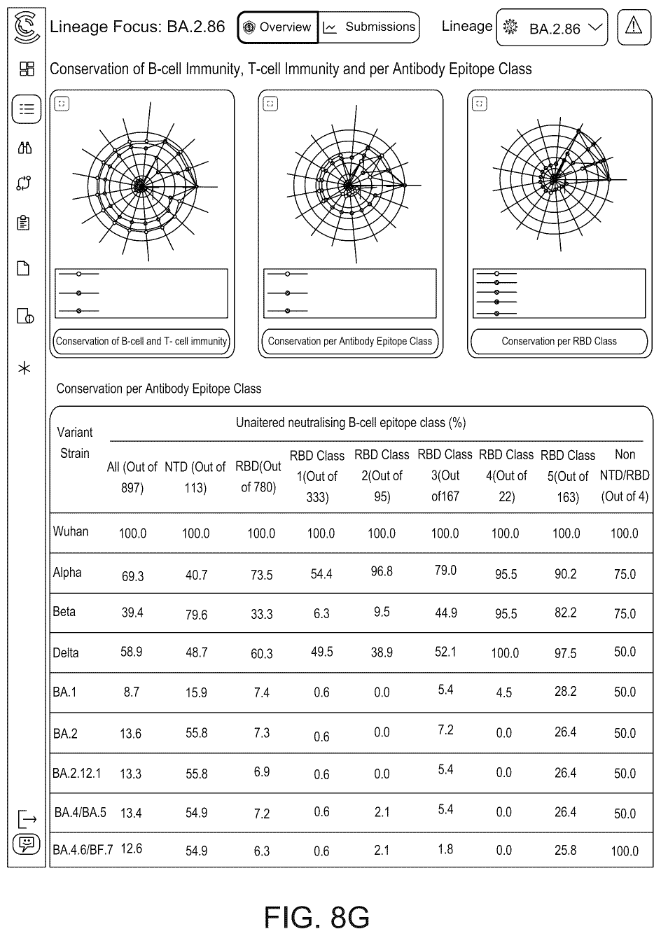

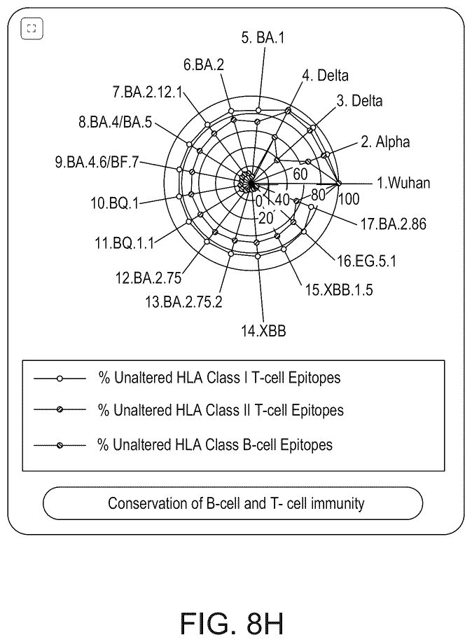

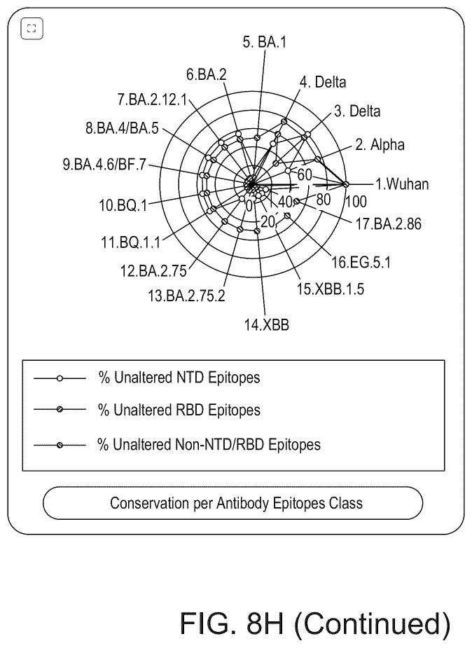

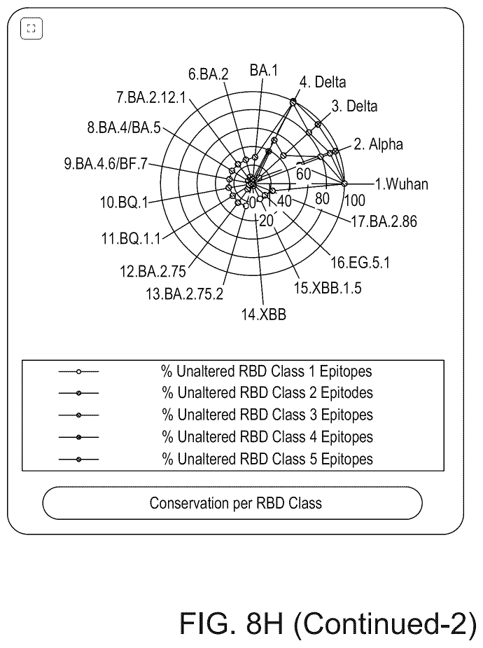

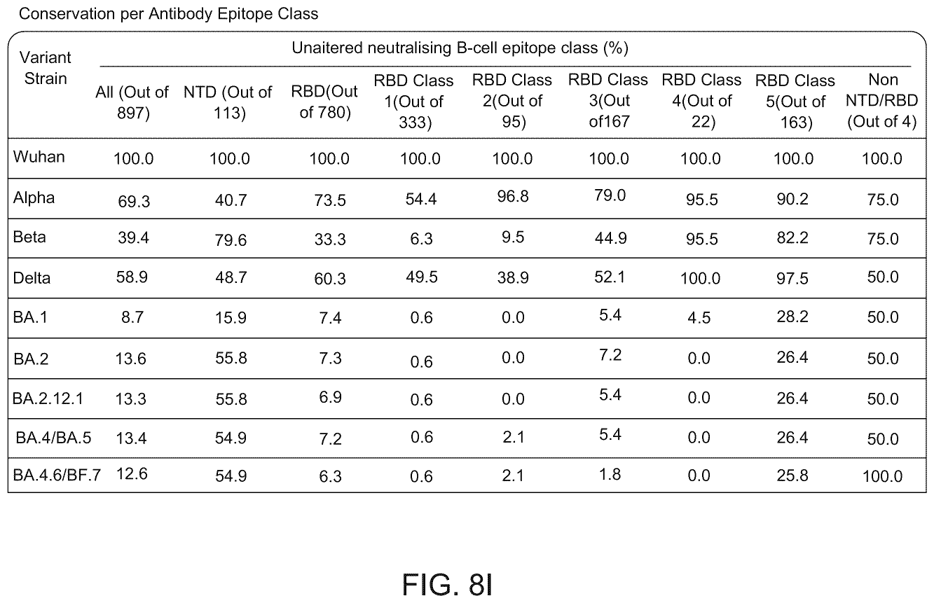

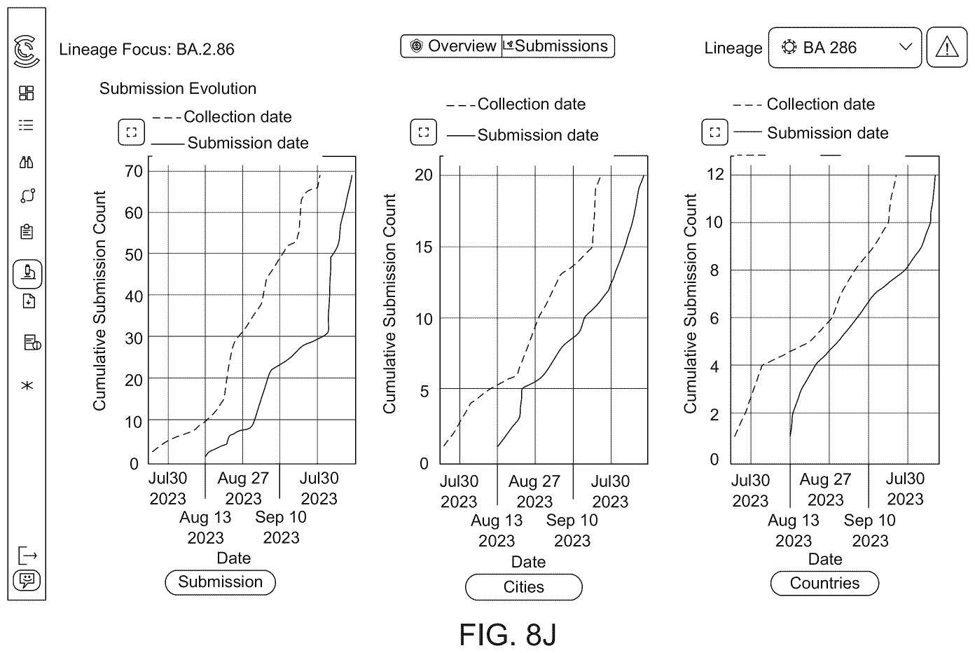

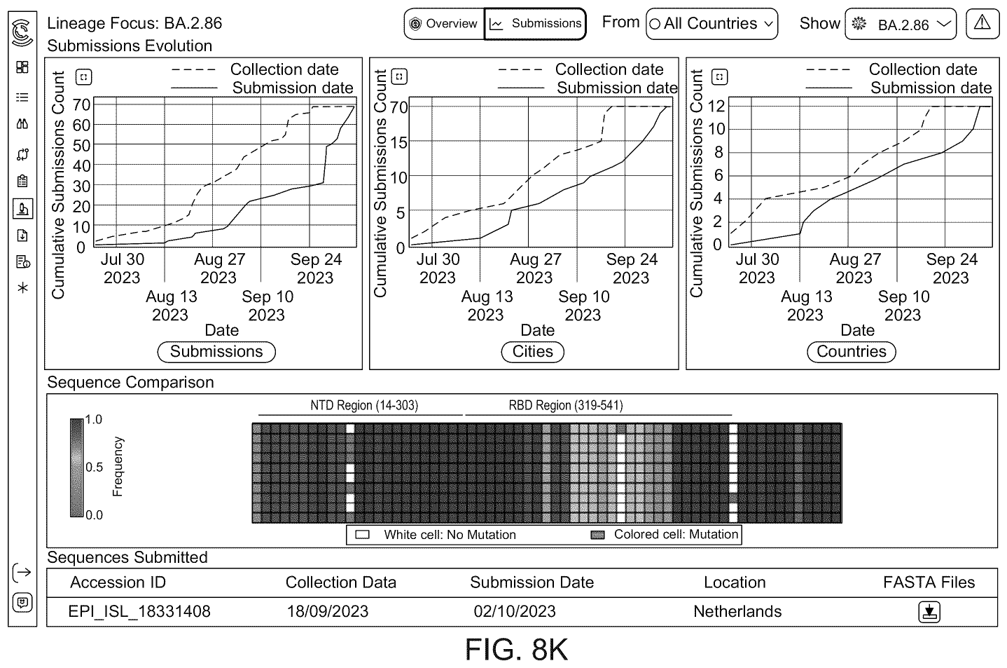

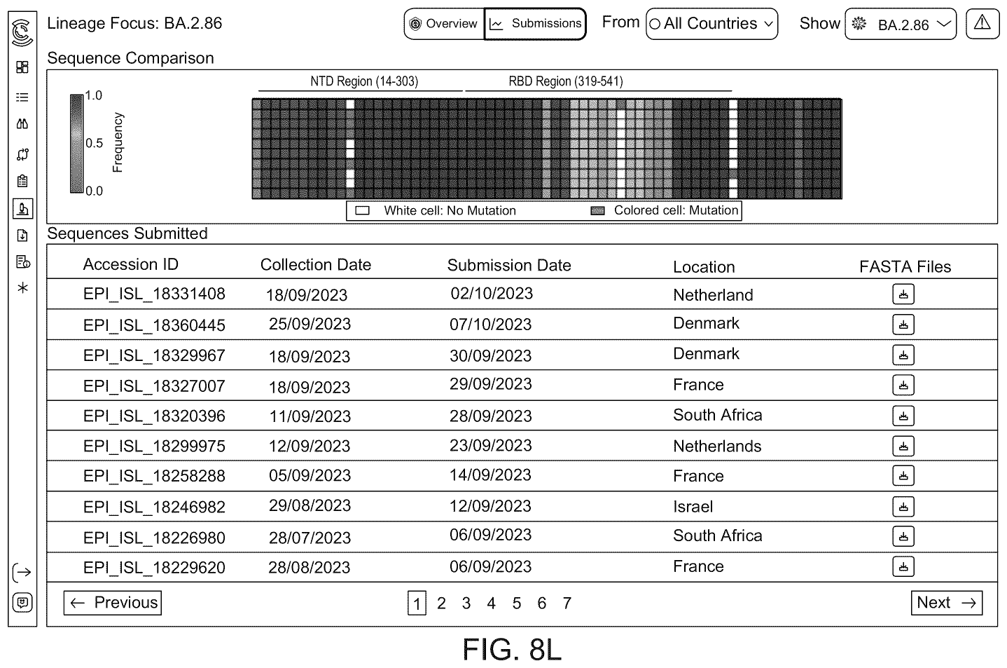

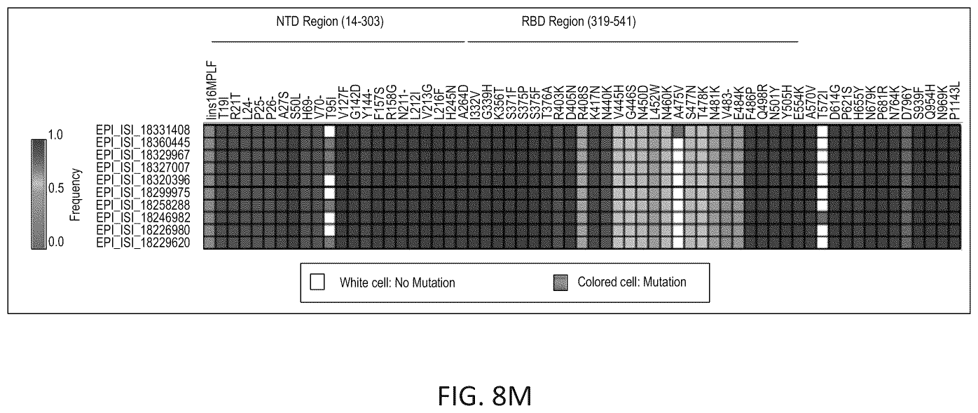

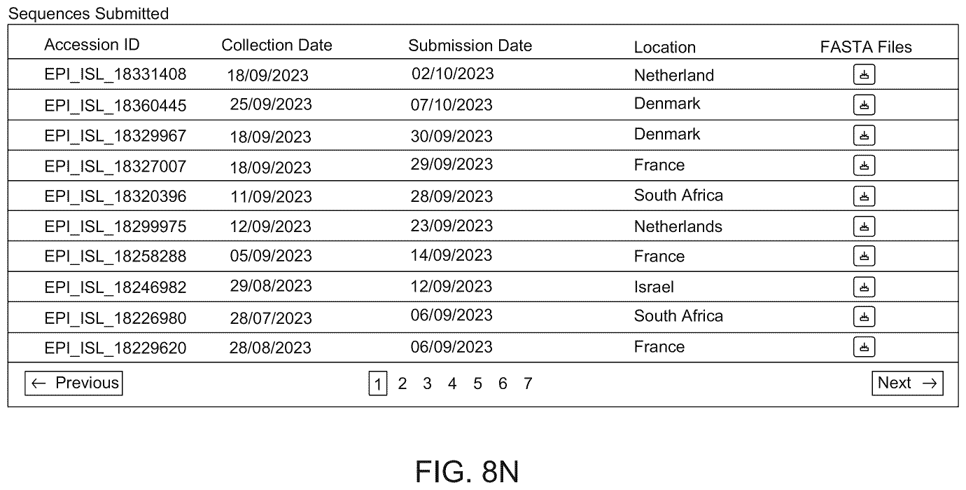

FIG. 8A shows a screenshot of an example lineage description page displaying a list of lineages, along with descriptions and consensus mutations for each. In certain embodiments, as used herein, consensus mutations refer to those mutations having occurred/been observed/present in greater than (e.g., or equal to) a threshold percentage of submitted sequences. The threshold percentage may be 50% or more, 60% or more, 75% or more, 80% or more, etc. In certain embodiments, a user may search for (e.g., via search box) and/or select a particular lineage as a focus lineage for further analysis.

FIGS. 8B-8N show screenshots of an example lineage focus page that a user may view and/or interact with to obtain useful insights on a particular viral lineage. For example, in the context of SARS-CoV-2, a user may select a particular lineage of interest, such as BA.2.86 for further review.

In certain embodiments, a lineage focus page comprises two tabs—an overview tab and a submissions tab. First tab—overview tab—conveys information on the selected lineage itself. Second tab—submissions tab—conveys information on submissions related to the lineage.

FIGS. 8B-8I show screenshots of an example lineage focus page when the overview tab is selected. Example lineage focus page shown in FIGS. 8B-8I may include several sections and/or graphical panels, such as one or more of the following:

(a) Sequence Consensus: In certain embodiments, a sequence consensus panel includes a visual display (e.g., text) conveying consensus mutations of a selected focus lineage.

(b) Consensus Color Heatmap: In certain embodiments, a color heatmap displays information about a frequency of mutations in submitted sequences related to the selected focus lineage.

FIG. 8C shows an expanded view of examples of sequence consensus panels and a consensus color heatmap.

(c) Variant Based Immunity Line Plots: In certain embodiments, line plots compare a percentage of conservation of B-cell Immunity and T-cell Immunity (MHC-I and MHC-II) among multiple lineages including the selected focus lineage. In certain embodiments, a user may interact with the GUI to select a comparison baseline. FIG. 8D shows an expanded view of variant based immunity line plots.

(d) Conservation of B cell and T cell Immunity Table: In certain embodiments, a table displays a percentage of conservation of B-cell immunity and T-cell immunity (MHC-I and MHC-II) for multiple lineages including the selected focus lineage. FIGS. 8E and 8F show an exemplary table of B cell and T cell immunity conservation.

(e) Conservation of B-cell Immunity, T-cell Immunity, and per Antibody Epitope Class Radar Plots: In certain embodiments, radar plots may be displayed which show a percentage of conservation for multiple lineages including the selected focus lineage. In certain embodiments, a first plot displays conservation of B-cell immunity and T-cell immunity (MHC-I and MHC-II). In certain embodiments, a second plot shows conservation per antibody epitope class. In certain embodiments, a third plot displays conservation by RDB Class. FIGS. 8G and 8H show exemplary radar plots graphing conservation of B-cell immunity, T-cell immunity, and per-antibody epitope class immunity.

(f) Conservation per Antibody Epitope Class table: In certain embodiments, a table displays the percentage of conservation per antibody class (NTD, RBD, RBD Classes 1 to 5 and non NTD/RBD) for multiple lineages including the selected focus lineage. FIG. 8I shows an example conservation per antibody epitope class table.

FIGS. 8J-8N show screenshots of an example lineage focus page when the submissions tab is selected. Example lineage focus page shown in FIGS. 8J-8N may include several sections and/or graphical panels, such as one or more of the following:

(a) Submissions Evolution Line Plots: In certain embodiments, one or more line-plots are displayed, conveying, e.g., as shown in FIG. 8J, information about the evolution of the number submissions, the number of cities where the lineage was submitted and the number of countries where the lineage was submitted. FIGS. 8J and 8K show example submission evolution line plots.

(b) Submissions Map: In certain embodiments, a heat map is displayed that conveys information about the number of submissions by country during a specific interval of time selected by the user.

(c) Sequence Comparison Color Heatmap: In certain embodiments, a color heat map displays information about the submitted sequences and their mutations as shown, for example, in FIGS. 8K-8M.

(d) Submitted Sequences Table: In certain embodiments, a lineage focus page includes a table conveying information about the submitted sequences, collection date, submission date, and location, as shown, for example, in FIGS. 8L and 8N.

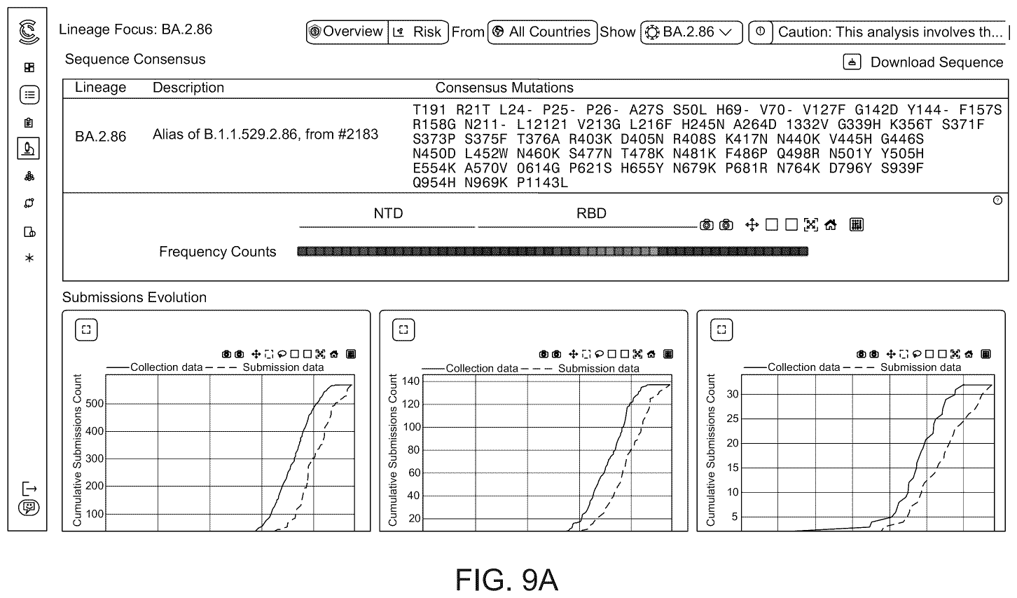

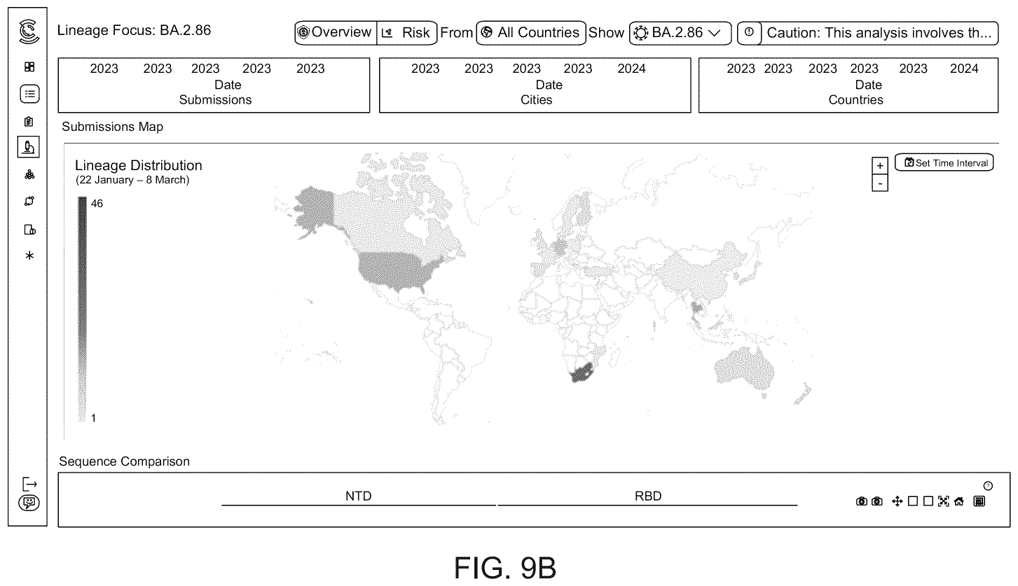

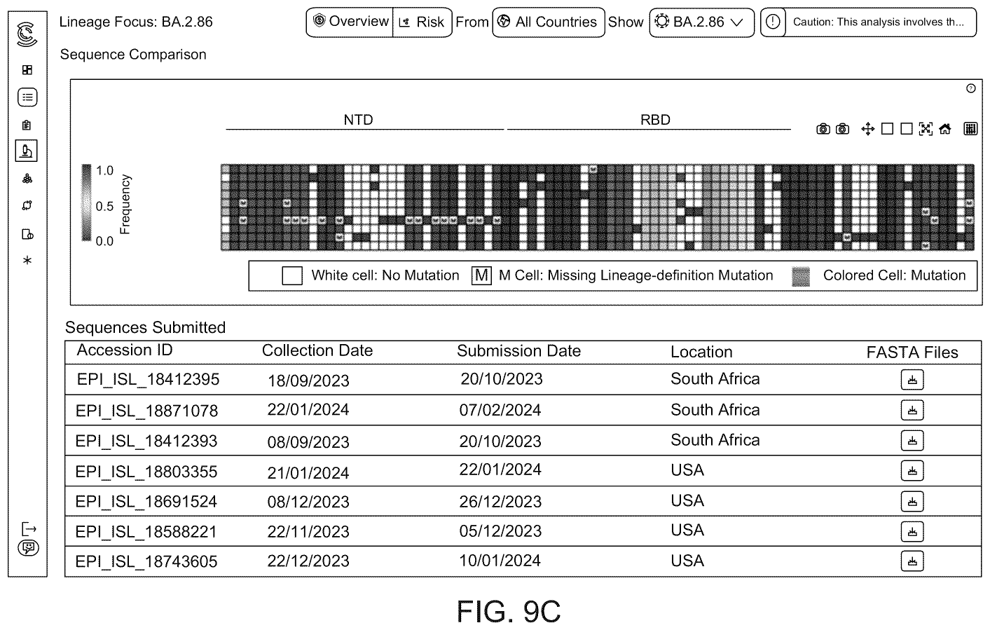

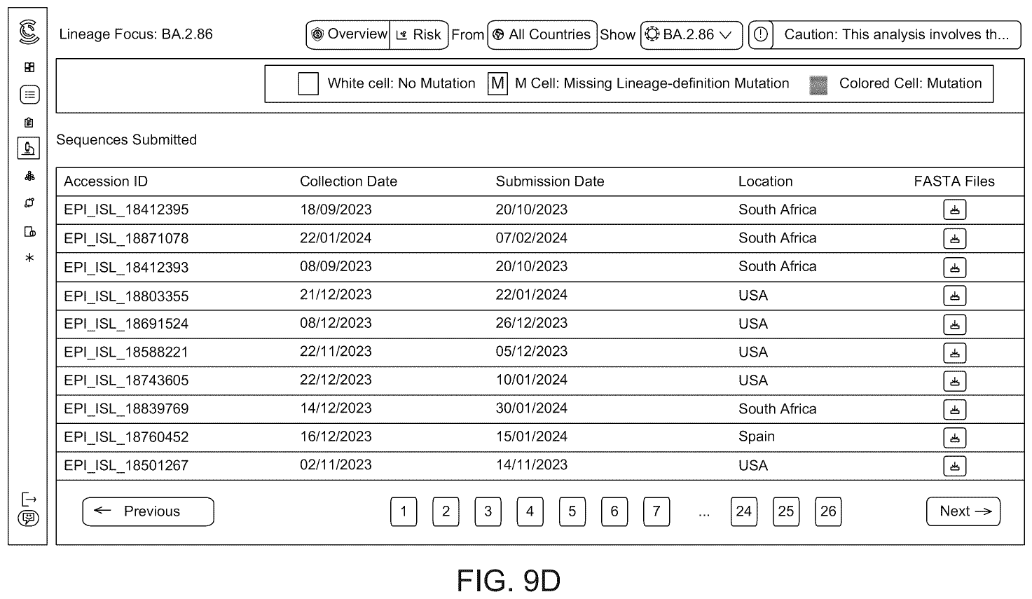

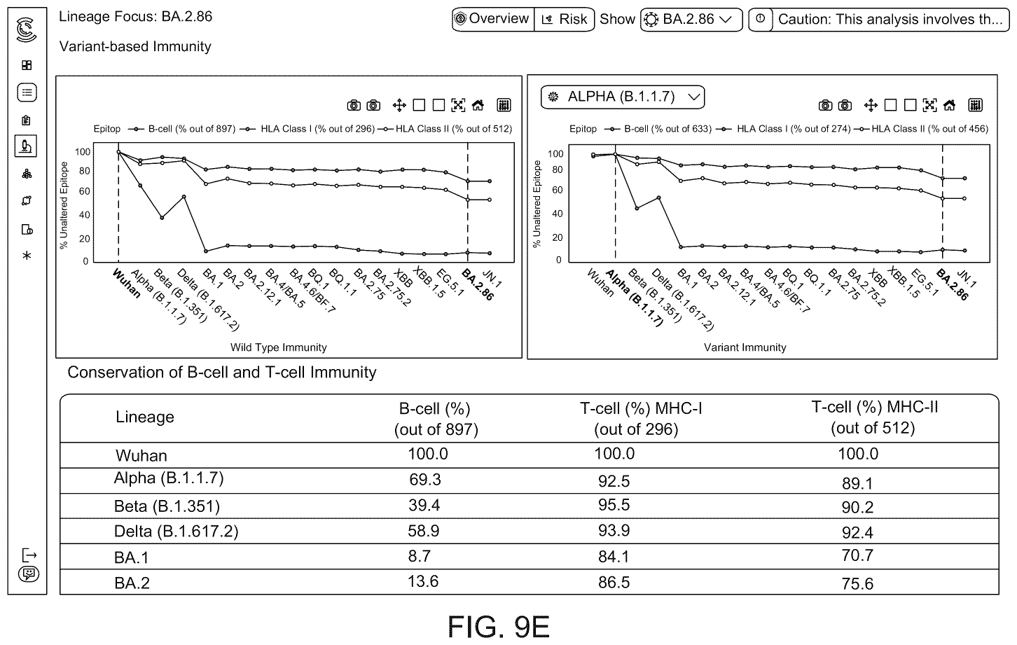

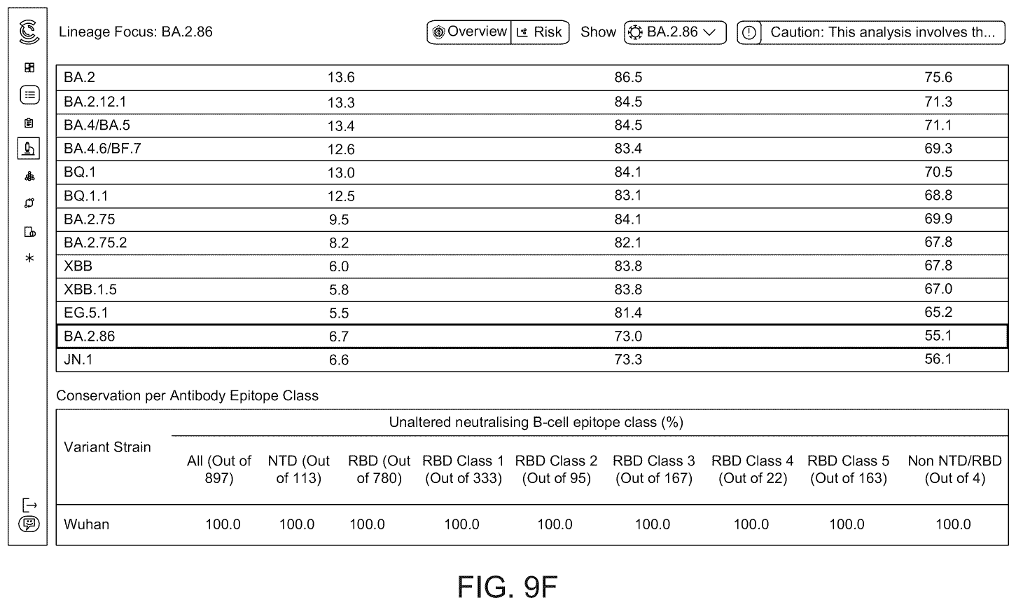

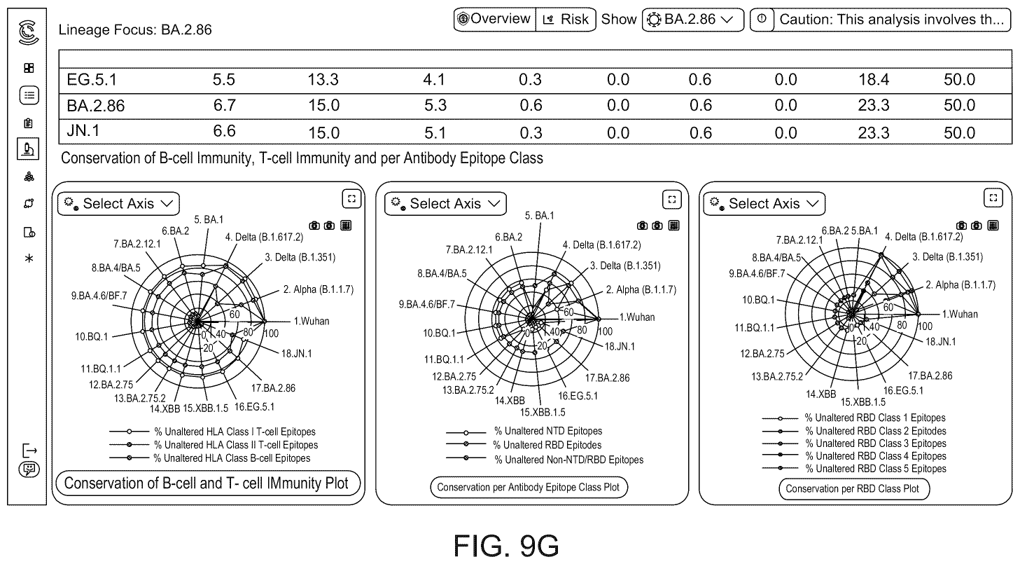

FIGS. 9A-9G are screenshots similar to those shown in FIGS. 8A-8N, providing additional or alternative embodiments of graphical user interfaces for inspecting lineages and relevant mutations.

FIG. 10 is a screenshot of a GUI providing weekly downloadable reports, according to an illustrative embodiment.

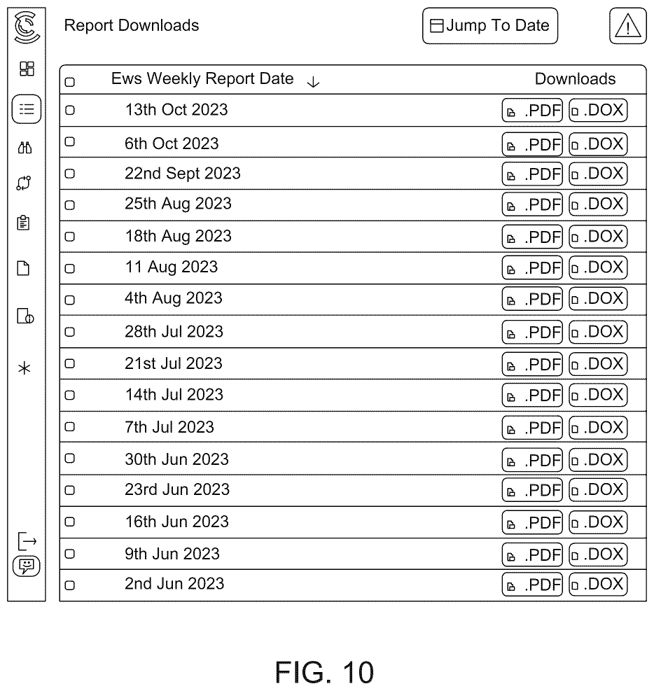

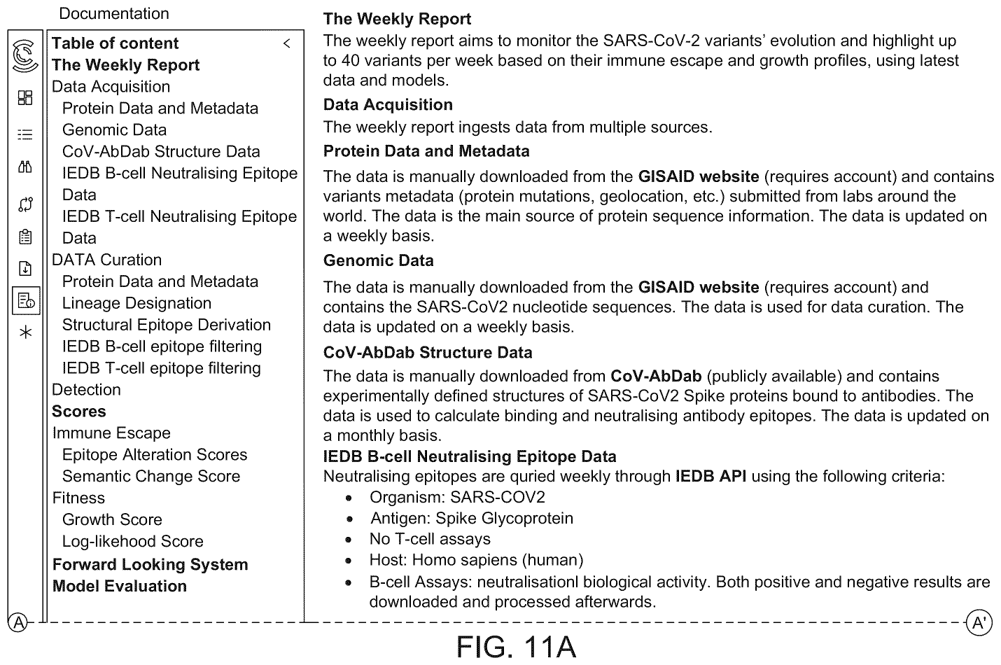

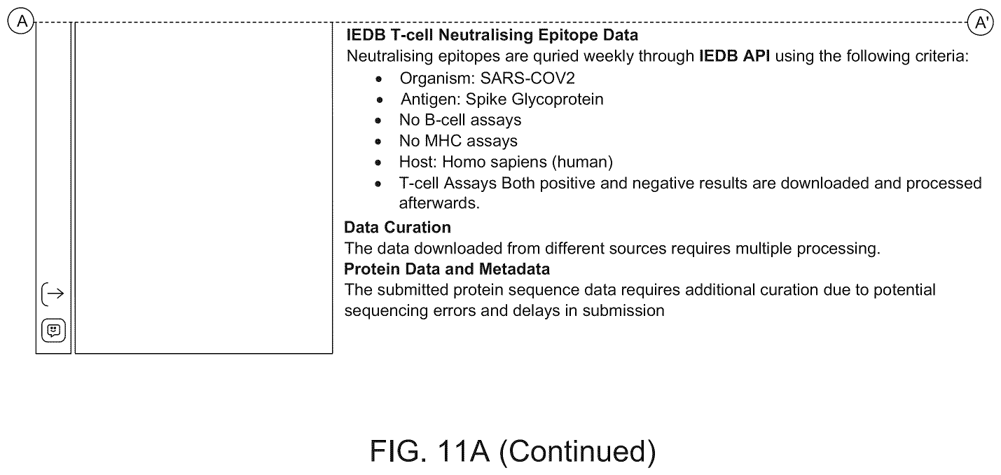

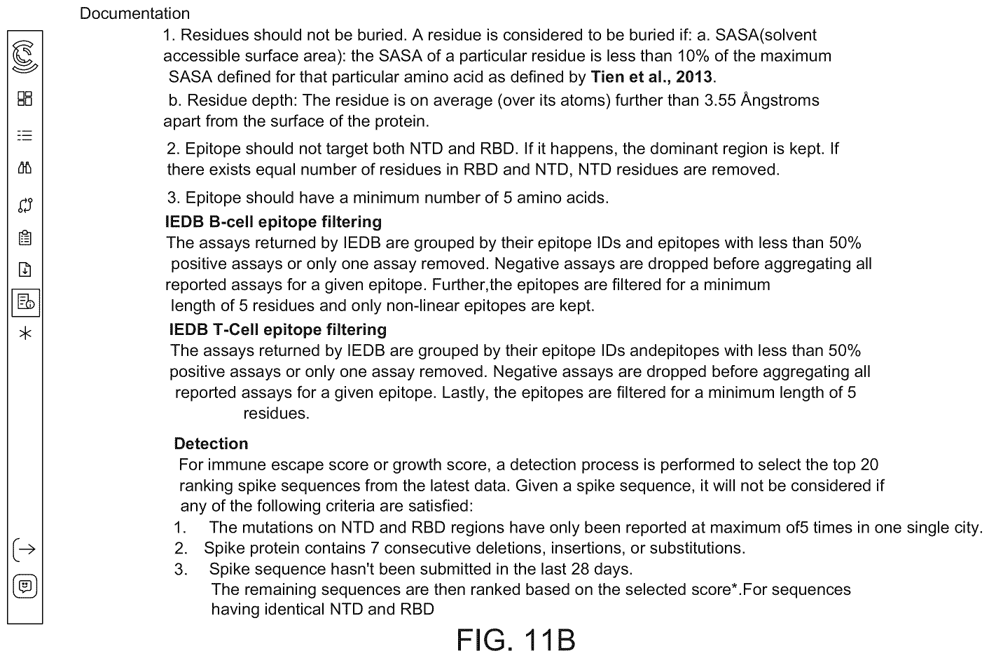

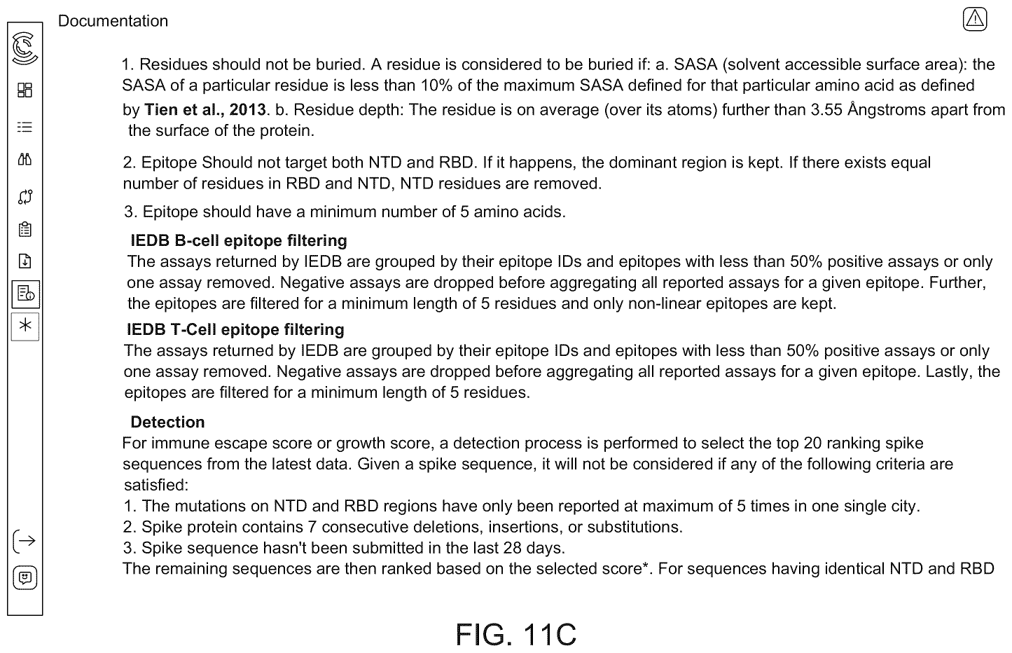

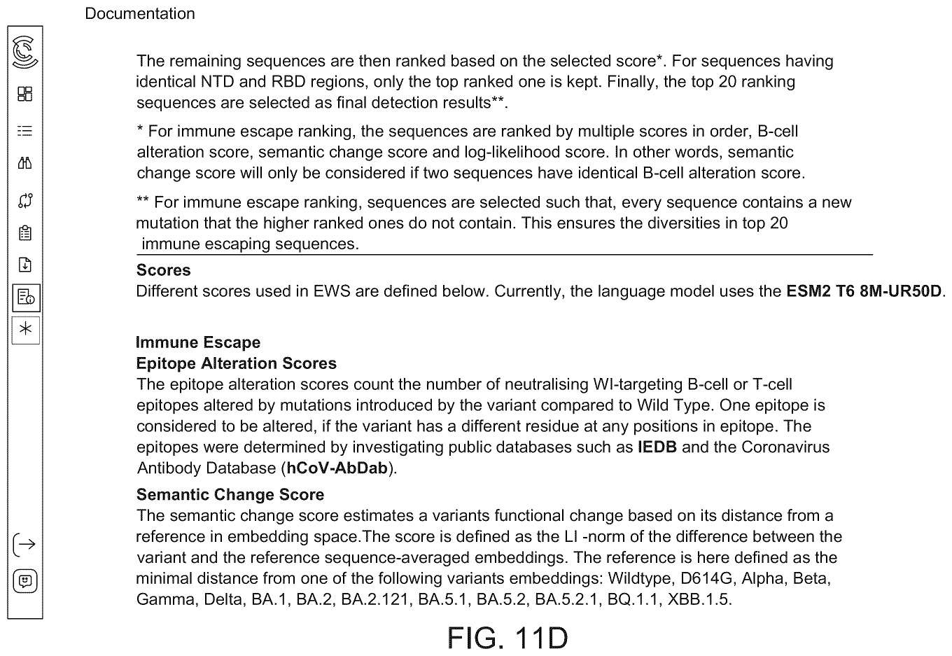

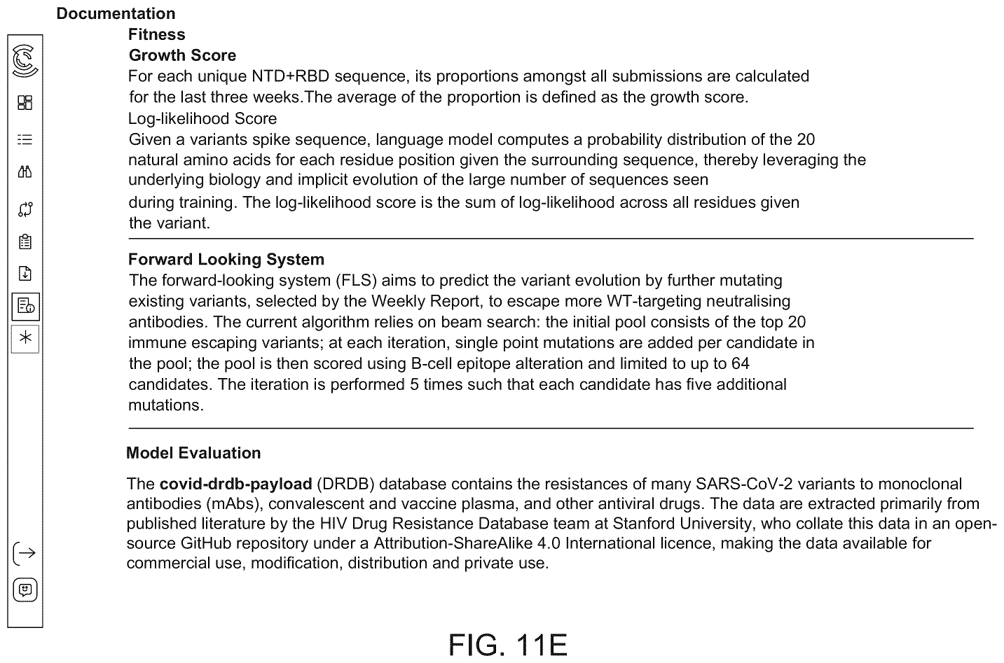

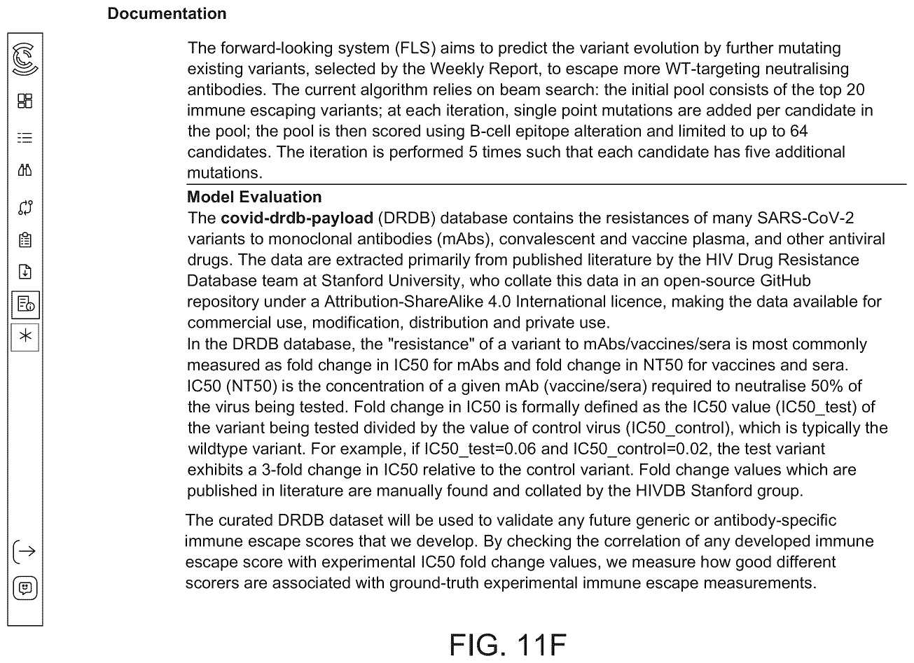

FIGS. 11A-11F are screenshots of portions of a GUI displaying documentation describing data sources and data analysis processes used by various technologies described herein, according to various illustrative embodiments.

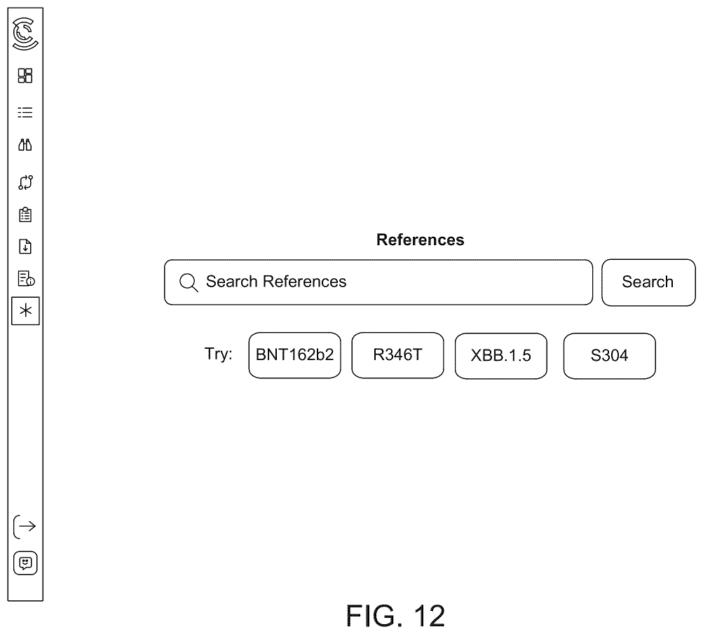

FIG. 12 is a screenshot of a GUI allowing a user to search for references pertaining to a particular viral variant, according to an illustrative embodiment.

In certain embodiments, data displayed/rendered via a decision support GUI may be received and/or accessed from a variety of sources, such as public datasets (e.g., NCBI, GISAID, IEDB, etc.), proprietary/private datasets, e.g., specific to a particular country, AI-based prediction system results as described herein, and local laboratory testing results.

Features, such as graphical panels, data display tables, and the like, of various GUI pages, e.g., as shown in FIGS. 4A-12 may be combined and/or substituted with each other in various combinations in various embodiments of DVRS systems according to the present disclosure. While exemplary screenshots shown in FIGS. 4A-12 show an example, functioning DVRS system developed for SARS-CoV-2, approaches described herein may likewise be applied and/or adapted to monitoring other infectious agents, such as influenza, norovirus, filoviruses, as well as bacteria and parasites (e.g., malaria), etc.

### D. CLUSTERING VARIANTS AND DISTRIBUTION FORECASTING

In certain embodiments, systems and methods of the present disclosure may be used to forecast distributions of viral variants at present and/or future time-points based on historical sequence data. Among other things, technologies of the present disclosure may include and leverage machine learning models to generate representations of submitted variant sequences and/or clustering methods to identify groups of similar and/or related variant sequences.

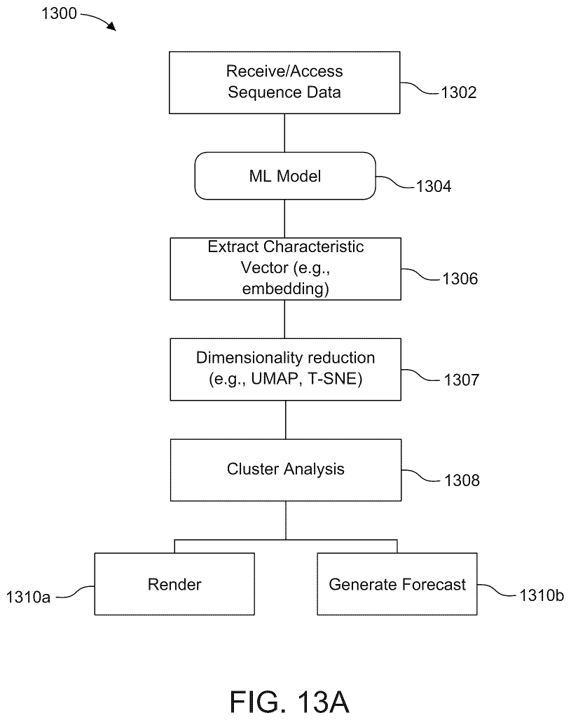

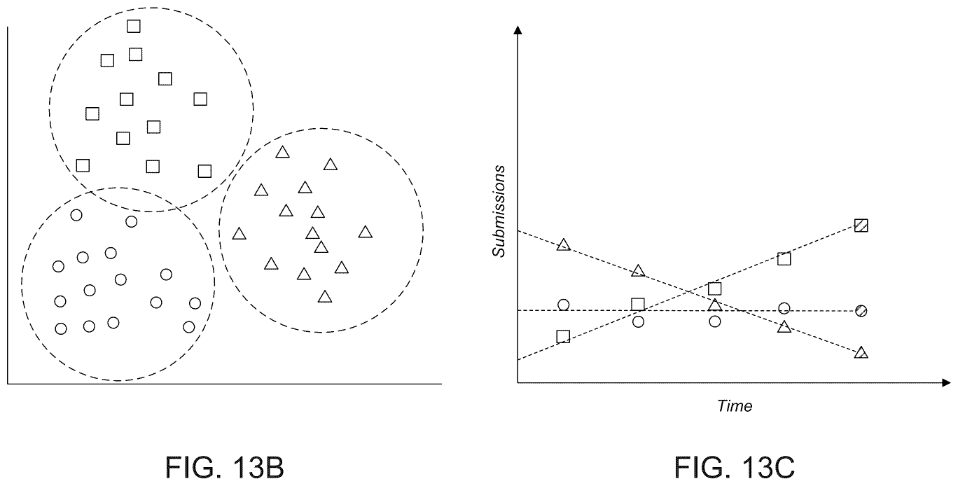

FIG. 13A shows an example process 1300 for generating variant distribution forecasts in accordance with certain embodiments described herein. As shown in FIG. 13A, in certain embodiments, variant distribution forecast techniques of the present disclosure utilize (e.g., received and/or accessed, or otherwise obtained) sequence data 1302 to track and/or generate predictions of distributions of various viral variants at current and/or future times. Sequence data may be, for example, historical sequence data, comprising sequences of polypeptides of viral variants that have been previously determined and submitted to one or more databases, such as, for example, local, regional, nationwide, worldwide database, run by, e.g., governmental organizations, private organizations, non-profits, public-private partnerships, and the like. For example, sequence data may be obtained from databases such as GISAID.

Sequence data may include sequences of circulating variants of various portions (e.g., polypeptides and/or portions thereof) of particular viral pathogen, such as SARS-CoV-2, influenza, RSV, human monkypox (hMPXV), and the like. Sequence data may include sequences of particular polypeptides and/or portions thereof, such as SARS-CoV-2 spike protein sequences (e.g., a full spike protein sequence), as well as, additionally or alternatively, sub-units or portions thereof, such as a receptor binding domain (RBD) and/or an N-terminal domain (NTD) thereof. Sequence data may include sequences generated by various organizations (e.g., labs, hospitals, etc.) over various regions of observed viral pathogens, obtained from samples from e.g., individuals as they are tested via regular surveillance and/or during visits to medical facilities, e.g., when seeking treatment. Sequences may be submitted over time, e.g., on an ongoing, basis, as they are generated. Sequence data may, accordingly, be updated on a continuous or near-continuous basis, and/or at regular intervals of time, such as daily, weekly, bi-weekly, monthly, etc.

In certain embodiments, for example as shown in FIG. 13A, sequence data is provided, as input, to a machine learning model 1304. In particular, in certain embodiments, each of at least a portion of variant polypeptide sequences (within sequence data 1302) are provided, as input to a machine learning model. For each sequence provided as input to machine learning model 1304, machine learning model 1304 may be used to generate a characteristic vector. A characteristic vector may, for example, be generated based on an internal representation of machine learning model 1304, also referred to as an embedding, which can be extracted 1306. An embedding may be, for example, extracted as output from an internal (e.g., hidden) layer of a machine learning model 1304 and may represent an input sequence as a high dimensional matrix or vector (e.g., a numerical matrix or vector). For example, in certain embodiments, machine learning models, such as certain language models described in further detail herein, may receive an amino acid sequence as input and generate, an internal (e.g., embedding) representation. In certain embodiments, an embedding is or comprises a vector (e.g., zi), e.g., of length D, where D is a dimensionality of the vector, for each amino acid position in a sequence, such that an initial embedding corresponding directly to an internal representation output by a particular layer (e.g., a final embedding layer of a recurrent neural network, e.g., a feature map from a final transformer layer of a transformer-based model) may be a matrix of size n×D [e.g., or (n+1)×D], where n is a length of an input amino acid sequence and D is a number of dimensions [e.g., in certain embodiments, an additional, class token may be used and appended to a network, such that a matrix is of a size (n+1)×D], based on the particular machine learning model used. In certain embodiments, a characteristic vector may then be determined as a mean over at least a portion of sequence positions {e.g., all sequence positions, [e.g., but excluding a first (e.g., class token) position, not representing or corresponding to an amino acid in the sequence]} to, for example determine a characteristic vector z, for an amino acid sequence. Further details of embedding vectors and approaches for determining characteristic vectors based thereon are provided in in PCT publications WO 2022/235847 and WO 2022/235853.

In this manner, in certain embodiments, machine learning model 1304 may be used to generate, for each of a plurality of viral variant polypeptide sequences, a corresponding characteristic vector (e.g., based on an embedding of machine learning model 1304). As illustrated in FIG. 13B, this approach can be used to associate each variant sequence with a location in a higher dimensional characteristic vector/embedding space. FIG. 13B illustrates a 2D space, and may be generated, for example, via a dimensionality reduction technique (e.g., T-SNE), but it should be understood that characteristic vectors representing viral variant sequences may be vectors of any length (e.g., 10, 100, 1,000, 10,000, etc.).

Machine learning models used to generate characteristic vectors may utilize and implement a variety of machine learning techniques. For example, machine learning model 1304 may be a deep learning model (e.g., an artificial neural network with one or more, e.g., a plurality of, hidden layers), such as a language or large language model (LLM). In certain embodiments, machine learning model 1304 is or comprises one or more recurrent models, such long short-term memories (LSTMs), implemented alone or in combination, e.g., as in a bi-directional LSTM (bi-LSTM). In certain embodiments, machine learning model 1304 may be or comprise one or more transformer models. Examples of machine learning models include, without limitation, evolutionary scale models (ESM), bidirectional encoder representations from transformers (BERT), and the like.

Machine learning model 1304 may be trained, for example on protein sequence data available from public and/or private repositories, such as UniRef (see, e.g., Suzek et al. “UniRef: comprehensive and non-redundant UniProt reference clusters” 2007), the Virus Pathogen Resource (ViPR) database by the National Centre for Biotechnology Information, and the like. Training may be accomplished by providing a machine learning model with input sequences that are incomplete and/or partially masked and tasking them to predict the omitted and/or masked data. In this manner, machine learning models may be trained in an unsupervised fashion. Additional details of machine learning models and approaches for training them, such as particular techniques for training recurrent and/or transformer models, may be found, for example, in PCT publications WO 2022/235847 and WO 2022/235853.

In certain embodiments, while, during training, machine learning models generate, as output predictions of amino acid side chain types, such as predicted likelihoods of each side chain type, as described herein, internal representations (e.g., and not the amino acid likelihood output), that is, characteristic vectors are determined (e.g., based on embeddings generated internally) during inference, and used to generate forecasts of variant distributions.

Extracted characteristic vectors (e.g., based on embeddings) may be used directly, or, in certain embodiments, may be processed via a dimensionality reduction technique, such as t-distributed stochastic neighbor embedding (t-SNE), uniform manifold approximation and projection for dimension reduction (UMAP), and the like, to produce a reduced (dimension) characteristic vector 1307. For example, in certain embodiments, machine learning models such as ESM-1 may be used to generate large characteristic vectors, having a dimension (e.g., size) of over 100 (e.g., vectors of size 100 or more, 200 or more, 400 or more, about 480 or more, 500 or more, 1,000 or more, etc.). Large characteristic vectors may, in certain embodiments, be challenging to process directly by, for example, certain clustering approaches (e.g., without wishing to be bound to any theory, this may be considered a “curse of dimensionality”). In certain embodiments, dimensionality reduction techniques may, for example, be used to project characteristic vectors to a lower dimensional space, thereby reducing dimensionality (e.g., creating a reduced dimensionality version of the characteristic vectors). In certain embodiments, these reduced dimensionality vectors may have a size of about 100 (features) or less (e.g., 90 or less, e.g., 75 or less, e.g., about 60 or less, e.g., 50 or less). In certain embodiments, various approaches may be used to balance preservation of relevant structure in high-dimensional characteristic vectors while mapping them to a lower dimensional space to obtain reduced dimensionality vectors of a size more readily used by various clustering techniques. Various approaches for generating reduced dimensionality versions of characteristic vectors in this manner include, without limitation, t-SNE, UMAP, and the like.

Turning again to FIGS. 13A and 13B, FIG. 13B shows an illustrative, hypothetical, 2D projection of a plurality of characteristic vectors, and/or reduced dimensionality versions thereof, of variant sequences, e.g., a characteristic vector or embedding space representation and/or reduced dimensionality mapping thereof (e.g., obtained via a dimensionality reduction technique, such as t-SNE, UMAP, etc.). Similar variants are located nearby (e.g., in characteristic vector or embedding space), and accordingly, can be grouped as belonging to various clusters via clustering analysis 1408. Subdividing variant sequences into one or more clusters can be accomplished using a variety of clustering techniques, such as k-means clustering. As illustrated in FIG. 13B, variant sequences with characteristic vector representations, and/or lower dimensionality mappings thereof, that lie close to each other (in characteristic vector/embedding space and/or lower dimensionality mappings thereof) are identified as belonging to one cluster, while other variant sequences are assigned to other clusters. Additional details of clustering approaches and their use in connection with variants of viral polypeptides, may be found, for example, in PCT publications WO 2022/235847 and WO 2022/235853.

In certain embodiments, a size of each cluster, e.g., in terms of a number of submissions of variant sequences within the cluster, can be determined, and tracked over time, e.g., as shown in FIG. 13C. In certain embodiments, sizes of clusters can be used as input to various approaches for generating predictions at (e.g., recent and/or current) times with limited and/or incomplete data (e.g., a low number of submissions) and/or future times (e.g., with no submissions) that forecast how sizes of particular clusters will evolve over time. This approach can be used, for example, to project absolute and/or relative distribution of viral variant across determined clusters, at time points when insufficient or no submission data exists. Example approaches include, without limitation, time-series regression, such as autoregressive models (e.g., autoregressive moving average (ARIMA) models), machine learning models, such as support vector machines (SVMs), artificial neural networks (ANNs), and the like.

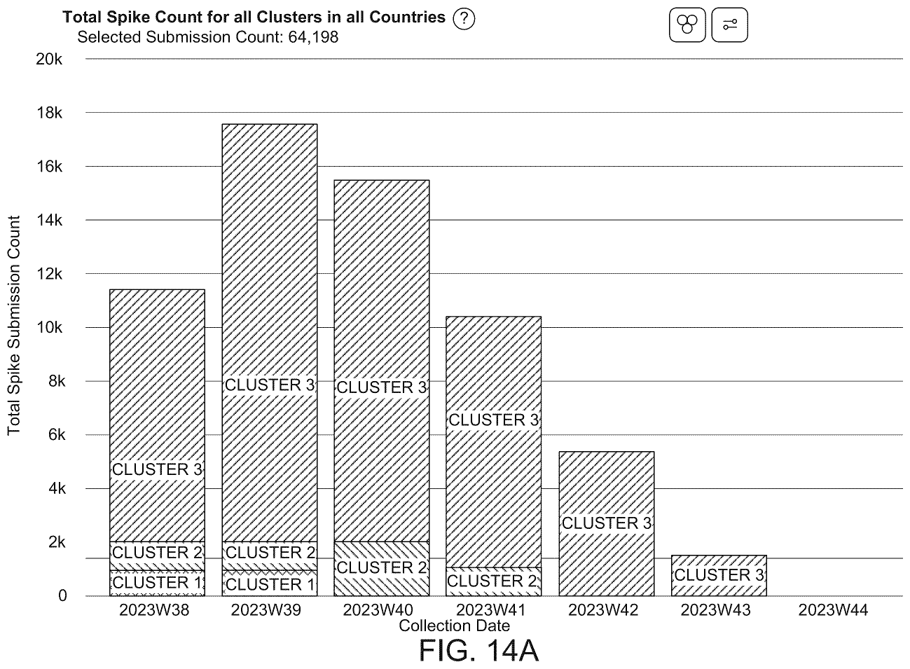

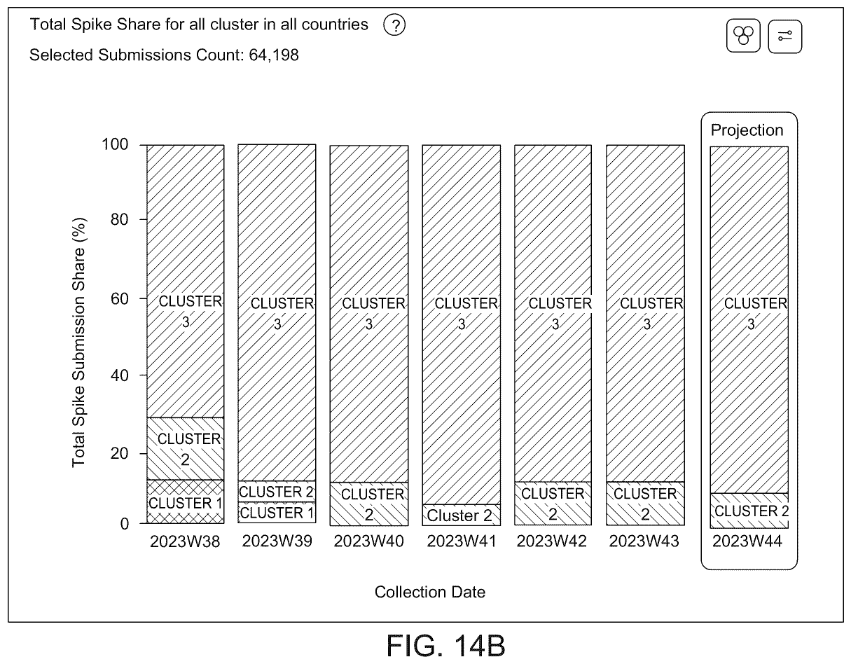

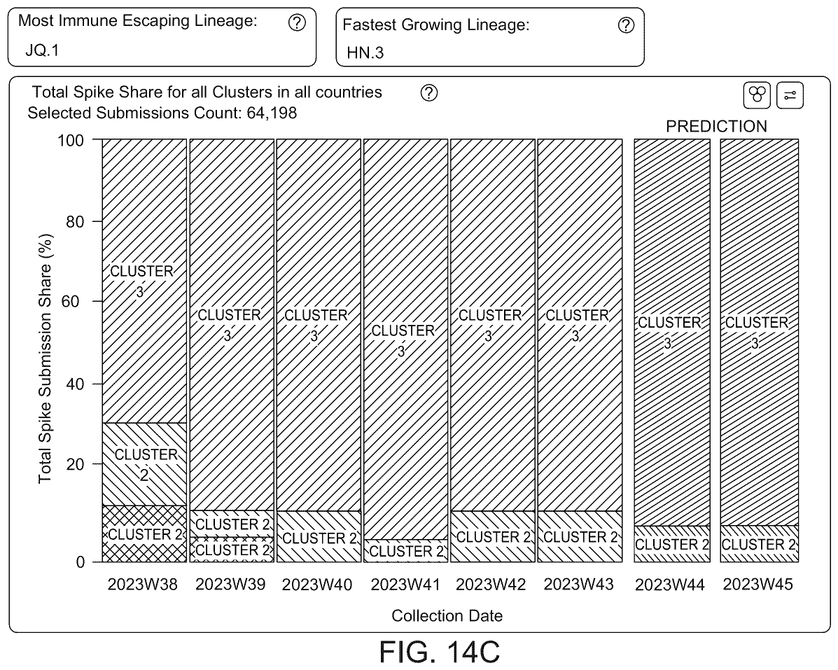

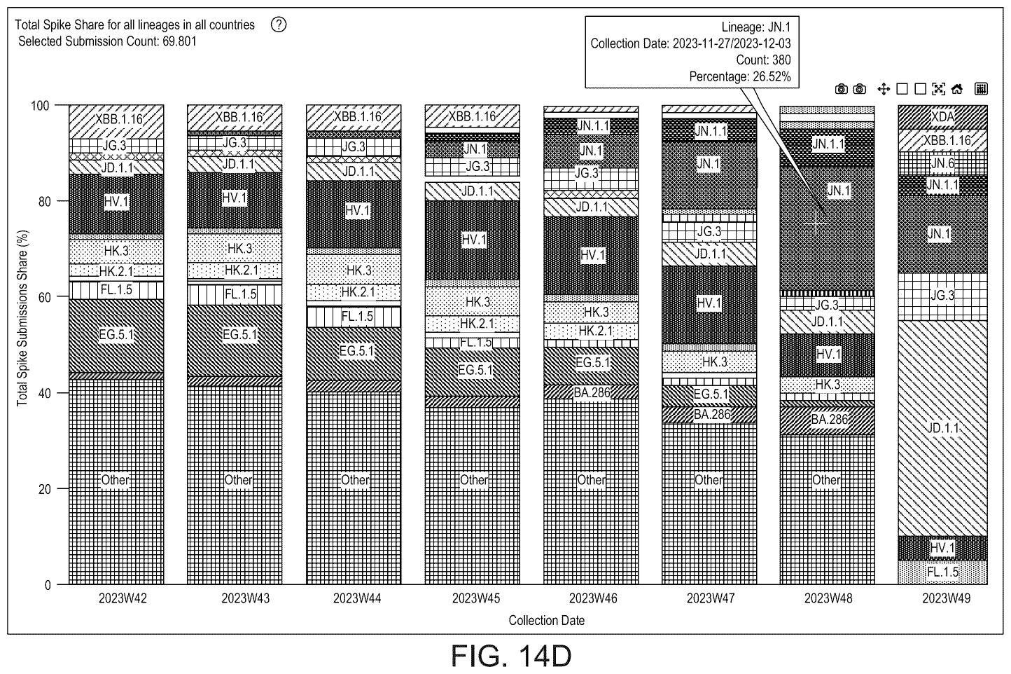

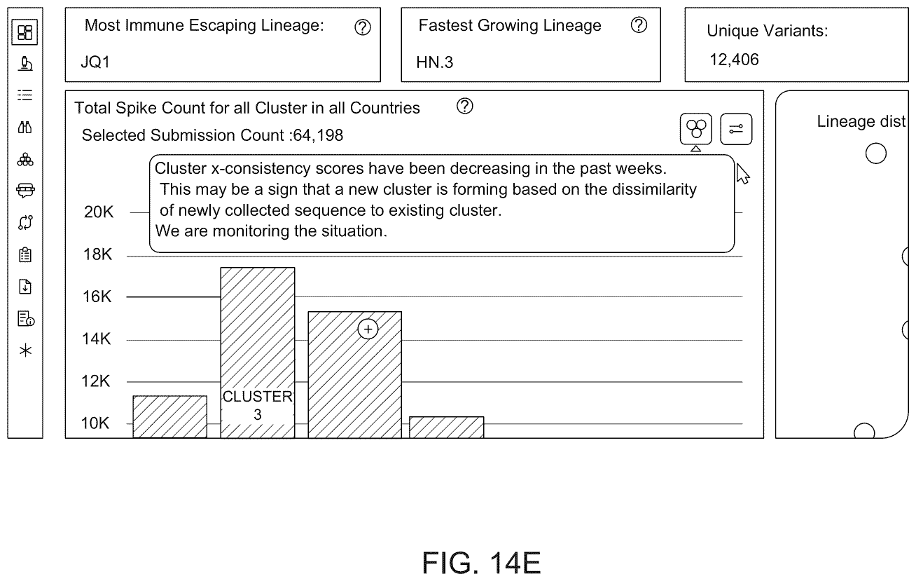

Moreover, in certain embodiments, and without wishing to be bound to any particular theory, clustering approaches as described herein facilitate analysis and projecting evolution of viral variants over time. In particular, as can be seen via a comparison between FIGS. 14B and 14D, clustering can be used to reduce granularity in data, making data analysis and projections less sensitive to noise, outliers, limited data, and the like. Characteristic vectors determined using machine learning model-based analysis of variant sequences relate to and may be predictive of underlying biological similarity between variants, and, accordingly, distances between them indicative of, e.g., immune escape. Accordingly, among other things, by performing clustering on characteristic vectors generated using machine learning models (e.g., based on machine learning model-generated embeddings), identified clusters and their evolution over time can relate to and provide informative information pertaining to biological, and, in turn, public health, implications of evolution of circulating variants. In this manner, among other things, cluster analysis and tracking techniques described herein can facilitate decision making by medical practitioners, public health officials, and the like.

As shown, for example, in FIGS. 14A-14E, in certain embodiments, cluster sizes and/or distributions of variant sequences across clusters can be rendered for graphical display 1310a. As shown in FIG. 14A, in certain embodiments, a visualization (e.g., a graph, chart, etc.) showing variations in an absolute size of each of one or more identified clusters can be rendered and displayed to a user, for example, as part of a web-portal GUI as described herein. For example, FIG. 14A shows variation in total size (e.g., measured by total spike submission count within a particular cluster) for three clusters on a weekly basis.

Turning to FIG. 14B, in certain embodiments, relative cluster sizes can be computed and rendered for display. In certain embodiments, a projection, as described herein may also be computed and included in a graphical chart rendered for display. Projections may be computed and/or displayed graphically at one or more time points, which, as described herein, may be recent and/or current time points, where limited data (e.g., low number of submissions) exists and/or future times. For example, FIGS. 14B and 14C show example projections/predictions for one and two weeks, respectively.

In certain embodiments, by assigning viral variant submissions according to clusters, as opposed to, for example, lineages, can provide a more readily understood and informative view into how circulating variants are evolving over time. This approach can be particularly valuable where limited data exists, as can be seen by comparing FIG. 14D (e.g., showing granular allocation of submissions according to lineages) with FIGS. 14B and 14C (e.g., showing results of clustering approaches as described herein).

In certain embodiments, one or more cluster consistency scores may be determined. Examples of consistence scores include measures of relatedness of embedding vectors to the cluster they were assigned to (e.g., silhouette score, Calinski-Harabasz Index, Davies-Bouldin index, etc.), cluster size, compactness, distance from other clusters and combinations thereof. Consistency scores and/or graphical icons, flags, and the like, based thereon, may be displayed within a GUI, for example, to alert a user of changes in, for example, a number and/or nature of clusters. For example, FIG. 14D shows an alert and popup message displayed within a GUI, alerting a user of a decrease in a particular clustering metric (e.g., an x-consistency score, where x may be one or more particular clusters, e.g., of a determined set of clusters) and a potential biological basis for the change.

### GUI Tools for Cluster Analytics

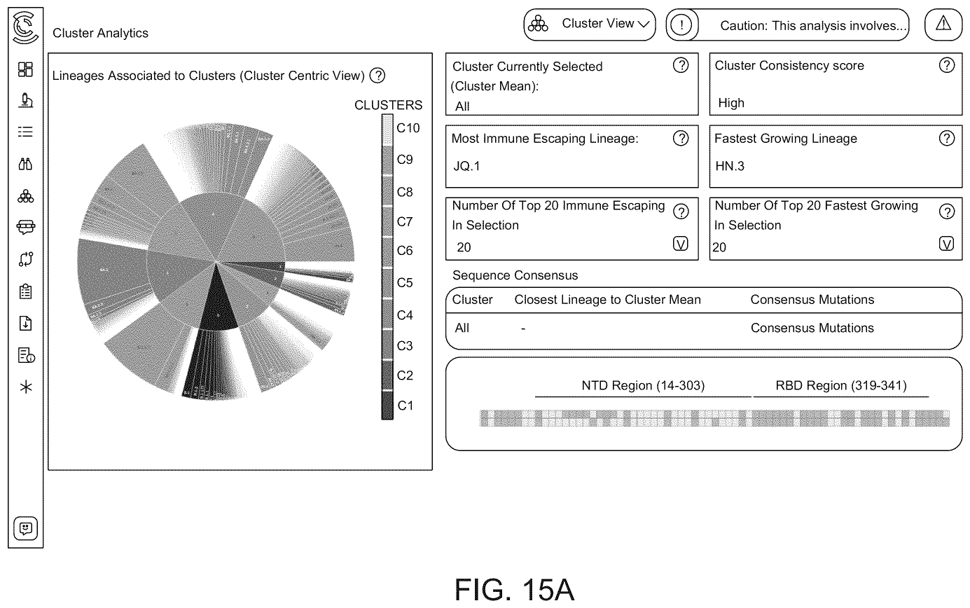

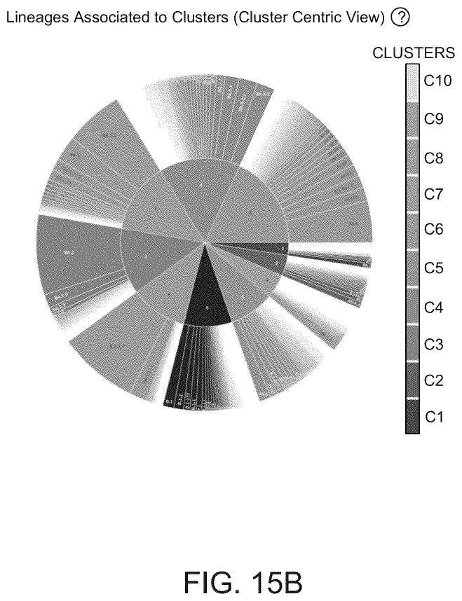

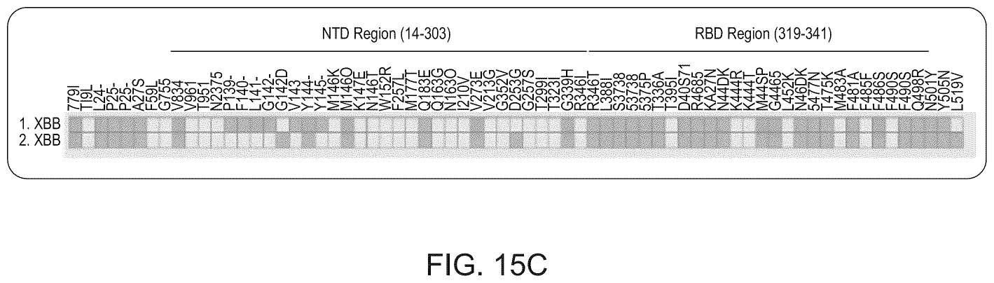

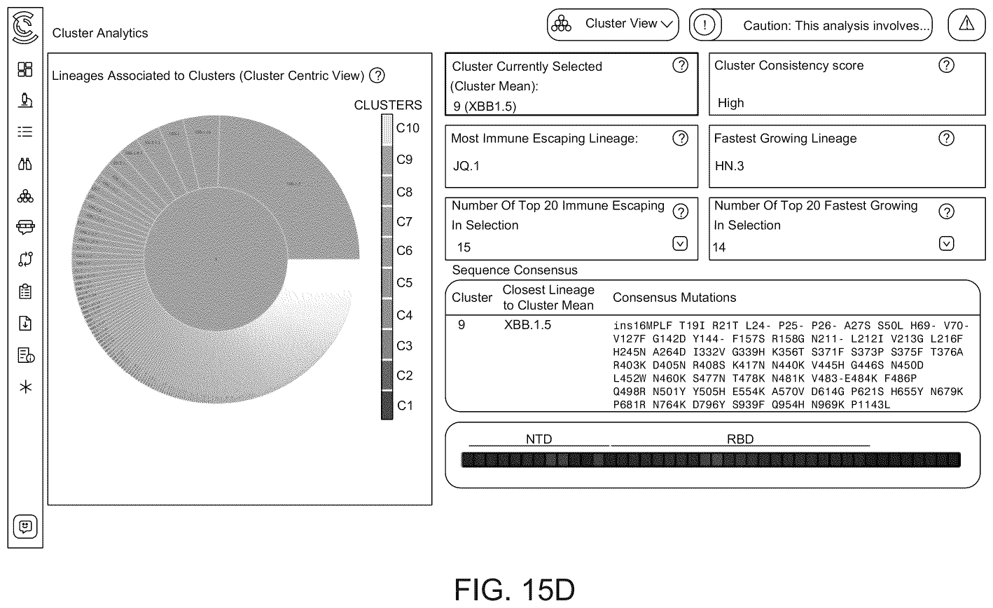

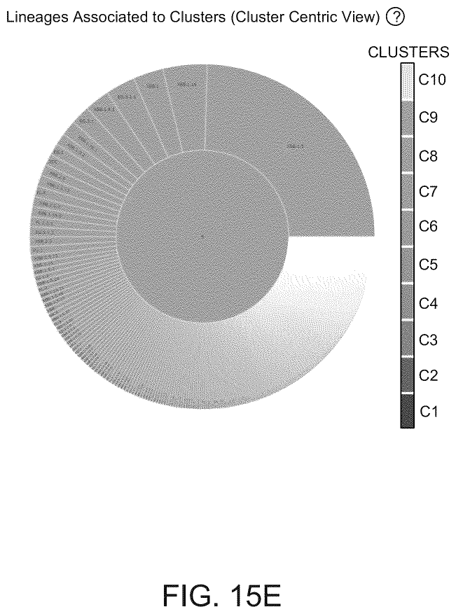

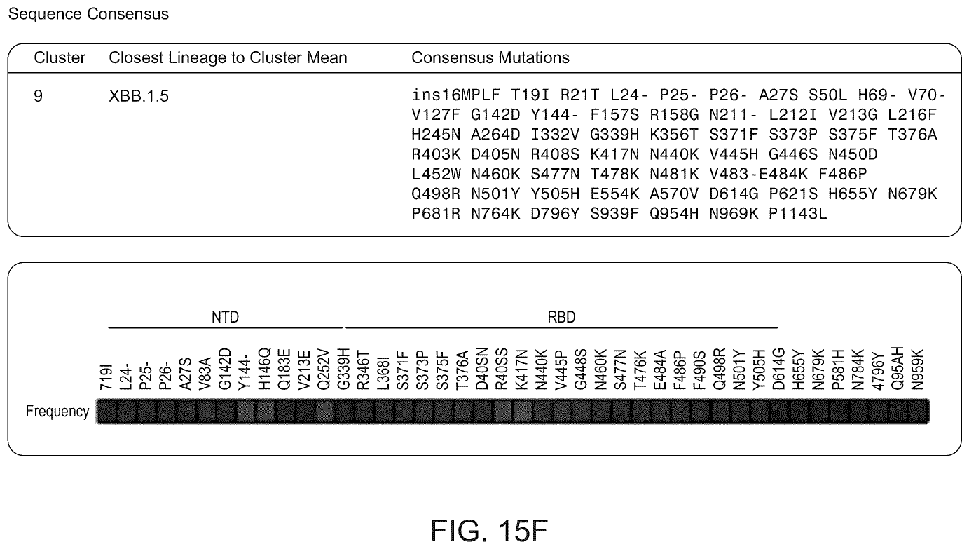

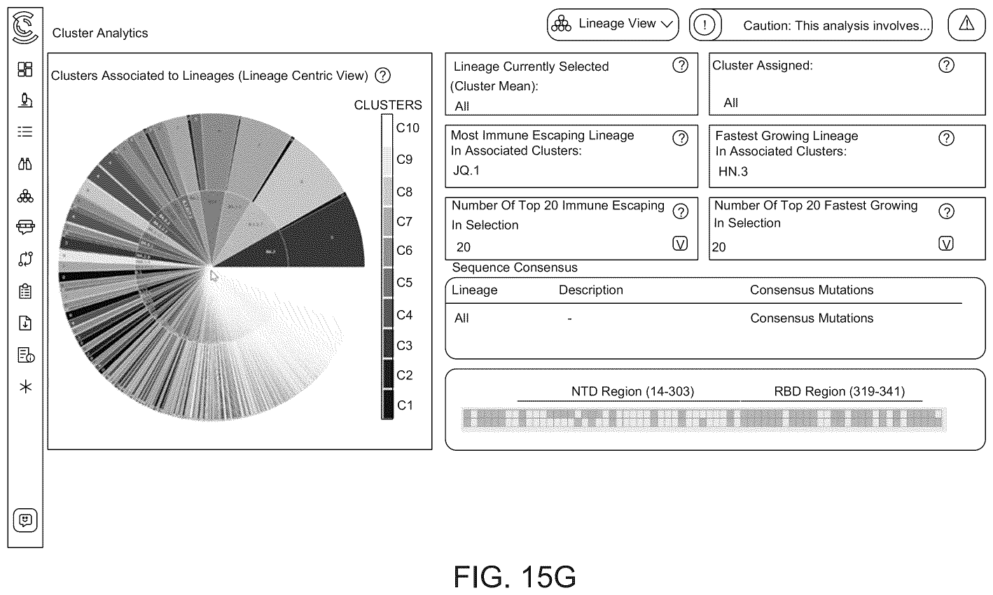

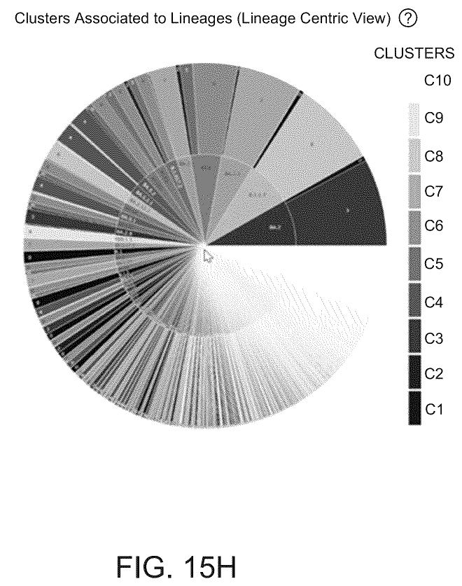

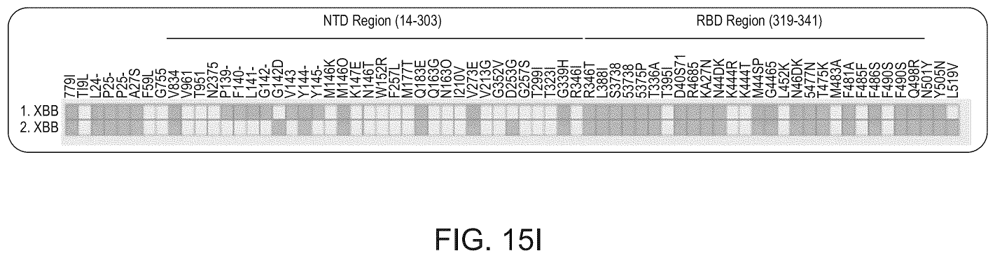

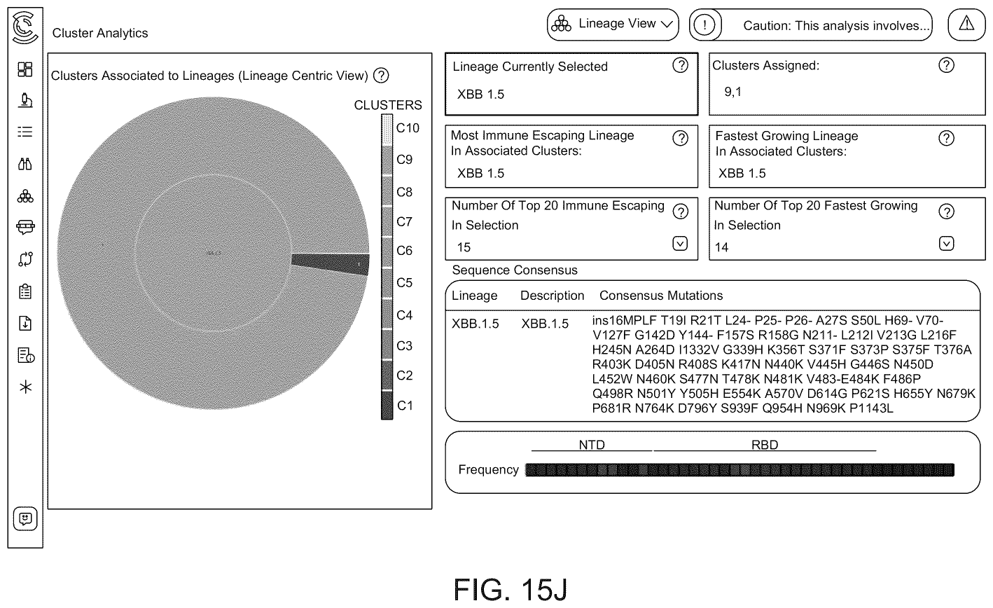

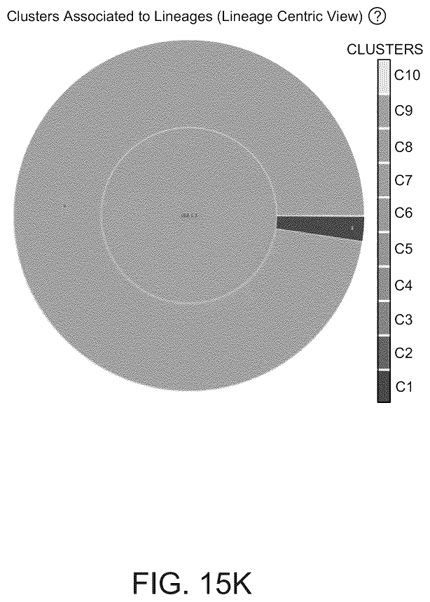

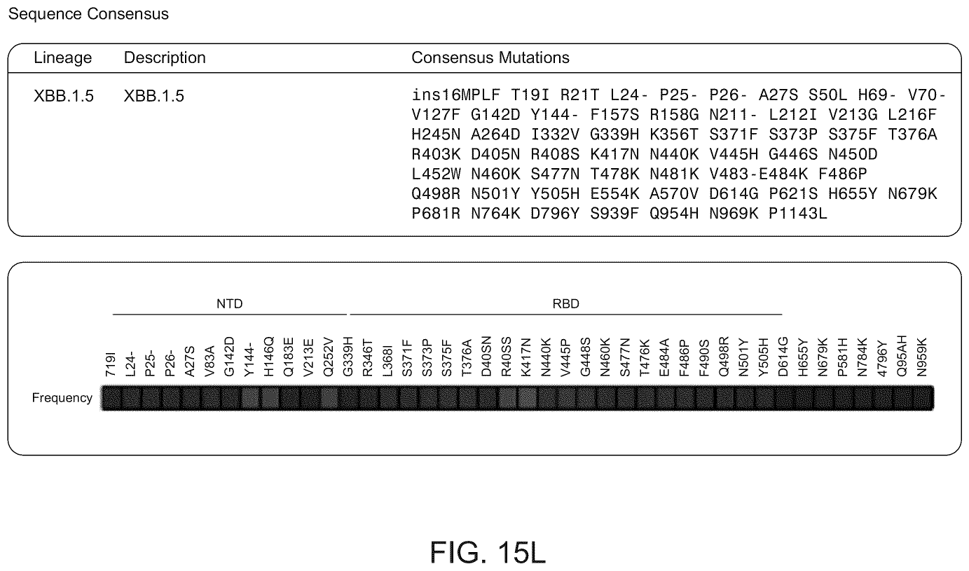

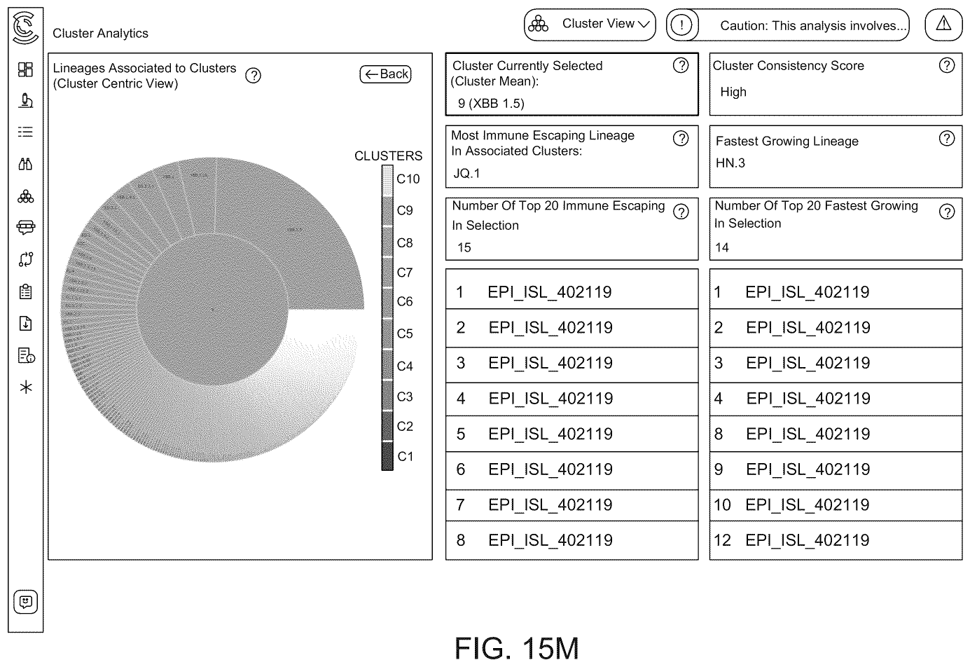

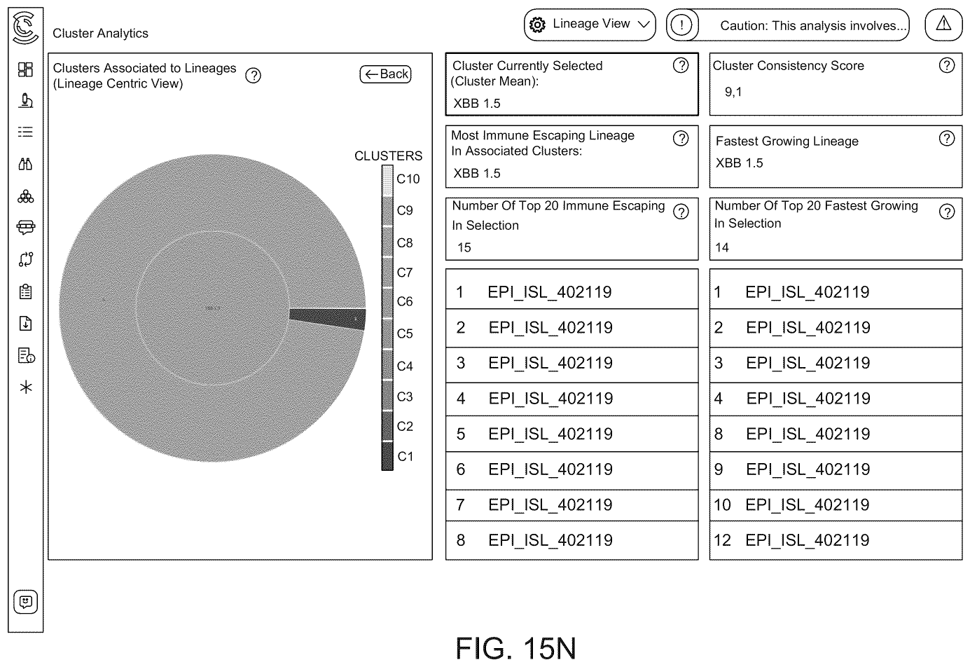

Turning to FIGS. 15A-15M, in certain embodiments, technologies of the present disclosure include approaches for rendering and/or displaying, e.g., via a GUI, representations of clusters and their association with various lineages of a particular pathogen, such as a virus. For example, FIGS. 15A-15M show graphical tools allowing a user to view correspondences between clusters and various lineages of a SARS-CoV-2 virus. In the visual displays shown in FIGS. 15A-15M, clusters are color-coded, and lineages identified via alphanumeric text tags.

### E. SOFTWARE, COMPUTER SYSTEM, AND NETWORK ENVIRONMENT

Certain embodiments described herein make use of computer algorithms in the form of software instructions executed by a computer processor. In certain embodiments, the software instructions include a machine learning module, also referred to herein as artificial intelligence software. As used herein, a machine learning module refers to a computer implemented process (e.g., a software function) that implements one or more specific machine learning algorithms, such as an artificial neural network (ANN), random forest, decision trees, support vector machines, and the like, in order to determine, for a given input, one or more output values. In certain embodiments, the input comprises alphanumeric data which can include numbers, words, phrases, or lengthier strings, for example. In certain embodiments, the one or more output values comprise values representing numeric values, words, phrases, or other alphanumeric strings. In certain embodiments, the one or more output values comprise an identification of one or more response strings (e.g., selected from a database).

In certain embodiments, machine learning modules implementing machine learning techniques are trained, for example using datasets that include categories of data described herein. Such training may be used to determine various parameters of machine learning algorithms implemented by a machine learning module, such as weights associated with layers in neural networks. In certain embodiments, once a machine learning module is trained, e.g., to accomplish a specific task such as identifying certain response strings, values of determined parameters are fixed and the (e.g., unchanging, static) machine learning module is used to process new data (e.g., different from the training data; e.g., infer a result) and accomplish its trained task without further updates to its parameters (e.g., the machine learning module does not receive feedback and/or updates). In certain embodiments, machine learning modules may receive feedback, e.g., based on automated review of accuracy or human user review of accuracy, and such feedback may be used as additional training data, to dynamically update the machine learning module. In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions of an ANN module may be carried out via specialized hardware (e.g., via an application specific integrated circuit (ASIC), field programmable gate arrays (FPGAs)).

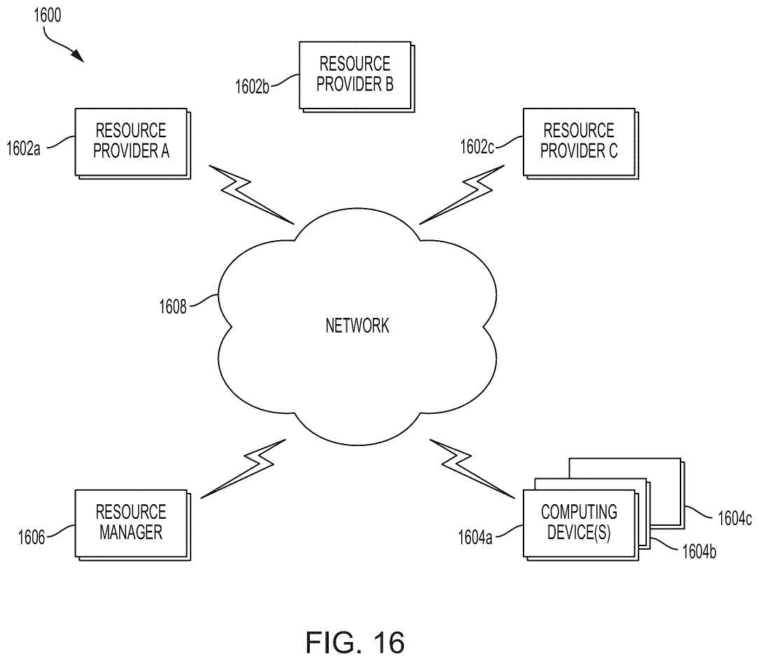

As shown in FIG. 16, an implementation of a network environment 1600 for use in providing systems, methods, and architectures as described herein is shown and described. In brief overview, referring now to FIG. 16, a block diagram of an exemplary cloud computing environment 1600 is shown and described. The cloud computing environment 1600 may include one or more resource providers 1602a, 1602b, 1602c (collectively, 1602). Each resource provider 1602 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 1602 may be connected to any other resource provider 1602 in the cloud computing environment 1600. In some implementations, the resource providers 1602 may be connected over a computer network 1608. Each resource provider 1602 may be connected to one or more computing device 1604a, 1604b, 1604c (collectively, 1604), over the computer network 1608.

The cloud computing environment 1600 may include a resource manager 1606. The resource manager 1606 may be connected to the resource providers 1602 and the computing devices 1604 over the computer network 1608. In some implementations, the resource manager 1606 may facilitate the provision of computing resources by one or more resource providers 1602 to one or more computing devices 1604. The resource manager 1606 may receive a request for a computing resource from a particular computing device 1604. The resource manager 1606 may identify one or more resource providers 1602 capable of providing the computing resource requested by the computing device 1604. The resource manager 1606 may select a resource provider 1602 to provide the computing resource. The resource manager 1606 may facilitate a connection between the resource provider 1602 and a particular computing device 1604. In some implementations, the resource manager 1606 may establish a connection between a particular resource provider 1602 and a particular computing device 1604. In some implementations, the resource manager 1606 may redirect a particular computing device 1604 to a particular resource provider 1602 with the requested computing resource.

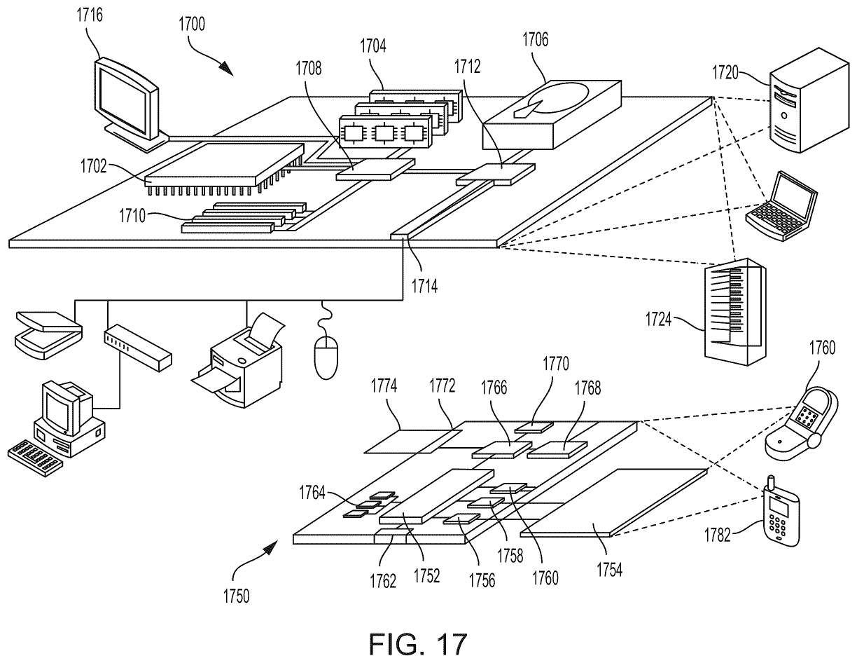

FIG. 17 shows an example of a computing device 1700 and a mobile computing device 1750 that can be used to implement the techniques described in this disclosure. The computing device 1700 is intended to represent various forms of digital computers, such as laptops, desktops, workstations, personal digital assistants, servers, blade servers, mainframes, and other appropriate computers. The mobile computing device 1750 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 1700 includes a processor 1702, a memory 1704, a storage device 1706, a high-speed interface 1708 connecting to the memory 1704 and multiple high-speed expansion ports 1710, and a low-speed interface 1712 connecting to a low-speed expansion port 1714 and the storage device 1706. Each of the processor 1702, the memory 1704, the storage device 1706, the high-speed interface 1708, the high-speed expansion ports 1710, and the low-speed interface 1712, are interconnected using various busses, and may be mounted on a common motherboard or in other manners as appropriate. The processor 1702 can process instructions for execution within the computing device 1700, including instructions stored in the memory 1704 or on the storage device 1706 to display graphical information for a GUI on an external input/output device, such as a display 1716 coupled to the high-speed interface 1708. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 1704 stores information within the computing device 1700. In some implementations, the memory 1704 is a volatile memory unit or units. In some implementations, the memory 1704 is a non-volatile memory unit or units. The memory 1704 may also be another form of computer-readable medium, such as a magnetic or optical disk.

The storage device 1706 is capable of providing mass storage for the computing device 1700. In some implementations, the storage device 1706 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 1702), perform one or more methods, such as those described above. The instructions can also be stored by one or more storage devices such as computer- or machine-readable mediums (for example, the memory 1704, the storage device 1706, or memory on the processor 1702).

The high-speed interface 1708 manages bandwidth-intensive operations for the computing device 1700, while the low-speed interface 1712 manages lower bandwidth-intensive operations. Such allocation of functions is an example only. In some implementations, the high-speed interface 1708 is coupled to the memory 1704, the display 1716 (e.g., through a graphics processor or accelerator), and to the high-speed expansion ports 1710, which may accept various expansion cards (not shown). In the implementation, the low-speed interface 1712 is coupled to the storage device 1706 and the low-speed expansion port 1714. The low-speed expansion port 1714, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 1700 may be implemented in a number of different forms, as shown in the figure. For example, it may be implemented as a standard server 1720, or multiple times in a group of such servers. In addition, it may be implemented in a personal computer such as a laptop computer 1722. It may also be implemented as part of a rack server system 1724. Alternatively, components from the computing device 1700 may be combined with other components in a mobile device (not shown), such as a mobile computing device 1750. Each of such devices may contain one or more of the computing device 1700 and the mobile computing device 1750, and an entire system may be made up of multiple computing devices communicating with each other.

The mobile computing device 1750 includes a processor 1752, a memory 1764, an input/output device such as a display 1754, a communication interface 1766, and a transceiver 1768, among other components. The mobile computing device 1750 may also be provided with a storage device, such as a micro-drive or other device, to provide additional storage. Each of the processor 1752, the memory 1764, the display 1754, the communication interface 1766, and the transceiver 1768, are interconnected using various buses, and several of the components may be mounted on a common motherboard or in other manners as appropriate.

The processor 1752 can execute instructions within the mobile computing device 1750, including instructions stored in the memory 1764. The processor 1752 may be implemented as a chipset of chips that include separate and multiple analog and digital processors. The processor 1752 may provide, for example, for coordination of the other components of the mobile computing device 1750, such as control of user interfaces, applications run by the mobile computing device 1750, and wireless communication by the mobile computing device 1750.

The processor 1752 may communicate with a user through a control interface 1958 and a display interface 1756 coupled to the display 1754. The display 1754 may be, for example, a TFT (Thin-Film-Transistor Liquid Crystal Display) display or an OLED (Organic Light Emitting Diode) display, or other appropriate display technology. The display interface 1756 may comprise appropriate circuitry for driving the display 1754 to present graphical and other information to a user. The control interface 1758 may receive commands from a user and convert them for submission to the processor 1752. In addition, an external interface 1762 may provide communication with the processor 1752, so as to enable near area communication of the mobile computing device 1750 with other devices. The external interface 1762 may provide, for example, for wired communication in some implementations, or for wireless communication in other implementations, and multiple interfaces may also be used.

The memory 1764 stores information within the mobile computing device 1750. The memory 1764 can be implemented as one or more of a computer-readable medium or media, a volatile memory unit or units, or a non-volatile memory unit or units. An expansion memory 1774 may also be provided and connected to the mobile computing device 1750 through an expansion interface 1772, which may include, for example, a SIMM (Single In Line Memory Module) card interface. The expansion memory 1774 may provide extra storage space for the mobile computing device 1750, or may also store applications or other information for the mobile computing device 1750. Specifically, the expansion memory 1774 may include instructions to carry out or supplement the processes described above, and may include secure information also. Thus, for example, the expansion memory 1774 may be provide as a security module for the mobile computing device 1750, and may be programmed with instructions that permit secure use of the mobile computing device 1750. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 1752), perform one or more methods, such as those described above. The instructions can also be stored by one or more storage devices, such as one or more computer- or machine-readable mediums (for example, the memory 1764, the expansion memory 1774, or memory on the processor 1752). In some implementations, the instructions can be received in a propagated signal, for example, over the transceiver 1768 or the external interface 1762.

The mobile computing device 1750 may communicate wirelessly through the communication interface 1766, which may include digital signal processing circuitry where necessary. The communication interface 1766 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1768 using a radio-frequency. In addition, short-range communication may occur, such as using a Bluetooth®, Wi-Fi™, or other such transceiver (not shown). In addition, a GPS (Global Positioning System) receiver module 1770 may provide additional navigation- and location-related wireless data to the mobile computing device 1750, which may be used as appropriate by applications running on the mobile computing device 1750.

The mobile computing device 1750 may also communicate audibly using an audio codec 1760, which may receive spoken information from a user and convert it to usable digital information. The audio codec 1760 may likewise generate audible sound for a user, such as through a speaker, e.g., in a handset of the mobile computing device 1750. Such sound may include sound from voice telephone calls, may include recorded sound (e.g., voice messages, music files, etc.) and may also include sound generated by applications operating on the mobile computing device 1750.

The mobile computing device 1750 may be implemented in a number of different forms, as shown in the figure. For example, it may be implemented as a cellular telephone 1780. It may also be implemented as part of a smart-phone 1782, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, various modules described herein can be separated, combined or incorporated into single or combined modules. Modules depicted in the figures are not intended to limit the systems described herein to the software architectures shown therein.

### F. EXAMPLES

### i. Example 1: Forward Looking System Parameter Sweep and Prediction Generation

This example demonstrates parameter optimization of an example forward looking system for predicting immune escaping mutations and its use in generating predictions of mutation candidates for various vaccination/breakthrough conditions.

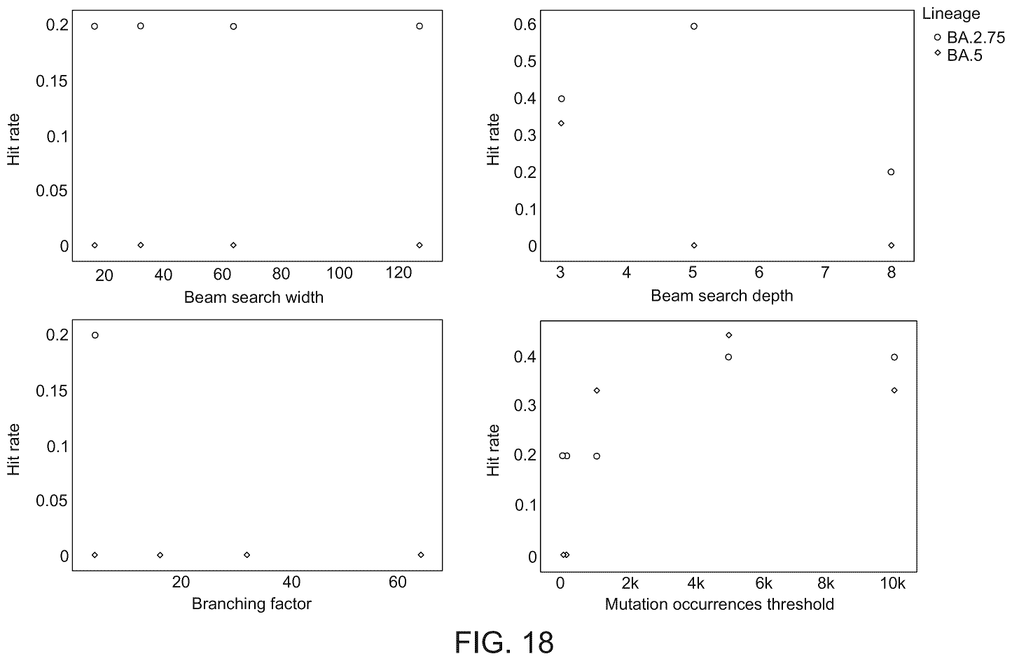

In this example, a hit rate for a lineage was defined as the proportion of hallmark mutations of its sub-lineages that were found in the top 5 recommended mutations of a forward-looking system run. Forward looking system (FLS) runs were performed for 4 different infection/breakthrough conditions (3 doses, 3 doses+BA.1/BA.2/BA.4) and the obtained results were averaged. The values of the hit rate metric were used to estimate ideal values for certain hyperparameters—namely, beam search depth, beam search width, branching factor, and mutation occurrences threshold—for the FLS process. Results of the runs for a BA.2.75 and BA.5 lineages are shown in FIG. 18. From this approach ideal hyper parameters as follows were determined.
Depth=3;
Branching factor=4;
Threshold=5000; and
Beam width=64.

### FLS Runs

Results for individual runs of the FLS for BA.2.75, BA.4, and BA.5 are provided below. Beginning with each lineage as an initial, starting, sequence, the FLS was used to predict potential immune escaping mutations based on four different host immunity experiences, namely, vaccination via three doses (of wild-type strain) alone, and triple-vaccination plus breakthrough infections from BA.1, BA.2, and BA.4/5 variants. As described herein, host immunity conditions/experiences dictate epitope weights, thereby influencing the neutralization score given a sequence.

As shown below, the FLS suggested mutations at multiple positions, several of which (identified via asterisks (*)) are positions that have already been found to be mutated in the set of hallmark mutations for various BA.2.75, BA.4, and BA.4 sublineages.

TABLE 1
FLS-suggested positions as determined for lineages
originating from BA.2.75. Asterisks denote positions
that have already been found to be mutated in hallmarks
mutations for existing BA.2.75 sublineages.

| Vaccination/breakthrough condition | FLS-suggested positions |
|---|---|
| Three doses | T323, F490, R346*, P251, F486 |
| Three doses + BA.1 breakthrough | Y145, F486*, T323, R346*, L452* |
| Three doses + BA.2 breakthrough | F490, N450, F486*, Y145, A520 |
| Three doses + BA.4/5 breakthrough | T323, P251, N450, F486*, F490 |

The BA.2.75 hallmark NTD/RBD mutations are as follows: T19I, L24-, P25-, P26-, A27S, G142D, K147E, W152R, F157L, I210V, V213G, G257S, G339H, S371F, S373P, S375F, T376A, D405N, R408S, K417N, N440K, G446S, N460K, S477N, T478K, E484A, Q498R, N501Y, Y505H.

BA.2.75 sublineages include additional hallmark RBD/NTD mutations, as listed below, with asterisks identifying positions in the set of hallmark mutations that were identified via the FLS run, as shown in Table 1, above:
BA.2.75.2: R346T*, F486S*
BA.2.75.4: L452R*
BA.2.75.5: K356T
BA.2.75.6: R346T*
BA.2.75.7: F486S*
BA.2.75.8: L452Q*
BA.2.75.9: R346T*
BL.1: R346T*

Table 2, below, lists sites of immune escaping mutations as predicted via the FLS starting with BA.4.

TABLE 2
FLS-suggested positions as determined for BA.4. Asterisks denote
positions mutated in hallmarks mutations for BA.4 sublineages.

| Vaccination/breakthrough condition | FLS-suggested positions |
|---|---|
| Three doses | K147, Y145, R346*, H146, T323 |
| Three doses + BA.1 breakthrough | R346*, H146, Y145, K147, |
| | W152* |
| Three doses + BA.2 breakthrough | F490, N450, Y145, H146, R346* |
| Three doses + BA.4/5 breakthrough | P251, R346*, F490, N450, T323 |

BA.4 hallmark NTD/RBD mutations are as follows: T19I, L24-, P25-, P26-, A27S, H69-, V70-, G142D, V213G, G339D, S371F, S373P, S375F, T376A, D405N, R408S, K417N, N440K, L452R, S477N, T478K, E484A, F486V, Q498R, N501Y, Y505H.

BA.4 sublineages include additional hallmark RBD/NTD mutations, as listed below, with asterisks identifying positions in the set of hallmark mutations that were identified via the FLS run, as shown in Table 2, above:
BA.4.1.7: Q52R
BA.4.1.8: R346T*
BA.4.1.9: R346T*
BA.4.5: G181E
BA.4.6: R346T*
BA.4.6.1: W152L*, R346T*
BA.4.7: R346S*

Table 3, below, lists sites of immune escaping mutations as predicted via the FLS starting with BA.5.

TABLE 3
FLS-suggested positions as determined for BA.5. Asterisks denote
positions mutated in hallmarks mutations for BA.5 sublineages.

| Vaccination/breakthrough condition | FLS-suggested positions |
|---|---|
| Three doses | K147, T250, N450*, P251, Y145 |
| Three doses + BA.1 breakthrough | H146, K147, R346*, F490, Y145 |
| Three doses + BA.2 breakthrough | F490, N450*, Y145, H146, R346* |
| Three doses + BA.4/5 breakthrough | P251, R346*, F490, N450*, T323 |

BA.5 hallmark NTD/RBD mutations are as follows: T19I, L24-, P25-, P26-, A27S, H69-, V70-, G142D, V213G, G339D, S371F, S373P, S375F, T376A, D405N, R408S, K417N, N440K, L452R, S477N, T478K, E484A, F486V, Q498R, N501Y, Y505H.

BA.5 sublineages include additional hallmark RBD/NTD mutations, as listed below, with asterisks identifying positions in the set of hallmark mutations that were identified via the FLS run, as shown in Table 3, above:
BA.5.1.3: V289I
BA.5.1.12: V445A
BA.5.1.18: R346T*
BA.5.1.20: R346T*
BA.5.1.26: R346T*
BA.5.1.27: R346T*
BA.5.1.28: R346T*
BA.5.2.6: R346T*
BA.5.2.7: K444M
BA.5.2.32: N450D*
BA.5.5.1: T76I, N450D*
BA.5.6.2: K444T
BA.5.9: R346I*
BA.5.10: A262S
BA.5.10.1: A262S, R346T*
BE.1.2: R346T*
BF.4: T259A
BF.6: G181A
BF.7: R346T*
BF.11: R346T*
BF.12: F486I
BF.13: R346S*
BF.14: N450D*
BF.15: Y248H

### FLS Runs: Prediction

The FLS system was also used to generate predictions of potential immune escaping mutations for XBB, BQ.1, and BM.1-based lineages, based on the four host immunity conditions described above.

Results of the FLS predictions are shown in Tables 4-6, below, with hallmark mutations for each beginning variant sequence included below each table.

TABLE 4
FLS-suggested positions as determined for XBB.

| Vaccination/breakthrough condition | FLS-suggested positions |
|---|---|
| Three doses | T323, S494, N450, T250, P251 |
| Three doses + BA.1 breakthrough | N450, W152, K147, P251, T323 |
| Three doses + BA.2 breakthrough | T323, S494, L452, P251, A520 |
| Three doses + BA.4/5 breakthrough | T323, S494, A520, L452, P251 |

XBB hallmark NTD/RBD mutations: T19I, L24-, P25-, P26-, A27S, V83A, G142D, Y144-, H146O, Q183E, V213E, G339H, R346T, L368I, S371F, S373P, S375F, T376A, D405N, R408S, K417N, N440K, V445P, G446S, N460K, S477N, T478K, E484A, F486S, F490S, 0498R, N5T1Y, Y505H.

TABLE 5
FLS-suggested positions as determined for BQ.1.

| Vaccination/breakthrough condition | FLS-suggested positions |
|---|---|
| Three doses | K147, T323, D253, P251, R346 |
| Three doses + BA.1 breakthrough | Y145, K147, H146, R346, W152 |
| Three doses + BA.2 breakthrough | K147, R346, F490, W152, Y145 |
| Three doses + BA.4/5 breakthrough | P251, R346, F490, T323, W152 |

BQ.1 hallmark NTD/RBD mutations: T19I, L24-, P25-, P26-, A27S, H69-, V70-, G1421D, V213G, G3391D, S371F, S373P, S375F, T376A, D405N, R408S, K417N, N440K, K444T, L452R, N460K, S477N, T478K, E484A, F486V, Q498R, N501Y, Y505H.

TABLE 5
FLS-suggested positions as determined for BM.1.

| Vaccination/breakthrough condition | FLS-suggested positions |
|---|---|
| Three doses | H146, R346, P251, N450, T323 |
| Three doses + BA.1 breakthrough | L452, R346, N450, F490, K444 |
| Three doses + BA.2 breakthrough | R346, S494, A520, P251, T323 |
| Three doses + BA.4/5 breakthrough | R346, S494, P251, T323, A520 |

BM.1 hallmark NTD/RBD mutations: T19I, L24-, P25-, P26-, A27S, K147E, W152R, F157L, I210V, V213G, G257S, G339H, S371F, S373P, S375F, T376A, D405N, R408S, K417N, N440K, G446S, N460K, S477N, T478K, E484A, F486S, Q498R, N501Y, Y505H.

Accordingly, this example shows that FLS technologies of the present disclosure can successfully predict mutations likely to contribute to immune escape potential in the context of SARS-CoV-2.

### EQUIVALENTS

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

While the invention has been particularly shown and described with reference to specific preferred embodiments, it should be understood by those skilled in the art that various changes in form and detail may be made therein without departing from the spirit and scope of the invention as defined by the appended claims.

1. A method for predicting one or more mutations of a viral variant likely to increase immune evasion, the method comprising:
(a) receiving and/or accessing, by a processor of a computing device, sequence data for a selected variant polypeptide, wherein the selected variant polypeptide is a selected variant of a particular reference polypeptide;
(b) generating, by the processor, a plurality of candidate mutations;
(c) generating, by the processor, a plurality of candidate mutation combinations, each candidate mutation combination comprising a particular combination of one or more of the plurality of candidate mutations and determining, for each candidate mutation combination, a corresponding value of a neutralization score, thereby determining, values of the neutralization score for each of the plurality of candidate mutation combinations;
(d) selecting, by the processor, based on the values of the neutralization score determined for the plurality of candidate mutation combinations, at least one of the particular candidate mutation combinations as a set of predicted mutations that are likely to increase immune evasion; and
(e) storing and/or providing, by the processor, the set of predicted mutations for display and/or further processing.

2. The method of claim 1, wherein the sequence data for the selected variant polypeptide comprises sequences of one or more particular subunits of the viral polypeptide.

3. The method of claim 2, wherein the sequence data for the selected variant polypeptide comprises one or both of (i) and (ii) as follows: (i) a sequence of a receptor binding domain (RBD) of a SARS-CoV-2 spike protein variant; and (ii) a sequence of a N-terminal domain (NTD) of a SARS-CoV-2 spike protein variant.

4. The method of claim 1, comprising:
receiving and/or accessing, by the processor, a plurality of submitted variant sequences, each submitted variant sequence representing at least a portion of a circulating variant of the reference polypeptide;
determining, by the processor, for each of the plurality of submitted variant sequences, values one or more immune escape scores;
selecting, by the processor, a subset of the submitted variant sequences based on the values of the one or more immune escape scores determined for the submitted variant sequences; and
performing, by the processor, for each particular submitted variant sequence of the selected subset, steps (a)-(e) using the particular submitted variant sequence as the selected variant sequence, thereby determining, for each sequence of the selected subset of submitted variant sequences, a corresponding set of predicted mutations.

5. The method of claim 4, wherein the one or more immune escape scores comprise an epitope alteration score and/or a semantic change score.

6. The method of claim 1, wherein step (b) comprises one or both of (i) and (ii) as follows:
(i) selecting, as the plurality of candidate mutations, a subset of potential mutations having been identified in previously sequenced variant polypeptides; and
(ii) selecting the plurality of candidate mutations based on determined infectivity/fitness score values by:
generating a plurality of potential mutations;
determining, for each of the plurality of potential mutations, values of a corresponding infectivity/fitness score; and
selecting a subset of the plurality of potential mutations for use as the plurality of candidate mutations based on the determined infectivity/fitness score values.

7. (canceled)

8. The method of claim 1, wherein the neutralization score is determined, for a particular candidate mutation combination, based on, for each of a plurality of selected epitopes, a number of positions mutated on the epitope relative to a particular reference polypeptide.

9. The method of claim 1, wherein the neutralization score is determined, for a particular candidate mutation combination, based on, for each of a plurality of selected epitopes, a function of a number of positions mutated on the epitope and wherein the method comprises one or both of the following:
determining and/or selecting the plurality of selected epitopes according to a particular vaccination/breakthrough condition; and
determining and/or assigning an epitope weight to each of the plurality of selected epitopes according to the particular vaccination/breakthrough condition and scaling the function of the number of mutated positions on each epitope according to the corresponding epitope weight.

10-11. (canceled)

12. The method of claim 1, comprising performing steps (a)-(d) for each of a plurality of selected variant sequences to generate a plurality of sets of predicted mutations, and causing display of the plurality of sets of predicted mutations.

13. The method of claim 1, comprising performing steps (a)-(d) for each of a plurality of variant sequences, each corresponding to a particular subunit or region of a viral variant polypeptide.

14. The method of claim 1, comprising repeatedly performing steps (a)-(d) over time, thereby regularly updating the set of predicted mutations as new sequence and/or epitope data is obtained.

15. The method of claim 1, comprising:
repeatedly receiving and/or accessing, by the processor, sequence data comprising sequences of sequenced variants;
comparing, by the processor, the sequence data to the set of predicted mutations; and
responsive to identifying presence of at least a portion of the set of predicted mutations within one or more of the sequences, causing, by the processor, transmission and/or display of an alert to one or more users.

16. A method for evaluating and tracking evolution of viral lineages via an interactive decision support system, the method comprising:
(a) receiving and/or accessing, by a processor of a computing device, viral lineage data from one or more databases, the viral lineage data comprising, for each particular lineage of a plurality of viral lineages, one or more of (i) through (iv) as follows:
(i) a lineage name identifying the particular lineage;
(ii) a lineage description;
(iii) a corresponding set of consensus mutations for the particular lineage; and
(iv) a submissions dataset comprising a plurality of submitted sequences identified as belonging to the particular lineage;
(b) performing, by the processor, one or both of (A) and (B) as follows:
(A) causing rendering of a visual representation of at least a portion of the viral lineage data; and
(B) using the viral lineage data to determine, for each of at least a portion of the plurality of viral lineages, values one or more epitope conservation metrics and causing rendering of a graphical representation of the determined values of the one or more epitope conservation metrics for graphical display and/or providing the determined values one or more epitope conservation for further processing.

17-19. (canceled)

20. A method for forecasting prevalence and/or distribution of a plurality variants of a circulating pathogen, the method comprising:
(a) receiving and/or accessing, by a processor of a computing device, sequence data for the circulating pathogen, the sequence data comprising, a plurality of variant sequences, each (i) representing a sequence of a variant of a particular polypeptide of the circulating pathogen and (ii) associated with a particular time;
(b) for each particular time point of a plurality of time points, identifying and assigning one or more of the plurality of variant sequences that are associated with the particular time point to a particular cluster of a set of clusters, thereby sub-dividing the plurality of variant sequences across the set of clusters and tracking the distribution of variant sequences across the set of clusters over time; and
(c) performing, by the processor, one or both of (A) and (B) as follows:
(A) causing rendering of a visual representation of the distribution of variant sequences across the set of clusters; and
(B) using distribution of variant sequences across the set of clusters and its variation over time to generate a projected distribution of variant sequences across the one or more clusters at a current and/or future time point.

21. The method of claim 20, wherein the variant sequences are sequences of variants of a particular viral polypeptide.

22. The method of claim 20, wherein step (b) comprises one or more of (A), (B), and (C) as follows:
(A) determining, for each of the plurality of variant sequences, using a machine learning model, a corresponding characteristic vector, thereby determining a plurality of characteristic vectors, each corresponding to a particular one of the plurality of variant sequences; and using the plurality of characteristic vectors to assign each of the variant sequences to a particular cluster of the set of clusters;
(B) determining, by the processor, for one or more reference time point(s), the set of clusters based on one or more of the plurality of variant sequences associated with the one or more reference time point(s); and at other time points, assigning, by the processor, variant sequences (associated with the other time points) to a particular cluster of the set of clusters determined at for the reference time point(s); and
(C) determining, by the processor, at each particular time point of the plurality of time points, an initial set of clusters based on one or more of the plurality of variant sequences associated with the particular time point, thereby determining a plurality of initial sets of clusters, each associated with a particular time point of the plurality of time points and matching, by the processor, corresponding clusters between the plurality of initial sets of clusters to determine, and assign variant sequences to, a common set of clusters.

23. The method of claim 22, comprising assigning each of the plurality of variant sequences to a particular cluster of the set of clusters based at least in part on its corresponding characteristic vector and/or one or more reduced dimensionality versions thereof.

24. (canceled)

25. The method of claim 22, wherein the machine learning model is a large language model (LLM), wherein the LLM is a trained model, having been previously trained to predict, based on receipt of a partially masked and/or incomplete input polypeptide sequence, types of masked and/or remaining sequence locations.

26-27. (canceled)

28. The method of claim 20, comprising:
determining, by the processor, a reference set of variant sequences comprising a plurality of variant sequences associated with each of one or more reference time point(s);
using the reference set of variant sequences to generate a training dataset comprising characteristic vectors and/or reduced dimensionality versions thereof for each of the variant sequences of the reference set; and
training a clustering model according to the training dataset, thereby determining a trained clustering model and the set of clusters.

29. The method of claim 28, wherein the trained clustering model receives, as input, a characteristic vector corresponding to a particular variant sequence, and/or reduced dimensionality version thereof, and determines, as output an assignment of a particular cluster of the set of clusters for the particular variant sequence.

30. The method of claim 28, comprising using the trained clustering model to assign variant sequences to a particular cluster of the set of clusters.

31-43. (canceled)

44. A system for predicting one or more mutations of a viral variant likely to increase immune evasion, the system comprising:
a processor of a computing device; and
memory having instructions stored thereon, wherein the instructions, when executed by the processor, cause the processor to:
(a) receive and/or access sequence data for a selected variant polypeptide, wherein the selected variant polypeptide is a selected variant of a particular reference polypeptide;
(b) generate a plurality of candidate mutations;
(c) generate a plurality of candidate mutation combinations, each candidate mutation combination comprising a particular combination of one or more of the plurality of candidate mutations and determining, for each candidate mutation combination, a corresponding value of a neutralization score, thereby determining, values of the neutralization score for each of the plurality of candidate mutation combinations;
(d) select, based on the values of the neutralization score determined for the plurality of candidate mutation combinations, at least one of the particular candidate mutation combinations as a set of predicted mutations that are likely to increase immune evasion; and
(e) store and/or provide the set of predicted mutations for display and/or further processing.

45-47. (canceled)
